(19) United States
(12) Patent Application Publication
(10) Pub. No.: US 2026/0072014 A1
(43) Pub. Date: Mar. 12, 2026

(54) PHARMACEUTICAL COMPOSITION FOR PREVENTION OR TREATMENT OF NEUROLOGICAL DISEASES COMPRISING HIGHLY POTENT STEM CELLS EXPRESSING TIMP-1, MCP-1, GROa, AND IL-6, AND METHOD FOR SELECTING HIGHLY POTENT STEM CELLS

(71) Applicant: MEDINNO INC., Seoul (KR)

(72) Inventors: Kyung Min JOO, Seoul (KR); Chung Kwon KIM, Seoul (KR); Geun-Hyoung HA, Gyeonggi-do (KR)

(21) Appl. No.: 19/106,444

(22) PCT Filed: Jul. 11, 2023
(86) PCT No.: PCT/KR2023/009891

§ 371 Date: Feb. 25, 2025

(30) Foreign Application Priority Data
Aug. 26, 2022 (KR) 10-2022-0107959

(51) Int. Cl. G01N 33/50; A61K 35/30; A61P 25/00; C12Q 1/6883

(52) U.S. Cl. CPC G01N 33/5073; A61K 35/30; A61P 25/00; C12Q 1/6883; C12Q 2600/158; G01N 2333/475; G01N 2333/5412; G01N 2333/8146

(57) ABSTRACT

The present invention relates to a method for selecting highly potent stem cells for the treatment of neurological disease using TIMP-1, MCP-1, GROα, and IL-6, and a pharmaceutical composition for the prevention or treatment of neurological disease. According to the present invention, a specific combination of markers including TIMP-1, MCP-1, GROα, and IL-6 can be used as a biomarker for selecting stem cells with superior therapeutic (ameliorating) potential in the treatment of neurological disease. Moreover, the application of stem cells exhibiting increased expression or activity of TIMP-1, MCP-1, GROα, and IL-6 factors or the use of substances that regulate (particularly upregulate) these factors in stem cells has a remarkable therapeutic effect on neurological disease due to having high efficacy (potency) and efficiency. Furthermore, the present invention presents the higher therapeutic potential of allogeneic stem cell transplantation.

### TECHNICAL FIELD

The present invention relates to a method for selecting highly potent stem cells for the prevention or treatment of neurological disease using TIMP-1, MCP-1, GROα, and IL-6, and a pharmaceutical composition for the prevention or treatment of neurological disease comprising the highly potent stem cells.

The present invention claims priority based on Korean Patent Application No. 10-2022-0107959, filed on Aug. 26, 2022, and all contents disclosed in the specifications and drawings of these applications are incorporated herein by reference.

### BACKGROUND ART

Neurological disease is known to occur due to the loss of neurons in the central nervous system or peripheral nervous system and is currently recognized as diseases for which no specific treatment has been developed. A representative example of neurological disease, stroke, is defined as a disruption in blood flow to brain tissue. This condition occurs when a specific region of the brain becomes impaired or when the heart's pumping function is completely compromised. Specifically, a stroke occurs when blood flow to a particular region of the brain is obstructed or disrupted due to the blockage or rupture of blood vessels leading to the brain. Specifically, this occurs when blood vessels are blocked due to the formation of an embolism or thrombus in the arteries supplying blood to the brain, a condition known as ischemic stroke. Stroke is the third leading cause of death in developed countries. In the United States, the treatment costs and productivity losses resulting from stroke-related deaths are estimated to amount to approximately $40 trillion. When ischemic stroke occurs, necrosis of brain tissue and cerebral infarction take place. Within 3 to 6 hours of onset, treatment methods such as thrombolysis, including drug administration and rehabilitation therapy, are used to promote the preservation and recovery of brain function. However, thrombolysis carries the risk of increasing cerebral hemorrhage, and since it must be performed within a few hours after cerebral infarction occurs, many patients are unable to undergo the procedure. These stroke treatment methods are limited to emergency care or merely preventing the progression of the disease, with no fundamental therapeutic measures available for regenerating already damaged brain tissue.

To overcome these limitations of modern medicine, recent efforts have focused on utilizing stem cells with regenerative effects on the nervous system for treatment. Stem cells are undifferentiated cells that can differentiate into various types of body tissues. Under appropriate conditions, they have the ability to differentiate into different tissue cells, making them applicable for treatments such as regenerating damaged tissues. Such stem cell therapies are expected to enable the regeneration of damaged neurons. In addition to direct regeneration, these stem cells are known to secrete various substances that improve the damaged environment and contribute to the regenerative process. Despite the rapid advancement of stem cell-related technologies and high expectations for their therapeutic effects on neurological disease, clinical results using mesenchymal stem cells for stroke and other degenerative neurological disease have reported limited efficacy in practice. Furthermore, while stem cell-based therapies are being attempted across various disease areas, they have not shown significant efficacy in practice. As a result, extensive research is being conducted to identify highly potent stem cells. Additionally, clinical studies using fetal-derived neural stem cells for stroke and spinal cord injury patients are underway. However, serious technical issues, such as tumor formation due to incomplete differentiation, as well as ethical concerns, have arisen.

### DISCLOSURE

### Technical Problem

In response, the inventors conducted research to find a more highly effective and efficient treatment strategy for neurological disease using stem cells. During this process, they identified for the first time the critical roles of TIMP-1, MCP-1, GROα, and IL-6 in the therapeutic activity of stem cells for neurological disease, thereby completing the present invention.

Therefore, the objective of the present invention is to provide a method for selecting highly potent stem cells for the treatment of neurological disease, which includes a step of measuring the expression levels of TIMP-1 (TIMP Metallopeptidase Inhibitor 1), MCP-1 (Monocyte Chemoattractant Protein-1), GROα (Growth-Regulated Oncogene alpha), and IL-6 (Interleukin 6) proteins or their genes.

Another objective of the present invention is to provide a method for determining the activity of stem cells for treating neurological disease ex vivo. The method includes a step of measuring and comparing the expression levels of TIMP-1, MCP-1, GROα, and IL-6 proteins or genes in stem cells.

Another objective of the present invention is to provide a marker composition for selecting highly potent stem cells for the treatment of neurological disease, which includes TIMP-1, MCP-1, GROα, and IL-6 proteins or their genes.

Another objective of the present invention is to provide a composition for selecting highly potent stem cells for the treatment of neurological disease, which includes a substance for measuring the expression levels of TIMP-1, MCP-1, GROα, and IL-6 proteins or their genes. Additionally, the present invention provides a kit for selecting highly potent stem cells for the treatment of neurological disease, which includes the above composition.

Another objective of the present invention is to provide a method for screening for a preventive or therapeutic agent for neurological disease, comprising:
(a) contacting isolated stem cells with a test substance;
(b) measuring the expression levels of TIMP-1, MCP-1, GROα, and IL-6 proteins or their genes in stem cells contacted with the test substance and in a control group of stem cells not contacted with the test substance; and
(c) selecting a test substance that increases the expression or activity levels of TIMP-1, MCP-1, GROα, and IL-6 proteins or their genes as compared to the control group cells.

Another objective of the present invention is to provide a pharmaceutical composition for the prevention or treatment of neurological disease, comprising stem cells overexpressing TIMP-1, MCP-1, GROα, and IL-6 genes or their proteins; or a culture thereof as an active ingredient.

Another objective of the present invention is to provide a composition for promoting the activity of stem cells for the prevention or treatment of neurological disease, comprising a substance that increases the expression or activity of the TIMP-1, MCP-1, GROα, and IL-6 genes or their proteins as an active ingredient.

Another objective of the present invention is to provide a method for promoting the activity of stem cells for the prevention or treatment of neurological disease, comprising a step of treating isolated stem cells with an agent that increases the expression or activity of the genes or proteins of TIMP-1, MCP-1, GROα, and IL-6.

Another objective of the present invention is to provide a pharmaceutical composition for the prevention or treatment of neurological disease, comprising isolated stem cells; and a substance that increases the expression or activity of TIMP-1, MCP-1, GROα, and IL-6 genes or their proteins as an active ingredient, and a pharmaceutical combination for the prevention or treatment of neurological disease comprising the above composition.

Another objective of the present invention is to provide a pharmaceutical composition for inducing angiogenesis, comprising stem cells that overexpress TIMP-1, MCP-1, GROα, and IL-6 genes or their proteins: or a culture thereof as an active ingredient.

Another objective of the present invention is to provide a pharmaceutical composition for inducing angiogenesis, comprising isolated stem cells; and a substance that increases the expression or activity of TIMP-1, MCP-1, GROα, and IL-6 genes or their proteins as an active ingredient, and a pharmaceutical combination for inducing angiogenesis, comprising the above composition.

However, the technical problems to be solved by the present invention are not limited to those mentioned above, and other unmentioned problems can be clearly understood by those skilled in the art to which the present invention pertains from the following description.

### Technical Solution

To achieve the aforementioned objective, the present invention provides a method for selecting highly potent stem cells for the treatment of neurological disease, comprising a step of measuring the expression levels of TIMP-1 (TIMP Metallopeptidase Inhibitor 1), MCP-1 (Monocyte Chemoattractant Protein-1), GROα (Growth-Regulated Oncogene alpha), and IL-6 (Interleukin 6) proteins or their genes.

In one embodiment of the present invention, TIMP-1, MCP-1, GROα, and IL-6 proteins or their genes may be highly expressed in the highly potent stem cells for the treatment of the neurological disease, but are not limited thereto.

In one embodiment of the present invention, wherein the stem cells may be selected from the group consisting of mesenchymal stem cells, adult stem cells, dedifferentiated stem cells, embryonic stem cells, induced pluripotent stem cells, neuronal stem cells, tissue-derived stem cells, and any combination of two or more thereof, but is not limited thereto.

In one embodiment of the present invention, the neurological disease may be one or more selected from the group consisting of ischemic brain disease, degenerative neurological disease, nerve damage disease, peripheral nerve disorders, and traumatic brain injury, but is not limited thereto.

In one embodiment of the present invention, the ischemic brain disease may be one or more selected from the group consisting of stroke, cerebral infarction, cerebral ischemia, cerebral embolism, cerebral thrombosis, transient ischemic attack (TIA), head trauma, brain circulatory metabolic disorders, brain coma, cerebral hemorrhage, neonatal intraventricular hemorrhage (IVH), cerebral arteriosclerosis, hypoxic brain injury, and neonatal hypoxic-ischemic brain injury, but is not limited thereto.

In one embodiment of the present invention, the degenerative neurological disease may be one or more selected from the group consisting of dementia, Alzheimer's disease, Parkinson's disease, progressive supranuclear palsy, multiple system atrophy, olivopontocerebellar atrophy (OPCA), Shy-Drager syndrome, striatonigral degeneration, Huntington's disease, amyotrophic lateral sclerosis (ALS), essential tremor, corticobasal ganglionic degeneration, diffuse Lewy body disease, Parkinson-ALS-dementia complex of Guam, and Pick's disease, but is not limited thereto.

In one embodiment of the present invention, the nerve damage disease may be one or more selected from the group consisting of epilepsy, spinal cord injury diseases, behavioral disorders, developmental disorders, intellectual disability, Down syndrome, and schizophrenia, but is not limited thereto.

In one embodiment of the present invention, the method for selecting highly potent stem cells for the treatment of neurological disease may comprise the following steps, but is not limited thereto:
(i) culturing stem cells;
(ii) measuring concentrations of TIMP-1, MCP-1, GROα, and IL-6 protein in the stem cell culture of step (i); and
(iii) determining and classifying the stem cells as highly potent stem cells for neurological disease treatment if TIMP-1, MCP-1, GROα, and IL-6 proteins are highly expressed in the culture.
   In One Embodiment of the Present Invention, Wherein Step (iii) is

Wherein step (ii) involves measuring the culture, and if the TIMP-1 protein concentration is 6,000 μg/mL or higher, the MCP-1 protein concentration is 6,000 pg/mL or higher, the GROα protein concentration is 500 μg/mL or higher, and the IL-6 protein concentration is 50 μg/mL or higher, the stem cells are determined to be highly potent stem cells, but is not limited thereto.

To achieve another object of the present invention, the present invention provides a method for determining the activity of stem cells for treating neurological disease ex vivo, wherein the method comprises a step of measuring and comparing the expression levels of TIMP-1, MCP-1, GROα, and IL-6 proteins or their genes in the stem cells.

To achieve yet another object of the present invention, the present invention provides a marker composition for selecting highly potent stem cells for the treatment of neurological disease, comprising TIMP-1, MCP-1, GROα, and IL-6 proteins or their genes.

To achieve yet another object of the present invention, the present invention provides a composition for selecting highly potent stem cells for the treatment of neurological disease, comprising an substance for measuring the expression levels of TIMP-1, MCP-1, GROα, and IL-6 proteins or their genes; and a kit for selecting highly potent stem cells for the treatment of neurological disease comprising wherein the composition.

Additionally, the present invention provides the use of a substance for measuring the expression levels of TIMP-1, MCP-1, GROα, and IL-6 proteins or their genes, or a composition comprising the same as an active ingredient, for selecting highly potent stem cells for the treatment of neurological disease.

Additionally, the present invention provides the use of a substance for measuring the expression levels of TIMP-1, MCP-1, GROα, and IL-6 proteins or their genes, or a composition comprising the same as an active ingredient, for the manufacture of a preparation for selecting highly potent stem cells for the treatment of neurological disease.

In one embodiment of the present invention, the substance for measuring the expression levels of the proteins may be an antibody specific to the proteins, but is not limited thereto.

In one embodiment of the present invention, the measurement of the expression levels of the gene may be performed by a substance for measuring the mRNA expression levels of the genes, but is not limited thereto. In this case, the substance for measuring the mRNA expression levels may be a probe or a primer set that specifically binds to the mRNA, but is not limited thereto.

In one embodiment of the present invention, the kit for selecting highly potent stem cells for treating neurological disease according to the present invention may comprise an instruction manual that describes the aforementioned method for selecting highly potent stem cells for treating neurological disease, but is not limited thereto.

To achieve another object of the present invention, the present invention provides a method for screening for a preventive or therapeutic agent for neurological disease, comprising:
(a) contacting isolated stem cells with a test substance;
(b) measuring the expression levels of TIMP-1, MCP-1, GROα, and IL-6 proteins or their genes in the stem cells contacted with the test substance and in a control group of stem cells not contacted with the test substance; and
(c) selecting a test substance that increases the expression or activity levels of TIMP-1, MCP-1, GROα, and IL-6 proteins or their genes as compared to the control group cells.

In one embodiment of the present invention, the stem cells may be selected from the group consisting of mesenchymal stem cells, adult stem cells, dedifferentiated stem cells, embryonic stem cells, induced pluripotent stem cells, neural stem cells, tissue-derived stem cells, and any combination of two or more thereof, but are not limited thereto.

In one embodiment of the present invention, the measurement of protein expression or activity levels may be performed using any one of the methods selected from the group consisting of Western blot, ELISA (enzyme-linked immunoassay), radioimmunoassay, radial immunodiffusion, Ouchterlony immunodiffusion, rocket immunoelectrophoresis, immunohistochemical staining, immunoprecipitation, complement fixation assay, flow cytometry, and protein chips, but is not limited thereto.

In one embodiment of the present invention, the measurement of gene expression or activity levels may be performed using a method for measuring mRNA expression or activity levels, but is not limited thereto. In this case, the measurement of mRNA expression or activity levels may be performed using any one of the following methods: reverse transcription polymerase chain reaction (RT-PCR), competitive RT-PCR, real-time RT-PCR, quantitative or semi-quantitative RT-PCR, quantitative or semi-quantitative real-time RT-PCR, in situ hybridization, RNase protection assay (RPA), northern blotting, southern blotting, RNA sequencing, DNA chip, or RNA chip, but is not limited thereto.

To achieve another object of the present invention, the present invention provides a pharmaceutical composition for the prevention or treatment of neurological disease, comprising stem cells overexpressing TIMP-1, MCP-1, GROα, and IL-6 genes or their proteins: or a culture thereof as an active ingredient.

Additionally, the present invention provides a method for the prevention, improvement, and/or treatment of neurological disease, comprising a step of administering to a subject in need thereof stem cells overexpressing TIMP-1, MCP-1, GROα, and IL-6 genes or their proteins: or a culture thereof.

Additionally, the present invention provides a method for the prevention, improvement, and/or treatment of neurological disease, comprising a step of administering to a subject in need thereof a composition containing stem cells overexpressing TIMP-1, MCP-1, GROα, and IL-6 genes or their proteins: or a culture thereof as the active ingredient.

Additionally, the present invention provides the use of stem cells overexpressing TIMP-1, MCP-1, GROα, and IL-6 genes or their proteins; or a culture thereof; or a composition containing these as the active ingredient for the prevention, improvement, and/or treatment of neurological disease.

Additionally, the present invention provides the use of stem cells overexpressing TIMP-1, MCP-1, GROα, and IL-6 genes or their proteins; or a culture thereof; or a composition containing these as the active ingredient, for the manufacture (production) of a preparation (formulation) for the prevention, improvement, and/or treatment of neurological disease.

In one embodiment of the present invention, the stem cells may be selected from the group consisting of mesenchymal stem cells, adult stem cells, induced pluripotent stem cells, embryonic stem cells, neural stem cells, tissue-derived stem cells, and any combinations of two or more thereof, but are not limited thereto.

In one embodiment of the present invention, the culture (culture medium) may be a conditioned medium comprising TIMP-1, MCP-1, GROα, and IL-6 proteins secreted by the stem cells, but is not limited thereto.

To achieve another object of the present invention, the present invention provides a composition for promoting the activity of stem cells for the prevention or treatment of neurological disease, comprising a substance that increases the expression or activity of the TIMP-1, MCP-1, GROα, and IL-6 genes or their proteins as an active ingredient.

To achieve another object of the present invention, the present invention provides a method for promoting the activity of stem cells for the prevention or treatment of neurological disease, comprising a step of treating isolated stem cells with a substance (agent) that increases the expression or activity of TIMP-1, MCP-1, GROα, and IL-6 genes or their proteins.

Additionally, the present invention provides the use of an agent that increases the expression or activity of TIMP-1, MCP-1, GROα, and IL-6 genes or their proteins, or a composition comprising the same as an active ingredient, for promoting the activity of stem cells for the prevention or treatment of neurological disease.

Additionally, the present invention provides the use of a substance that increases the expression or activity of TIMP-1, MCP-1, GROα, and IL-6 genes or their proteins, or a composition comprising the same as an active ingredient, for the manufacture of preparation for promoting the activity of stem cells for the prevention or treatment of neurological disease.

In one embodiment of the present invention, the substance that increases the expression or activity of the proteins may be one or more expression vectors comprising a polynucleotide encoding TIMP-1 protein: a polynucleotide encoding MCP-1 protein: a polynucleotide encoding GROα protein; and a polynucleotide encoding IL-6 protein, but is not limited thereto.

In one embodiment of the present invention, the activity of the stem cells may be one or more selected from the group consisting of self-renewal, neuroprotective ability (neuroprotection), inhibition of neurocyte apoptosis, vascular endothelial cell migration, angiogenesis, anti-inflammatory activity, and inhibition of reactive gliosis, but is not limited thereto.

To achieve another object of the present invention, the present invention provides a pharmaceutical composition for the prevention or treatment of neurological disease, comprising isolated stem cells; and a substance that increases the expression or activity of TIMP-1, MCP-1, GROα, and IL-6 genes or their proteins as an active ingredient, and a pharmaceutical combination for the prevention or treatment of neurological disease comprising the composition.

Additionally, the present invention provides a method for the prevention, improvement, and/or treatment of neurological disease, comprising the step of administering (in particular, co-administration or combination administration) to a subject in need thereof, isolated stem cells; and a substance for increasing the expression or activity of TIMP-1, MCP-1, GROα, and IL-6 genes or their proteins.

Additionally, the present invention provides a method for the prevention, improvement, and/or treatment of neurological disease, comprising a step of administering to a subject in need thereof a composition comprising, isolated stem cells; and a substance that increases the expression or activity of TIMP-1, MCP-1, GROα, and IL-6 genes or their proteins as an active ingredient.

Additionally, the present invention provides the use of isolated stem cells, and a substance for increasing the expression or activity of TIMP-1, MCP-1, GROα, and IL-6 genes or their proteins: or a composition comprising the same as an active ingredient, for the prevention, improvement, and/or treatment of neurological disease.

Additionally, the present invention provides the use of isolated stem cells; and a substance that increases the expression or activity of TIMP-1, MCP-1, GROα, and IL-6 genes or their proteins: or a composition comprising the same as an active ingredient, for the manufacture (production) of a preparation (particularly, a combination formulation or a combination drug) for the prevention, improvement, and/or treatment of neurological disease.

In one embodiment of the present invention, the substance that increases the expression or activity of the proteins may include one or more expression vectors comprising a polynucleotide encoding TIMP-1 protein: a polynucleotide encoding MCP-1 protein: a polynucleotide encoding GROα protein; and a polynucleotide encoding IL-6 protein, but is not limited thereto.

To achieve another object of the present invention, the present invention provides a pharmaceutical composition for inducing angiogenesis, comprising stem cells that overexpress TIMP-1, MCP-1, GROα, and IL-6 genes or their proteins; or a culture thereof as an active ingredient.

In one embodiment of the present invention, the culture (culture medium) may be a conditioned medium, comprising TIMP-1, MCP-1, GROα, and IL-6 proteins secreted by the stem cells, but is not limited thereto.

In one embodiment of the present invention, the pharmaceutical composition for inducing angiogenesis of the present invention may be for the prevention or treatment of cerebrovascular disease, but is not limited thereto.

In one embodiment of the present invention, the cerebrovascular disease may be one or more selected from the group consisting of cerebral infarction, cerebral ischemia, stroke, vascular dementia, cerebral hemorrhage, subarachnoid hemorrhage, and leukodystrophy, but are not limited thereto.

Therefore, the present invention provides a method for preventing, improving, and/or treating cerebrovascular disease (a method for inducing angiogenesis), comprising a step of administering to a subject in need thereof stem cells overexpressing TIMP-1, MCP-1, GROα, and IL-6 genes or their proteins, or a culture thereof.

Additionally, the present invention provides a method for preventing, improving, and/or treating cerebrovascular disease (a method for inducing angiogenesis), comprising a step of administering to a subject in need thereof a composition comprising stem cells overexpressing TIMP-1, MCP-1, GROα, and IL-6 genes or their proteins, or a culture thereof as an active ingredient.

Additionally, the present invention provides the use of stem cells overexpressing TIMP-1, MCP-1, GROα, and IL-6 genes or their proteins: or a culture thereof; or a composition comprising them as an active ingredient for the prevention, improvement, and/or treatment of cerebrovascular disease (angiogenesis induction use).

Additionally, the present invention provides the use of stem cells that overexpress TIMP-1, MCP-1, GROα, and IL-6 genes or their proteins, or a culture thereof; or a composition comprising them as an active ingredient, for the manufacture (production) of preparation for the prevention, improvement, and/or treatment of cerebrovascular disease (for the manufacture of a preparation for inducing angiogenesis).

To achieve another object of the present invention, the present invention provides a pharmaceutical composition for inducing angiogenesis, comprising isolated stem cells; and a substance that increases the expression or activity of TIMP-1, MCP-1, GROα, and IL-6 genes or their proteins as an active ingredient, as well as a pharmaceutical combination for inducing angiogenesis comprising the composition.

In one embodiment of the present invention, the substance that increases the expression or activity of the proteins may include one or more expression vectors comprising a polynucleotide encoding the TIMP-1 protein: a polynucleotide encoding the MCP-1 protein: a polynucleotide encoding the GROα protein; and a polynucleotide encoding the IL-6 protein, but is not limited thereto.

In one embodiment of the present invention, the angiogenesis-inducing pharmaceutical composition of the present invention and the pharmaceutical combination (pharmaceutical combination preparation) containing the composition may be for the prevention or treatment of cerebrovascular disease, but is not limited thereto.

Therefore, the present invention provides a method for the prevention, improvement, and/or treatment of cerebrovascular disease (angiogenesis induction method), comprising the step of administering (in particular, co-administration or combination administration) to a subject in need thereof, isolated stem cells; and a substance that increases the expression or activity of TIMP-1, MCP-1, GROα, and IL-6 genes or their proteins.

Additionally, the present invention provides a method for the prevention, improvement, and/or treatment of cerebrovascular disease (method for inducing angiogenesis), comprising a step of administering (in particular, co-administration or combination administration) to a subject in need thereof a composition comprising isolated stem cells; and a substance that increases the expression or activity of TIMP-1, MCP-1, GROα, and IL-6 genes or their proteins as the active ingredient.

Additionally, the present invention provides the use of isolated stem cells and a substance that increases the expression or activity of TIMP-1, MCP-1, GROα, and IL-6 genes or their proteins: or a composition containing the same as the active ingredient, for the prevention, improvement, and/or treatment of cerebrovascular disease (use of angiogenesis induction).

Additionally, the present invention provides the use of isolated stem cells; and a substance that increases the expression or activity of TIMP-1, MCP-1, GROα, and IL-6 genes or their proteins: or a composition containing the same as the active ingredient, for manufacturing (producing) a formulation for the prevention, improvement, and/or treatment of cerebrovascular disease (particularly, use for the manufacture of a preparation of a composition for inducing angiogenesis).

### Advantageous Effects

According to the present invention, not only is it suggested that a specific combination of markers including TIMP-1, MCP-1, GROα, and IL-6 can be used as biomarkers for selecting stem cells with the highest therapeutic (ameliorating) efficacy in neurological disease, but it is also demonstrated that stem cells with increased expression or activity levels of these factors, or the use of substances that regulate (particularly upregulate) these factors in stem cells, can result in significantly higher efficacy (potency) and efficiency in neurological therapeutic effects. Additionally, the present invention demonstrates the higher therapeutic potential of allogeneic stem cell transplantation.

### BRIEF DESCRIPTION OF THE DRAWINGS

FIG. 1 is a schematic diagram showing the process of analyzing the composition of substances secreted by neural stem cells using conditioned media obtained from human adult brain tissue (temporal lobe tissue from a donor with no lesions, type IIIa focal cortical dysplasia), through cytokine analysis and LC-MS/MS.

FIG. 2 shows the results of evaluating the neuroprotective effect of the conditioned media from neural stem cells isolated from adult human brain tissue in an in vitro stroke model induced by OGD (Oxygen Glucose Deprivation) in primary rat cortical neurons. (3 lots of conditioned media prepared using the same method were used for repeated experiments (*, p<0.05: **, p<0.01: ***, p<0.001 vs NC).)

FIG. 3a shows the results of evaluating the effects of neutralizing antibodies against either MCP-1 or TIMP-1, from a combination of the four efficacy factors of the present invention, on neuroprotective activity. After pre-incubating the neutralizing antibodies in the conditioned media of neural stem cells, the media was applied to an in vitro stroke model induced by OGD (Oxygen Glucose Deprivation). The results indicate that the neuroprotective ability was significantly reduced by treatment with the neutralizing antibodies of each factor (*, p<0.05: **, p<0.01: ***, p<0.001 vs NC: #, p<0.05: ##, p<0.01: ###, p<0.001 vs CM: $, p<0.05: $$, p<0.01: $$$, p<0.001 vs CM+ (each antibody treatment)).

FIG. 3b shows the results of evaluating the effects of neutralizing antibodies against either GROα or IL-6, from a combination of the four efficacy factors of the present invention, on neuroprotective activity. After pre-incubating the neutralizing antibodies in the conditioned media of neural stem cells, the media was applied to an in vitro stroke model induced by OGD (Oxygen Glucose Deprivation) in primary rat cortical neurons. The results indicate that neuroprotective ability was significantly reduced by treatment with the neutralizing antibodies of each factor (*, p<0.05: **, p<0.01; ***, p<0.001 vs NC: #, p<0.05: ##, p<0.01: ###, p<0.001 vs CM: $, p<0.05: $$, p<0.01; $$$, p<0.001 vs CM+ (each antibody treatment)).

FIG. 4 shows the results of evaluating the neuroprotective ability of the conditioned media from neural stem cells in an in vitro spinal cord injury model, created by treating primary rat spinal cord neuron cells with H2O2. When the conditioned media from neural stem cells was applied under damage conditions induced by reactive oxygen species (ROS), the survival rate of the neurons increased. (3 lots of conditioned media prepared using the same method were used for repeated experiments (*, p<0.05: **, p<0.01: ***, p<0.001 vs NC).)

FIG. 5a shows the results of an experiment in which neutralizing antibodies against MCP-1 or TIMP-1, respectively, or IgG were pre-incubated in neural stem cell-conditioned medium, among the four efficacy factor combinations of the present invention. After pre-incubation, this medium was applied to an in vitro spinal cord injury model, which was created by inducing ROS production through H2O2 treatment. The effects of each factor on the neuroprotective ability of the neural stem cell-conditioned medium were then compared and analyzed. As a result, the spinal cord neuroprotective ability was significantly reduced upon treatment with neutralizing antibodies against each factor (*, p<0.05: **, p<0.01: ***, p<0.001 vs IM: #, p<0.05: ##, p<0.01: ###, p<0.001 vs CM: $, p<0.05: $$, p<0.01: $$$, p<0.001 vs CM+IgG (10 μg/ml)).

FIG. 5b shows the results of an experiment in which neutralizing antibodies against GROα or IL-6, respectively, or IgG were pre-incubated in neural stem cell-conditioned medium, among the four efficacy factor combinations of the present invention. After pre-incubation, this medium was applied to an in vitro spinal cord injury model, which was created by inducing ROS production through H2O2 treatment. The effects of each factor on the neuroprotective ability of the neural stem cell-conditioned medium were then compared and analyzed. As a result, the spinal cord neuroprotective ability was significantly reduced upon treatment with neutralizing antibodies against each factor (*, p<0.05: **, p<0.01: ***, p<0.001 vs IM: #, p<0.05: ##, p<0.01: ###, p<0.001 vs CM: $, p<0.05: $$, p<0.01: $$$, p<0.001 vs CM+IgG (10 μg/ml)).

FIG. 6 is an image showing the results of a wound-healing assay performed to assess the change in vascular endothelial cell migration ability upon treatment with neural stem cell conditioned media. The assay was conducted using HUVEC (Human Umbilical Vein Endothelial Cells). The results demonstrated a significant increase in vascular endothelial cell migration ability upon treatment with the neural stem cell conditioned media (3 lots of conditioned media prepared using the same method were used for repeated experiments).

FIG. 7 is a graph quantitatively showing the results of a wound-healing assay performed to assess the change in vascular endothelial cell migration ability upon treatment with neural stem cell conditioned media, using HUVEC (Human Umbilical Vein Endothelial Cells). The results demonstrated a significant increase in vascular endothelial cell migration ability upon treatment with the neural stem cell conditioned media (3 lots of conditioned media prepared using the same method were used for repeated experiments) (*, p<0.05: **, p<0.01: ***, p<0.001 vs NC).

FIG. 8a shows the results of an experiment in which neutralizing antibodies against MCP-1 or TIMP-1 were pre-incubated in neural stem cell-conditioned medium, among the four efficacy factor combinations of the present invention. After pre-incubation, the effect of each factor on the migration ability of vascular endothelial cells (HUVEC) was confirmed by the wound-healing assay method. As a result, the migration ability of vascular endothelial cells was significantly reduced by the treatment with neutralizing antibodies for each factor (*, p<0.05: **, p<0.01: ***, p<0.001 vs NC: #, p<0.05: ##, p<0.01: ###, p<0.001 vs CM).

FIG. 8b shows the results of an experiment in which neutralizing antibodies against GROα or IL-6 were pre-incubated in neural stem cell-conditioned medium, among the four efficacy factor combinations of the present invention. After pre-incubation, the effect of each factor on the migration ability of vascular endothelial cells (HUVEC) was confirmed by the wound-healing assay method. As a result, the migration ability of vascular endothelial cells was significantly reduced by the treatment with neutralizing antibodies for each factor (*, p<0.05: **, p<0.01: ***, p<0.001 vs NC: #, p<0.05: ##, p<0.01: ###, p<0.001 vs CM).

FIG. 9a shows the results of an experiment using vascular endothelial cells (HUVEC) to assess the angiogenic ability induced by neural stem cell conditioned media, displaying the tube formation results. Treatment with the conditioned media enhanced the tube formation ability of endothelial cells (3 lots of conditioned media prepared using the same method were used for repeated experiments).

FIG. 9b is a graph showing the quantitative analysis of the tube length in an experiment assessing the angiogenic ability induced by neural stem cell conditioned media using vascular endothelial cells (HUVEC). Treatment with the conditioned media resulted in an increase in the tube formation ability of the vascular endothelial cells (3 lots of conditioned media prepared using the same method were used for repeated experiments (*, p<0.05: **, p<0.01: ***, p<0.001 vs NC)).

FIG. 10a shows the results of an experiment in which neutralizing antibodies against MCP-1 or TIMP-1 were pre-incubated in neural stem cell-conditioned medium, among the four efficacy factor combinations of the present invention. After pre-incubation, the effect of each factor on the angiogenic ability was confirmed by treating vascular endothelial cells (HUVEC). The tube-forming ability was significantly reduced by treatment with a neutralizing antibody against TIMP-1 (*, p<0.05: **, p<0.01: ***, p<0.001 vs NC: #, p<0.05: ##, p<0.01: ###, p<0.001 vs CM).

FIG. 10b shows the results of an experiment in which neutralizing antibodies against GROα or IL-6 were pre-incubated in neural stem cell-conditioned medium, among the four efficacy factor combinations of the present invention. After pre-incubation, the effect of each factor on the angiogenic ability was confirmed by treating vascular endothelial cells (HUVEC). The tube-forming ability was significantly reduced by the neutralizing antibody treatment against each factors (*, p<0.05: **, p<0.01: ***, p<0.001 vs NC: #, p<0.05: ##, p<0.01: ###, p<0.001 vs CM).

FIG. 11a is a graph showing the results of a behavioral test BBB score test (Basso, Beattie and Bresnahan score test) conducted on an in vivo spinal cord injury animal model. The test was performed at one-week intervals to assess the improvement in behavioral deficits following the injection (transplantation) of highly potent neural stem cells selected according to the present invention (G1, Vehicle (DMEM F12-phenol free); G2, 3×10⁶ cells/per animal: G3, 1×10⁶ cells/per animal: *, G1 (n=42) vs G2 (n=41): #, G1 vs G3 (n=42)).

FIG. 11b is a graph showing the results of a rota-rod test conducted on an in vivo spinal cord injury animal model. The test was performed at one-week intervals to assess the improvement in behavioral deficits following the injection (transplantation) of highly potent neural stem cells selected according to the present invention (*, G1 (n=42) vs G2 (n=41): #, G1 vs G3 (n=42)).

FIG. 11c is a graph showing the results of the von Frey test conducted on an in vivo spinal cord injury animal model. The test was performed at one-week intervals to evaluate the improvement in neuropathic pain following the injection (transplantation) of highly potent neural stem cells selected according to the present invention (*, G1 (n=42) vs G2 (n=41): #, G1 vs G3 (n=42)).

FIG. 12a is a schematic timeline showing a series of experiments conducted to confirm the therapeutic effect of highly potent neural stem cells selected according to the present invention, using an in vivo ischemic stroke animal model (middle cerebral artery occlusion model, MCAO).

FIG. 12b is an image showing the results of TCC staining performed on the brain of an experimental animal to evaluate the effect of improving cerebral infarction by injection (transplantation) of high-efficacy neural stem cells selected according to the present invention in an in vivo ischemic stroke animal model (middle cerebral artery occlusion model, MCAO).

FIG. 12c is a graph showing the quantitative measurement of the size (volume) of the brain infarction area in experimental animals after performing TCC staining performed on the brain of an experimental animal to evaluate the effect of improving cerebral infarction by injection (transplantation) of high-efficacy neural stem cells selected according to the present invention in an in vivo ischemic stroke animal model (middle cerebral artery occlusion model, MCAO). In the experiment, the animal groups were divided into a control group (without injury), a damage control group after inducing stroke (HBSS), and a neural stem cell transplantation group (NSC, 5×10⁵ cells/per animal). The results of the brain infarction improvement test showed that in animals that received intraventricular administration of stem cells (NSC), brain infarction was significantly reduced compared to animals that did not receive stem cells (HBSS) (*, p<0.05; **, p<0.01).

FIG. 13a is a graph showing the results of the rota-rod test conducted at weekly intervals to evaluate the improvement of motor disabilities following the administration (transplantation) of highly potent neural stem cells selected according to the present invention in an in vivo ischemic stroke animal model (middle cerebral artery occlusion model, MCAO) (*, p<0.05 compared to Control: #, p<0.05 compared to HBSS: ##, p<0.01 compared to HBSS: ###, p<0.001 compared to HBSS).

FIG. 13b is a graph showing the results of the passive avoidance test conducted at weekly intervals to evaluate the improvement of cognitive and memory functions following the administration (transplantation) of highly potent neural stem cells selected according to the present invention in an in vivo ischemic stroke animal model (middle cerebral artery occlusion model, MCAO) (*, p<0.05 compared to Control: #, p<0.05 compared to HBSS: ##, p<0.01 compared to HBSS: ###, p<0.001 compared to HBSS).

FIG. 13c is a graph showing the results of the adhesive removal test conducted at weekly intervals to evaluate the improvement of sensory and motor functions following the administration (transplantation) of highly potent neural stem cells selected according to the present invention in an in vivo ischemic stroke animal model (middle cerebral artery occlusion model, MCAO) (*, p<0.05 compared to Control: #, p<0.05 compared to HBSS: ##, p<0.01 compared to HBSS: ###, p<0.001 compared to HBSS).

FIG. 13d is a graph showing the results of the adhesive removal test conducted at weekly intervals to evaluate the improvement of sensory and motor functions following the administration (transplantation) of highly potent neural stem cells selected according to the present invention in an in vivo ischemic stroke animal model (middle cerebral artery occlusion model, MCAO) (*, p<0.05 compared to Control: #, p<0.05 compared to HBSS: ##, p<0.01 compared to HBSS: ###, p<0.001 compared to HBSS).

FIG. 14a shows the selected tissue regions used to observe the stroke therapeutic effects of highly potent neural stem cells selected according to the present invention injected (transplanted) in an in vivo ischemic stroke animal model (middle cerebral artery occlusion model, MCAO). The damaged ipsilateral and non-damaged contralateral regions were observed on average across three parts. The ipsilateral and contralateral regions referred to in the present specification and figures correspond to the regions indicated in FIG. 14a.

FIG. 14b shows the result of NeuN staining performed to compare the number of neurons in the damaged region after the injection (grafting) of the selected highly potent neural stem cells selected according to the present invention in an in vivo ischemic stroke animal model (middle cerebral artery occlusion model, MCAO) (control, control group: HBSS, damage control group: NSC, test group with neural stem cell graft).

FIG. 14c shows a graph quantifying the number of neurons in each experimental group, following NeuN staining performed to compare the number of neurons in the damaged area after the injection (grafting) of highly potent neural stem cells selected according to the present invention, in an in vivo ischemic stroke animal model (middle cerebral artery occlusion model, MCAO). It was confirmed that the survival of neural cells in the damaged brain tissue increased following the administration of highly potent neural stem cells according to the present invention (control, control group; HBSS, damage control group: NSC, experimental group with neural stem cell grafting, *, p<0.05: **, p<0.01: ***, p<0.001).

FIG. 15a shows the result image of immunohistochemical staining with GFAP antibody to compare the degree of reactive gliosis in the damaged area after the administration (grafting) of highly potent neural stem cells selected according to the present invention in an in vivo ischemic stroke animal model (middle cerebral artery occlusion model, MCAO).

FIG. 15b shows the graph representing the quantitative measurement of astrocytes in each experimental group after performing immunohistochemical staining with GFAP antibody to compare the degree of reactive gliosis in the damaged area following the administration (grafting) of highly potent neural stem cells selected according to the present invention in an in vivo ischemic stroke animal model (middle cerebral artery occlusion model, MCAO) (*, p<0.05: **, p<0.01: ***, p<0.001).

FIG. 15c shows the image obtained after performing immunohistochemical staining with Iba1 antibody to compare the levels of reactive microglia in the damaged area following the administration (grafting) of highly potent neural stem cells selected according to the present invention in an in vivo ischemic stroke animal model (middle cerebral artery occlusion model, MCAO).

FIG. 15d shows a graph quantifying the microglia in each experimental group, after performing immunohistochemical staining with Iba1 antibody to compare the levels of reactive microglia in the damaged area following the administration (grafting) of highly potent neural stem cells selected according to the present invention in an in vivo ischemic stroke animal model (middle cerebral artery occlusion model, MCAO). (*, p<0.05: **, p<0.01: ***, p<0.001).

FIG. 16a shows an image of the Western blot results performed on brain tissue to confirm the expression levels of the proteins related to cell survival and apoptosis, such as BAX, Bcl2, and cleaved caspase-3, following the administration (grafting) of highly potent neural stem cells selected according to the present invention in an in vivo ischemic stroke animal model (middle cerebral artery occlusion model, MCAO).

FIG. 16b is a graph showing the quantitative results of Western blot analysis performed on brain tissue to confirm the expression levels of proteins related to cell survival and apoptosis, such as BAX, Bcl2, and cleaved caspase-3, following the administration (grafting) of highly potent neural stem cells selected according to the present invention in an in vivo ischemic stroke animal model (middle cerebral artery occlusion model, MCAO) (*, p<0.05: **, p<0.01, ***, p<0.001).

FIG. 17a is an image showing the results of CD31 immunofluorescence staining (vascular staining) performed on the cortical region of the brain to assess the recovery of brain blood vessels following the administration (grafting) of highly potent neural stem cells selected according to the present invention in an in vivo ischemic stroke animal model (middle cerebral artery occlusion model, MCAO).

FIG. 17b is a graph showing the quantitative measurement of angiogenesis (regeneration) levels in each experimental group, after performing CD31 immunofluorescence staining (vascular staining) on the cortical region of the damaged area to assess the recovery of brain blood vessels following the administration (grafting) of highly potent neural stem cells selected according to the present invention in an in vivo ischemic stroke animal model (middle cerebral artery occlusion model, MCAO) (*, p<0.05; **, p<0.01, ***, p<0.001).

FIG. 18 is a schematic diagram summarizing, from various aspects, the specific combination of four factors, MCP-1, TIMP-1, GROα, and IL-6, and the excellent effect of highly effective neural stem cells selected based on these factors on the treatment of neurological disease in the present invention using neural stem cells.

### BEST MODE

### Definition

Unless otherwise defined, all technical and scientific terms used in this specification have the same meaning commonly understood by those skilled in the art.

In the present invention, the term ‘neurological disease’ refers to diseases arising from problems in the nervous system, including the brain, spinal cord, nerves, and the like, and may also encompass certain psychiatric disorders. The neurological disease may include diseases of the brain nerves, central nervous system, or peripheral nervous system. In the present invention, the neurological disease is not specifically limited in type as long as they are diseases requiring neural (nerve cell) regeneration. However, they may include any one or more selected from the group consisting of ischemic brain disease, degenerative neurological disease, neurological injury diseases, peripheral nerve disorders, and traumatic brain injuries.

In the present invention, the term ‘ischemia’ refers to a state where the supply of blood is blocked, resulting in a deficient condition that leads to necrosis in the affected tissue area. The ischemic brain diseases includes, but are not limited to, those known in the art as conditions caused by cerebral ischemia. For example, these may include stroke, cerebral infarction, cerebral ischemia, cerebral embolism, cerebral thrombosis, transient ischemic attack (TIA), head trauma, cerebrovascular metabolic disorders, brain coma, intracerebral hemorrhage, neonatal intraventricular hemorrhage (IVH), cerebral arteriosclerosis, hypoxic brain injury, and neonatal hypoxic-ischemic brain injury, any one or more of which may be selected from the group.

The neurodegenerative diseases include, but are not limited to, those known in the art as degenerative neurological disease. For example, these may include dementia, Alzheimer's disease, Parkinson's disease, progressive supranuclear palsy, multiple system atrophy, olivopontocerebellar atrophy (OPCA), Shy-Drager syndrome, striatonigral degeneration, Huntington's disease, amyotrophic lateral sclerosis (ALS), essential tremor, corticobasal ganglionic degeneration, diffuse Lewy body disease, Parkinson-ALS-dementia complex of Guam, and Pick's disease, any one or more of which may be selected from the group.

The neurological damage disorders include, but are not limited to, those that may be selected from the group consisting of epilepsy, spinal cord injury diseases, behavioral disorders, developmental disorders, intellectual disabilities, Down syndrome, and schizophrenia, any one or more of which may be selected.

The term ‘neuroregeneration’ used throughout this specification refers to the process by which nerves regenerate or re-grow. In this specification, neuroregeneration and neurogenesis may be used interchangeably. In the present invention, the term ‘neurogenesis’ refers to the process by which new neurons are produced, as is generally known in the art.

In the present invention, the term ‘protein’ is used interchangeably with “polypeptide,” and refers to a polymer of amino acid residues, as typically found in naturally occurring proteins.

In the present invention, the TIMP-1 (TIMP Metallopeptidase Inhibitor 1) protein refers to a protein or polypeptide known in the art as TIMP-1, and its specific origin and sequence (amino acid sequence composition) are not particularly limited. For example, in the present invention, TIMP-1 is derived from Homo sapiens and includes those known in the art, such as NCBI (GenBank) Accession No. NP_003245.1 (sequence number 1) or UniProtKB/Swiss-Prot Accession No. Q6FGX5. In the present invention, the TIMP-1 includes functional equivalents thereof.

In the present invention, the MCP-1 (Monocyte Chemoattractant Protein-1) protein, if known as the MCP-1 protein or polypeptide in the art, is not specifically limited by its origin and sequence (amino acid sequence composition). For example, the MCP-1 in the present invention is derived from human (Homo sapiens) and includes those disclosed in NCBI (Genbank) Accession Nos. AAB20651.1, NP_002973.1 (Sequence No. 2), or UniProtKB/Swiss-Prot Accession No. P13500.1. In the present invention, the MCP-1 includes functional equivalents thereof.

In the present invention, the GROα (Growth-Regulated Oncogene alpha: chemokine (C—X—C motif) ligand 1 (CXCL1)) protein, if known as the GROα protein or polypeptide in the art, is not specifically limited by its origin and sequence (amino acid sequence composition). For example, the GROα in the present invention is derived from human (Homo sapiens) and includes those disclosed in NCBI (Genbank) Accession No. NP_001502.1 (Sequence No. 3), or UniProtKB/Swiss-Prot Accession No. P09341. In the present invention, the GROα includes functional equivalents thereof.

In the present invention, the IL-6 (Interleukin 6) protein, if known as the IL-6 protein or polypeptide in the art, is not specifically limited by its origin and sequence (amino acid sequence composition). For example, the IL-6 in the present invention is derived from human (Homo sapiens) and includes those disclosed in NCBI (Genbank) Accession No. NP_000591.1 (Sequence No. 4), or UniProtKB/Swiss-Prot Accession No. P05231. In the present invention, the IL-6 includes functional equivalents thereof.

The functional equivalent referred to herein denotes a polypeptide having at least 70% or more, preferably 80% or more, and more preferably 90% or more sequence homology (i.e., identity) with the amino acid sequences constituting the known TIMP-1, MCP-1, GROα, or IL-6 proteins, which are preferably exemplified by the amino acid sequences represented by Sequence No. 1, Sequence No. 2, Sequence No. 3, or Sequence No. 4, respectively. For example, it includes polypeptides having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence homology and exhibiting substantially the same physiological activity as the parent known proteins. Here, the term ‘substantially the same physiological activity’ refers to the regulation of stem cell activity for the prevention, improvement, and/or treatment of neurological disease. Preferably, in the present invention, the functional equivalents of TIMP-1, MCP-1, GROα, or IL-6 may be generated as a result of addition, substitution, or deletion of a portion of the amino acid sequences of SEQ ID NO: 1, SEQ ID NO: 2, SEQ ID NO: 3, or SEQ ID NO: 4. The substitution of amino acids described above is preferably a conservative substitution. Examples of conservative substitutions of naturally occurring amino acids are as follows: Aliphatic amino acids (Gly, Ala, Pro), hydrophobic amino acids (Ile, Leu, Val), aromatic amino acids (Phe, Tyr, Trp), acidic amino acids (Asp, Glu), basic amino acids (His, Lys, Arg, Gln, Asn), and sulfur-containing amino acids (Cys, Met). Additionally, the functional equivalents include variants in which some amino acids are deleted in the amino acid sequence of TIMP-1, MCP-1, GROα, or IL-6 proteins. The deletion or substitution of the amino acids is preferably located in regions not directly related to the physiological activity of TIMP-1, MCP-1, GROα, or IL-6. Additionally, the functional equivalents include variants in which several amino acids are added to the N- or C-terminus or within the amino acid sequences of TIMP-1, MCP-1, GROα, or IL-6. Additionally, the scope of the functional equivalents of the present invention includes polypeptide derivatives in which certain chemical structures of the polypeptide are modified, while maintaining the fundamental framework and physiological activity of TIMP-1, MCP-1, GROα, or IL-6. For example, structural modifications aimed at altering the stability, storage capacity, volatility, or solubility of the protein are included within this scope.

In this specification, sequence homology and identity are defined as the percentage of identical matched residues (amino acid residues or nucleotides) between the original sequence (for example, SEQ ID NO: 1, SEQ ID NO: 2, SEQ ID NO: 3, or SEQ ID NO: 4 for amino acid sequences; or SEQ ID NO: 5, SEQ ID NO: 6, SEQ ID NO: 7, or SEQ ID NO: 8 for nucleotide sequences) and the candidate sequence, after aligning the sequences and introducing gaps. If necessary, conservative substitutions are not considered as part of sequence identity in order to achieve the maximum percentage of sequence homology. Additionally, for determining protein sequence homology or identity, extensions, deletions, or insertions at the N-terminus, C-terminus, or within the internal sequence are not considered as sequences affecting sequence homology or identity. Additionally, the sequence identity can be determined using common standard methods employed to compare similar regions of the amino acid sequences of two polypeptides. Computer programs such as BLAST or FASTA align two polypeptides so that each amino acid is optimally matched (either along the full length of one or both sequences, or along predicted portions of one or both sequences). The programs provide a default opening penalty and a default gap penalty, and may be used in conjunction with scoring matrices such as PAM250 (a standard scoring matrix; Dayhoff et al., in Atlas of Protein Sequence and Structure, vol 5, supp 3, 1978), which can be linked with the computer programs. For example, percentage identity can be calculated as follows. The total number of identical matches is multiplied by 100, and then divided by the sum of the length of the longer sequence within the matched span and the number of gaps introduced into the longer sequence to align the two sequences.

In the present invention, the term ‘nucleic acid,’ ‘DNA sequence,’ ‘RNA sequence,’ or ‘polynucleotide’ refers to deoxyribonucleotides or ribonucleotides in single-stranded or double-stranded form. Unless otherwise restricted, the term ‘nucleic acid’ also includes known analogs of naturally occurring nucleotides that hybridize with the nucleic acid in a manner similar to naturally occurring nucleotides.

In the present invention, the term ‘polynucleotide encoding TIMP-1’ may refer to a nucleotide (nucleic acid) sequence that encodes the amino acid sequence designated as SEQ ID NO: 1 or an amino acid sequence having at least 70% sequence identity to it. The nucleic acid may include DNA, cDNA, and RNA sequences. In other words, the nucleic acid encoding TIMP-1 may have a nucleotide sequence encoding the amino acid sequence of SEQ ID NO: 1 or an amino acid sequence having at least 70% sequence identity thereto, or a complementary nucleotide sequence thereof, including DNA, cDNA, and RNA (for example, TIMP-1 mRNA). Preferably, it may include a nucleotide sequence represented by NCBI (Genbank) Accession No. NM_003254.3 (SEQ ID NO: 5), and more preferably, it may consist of the nucleotide sequence represented by SEQ ID NO: 5.

In the present invention, the term ‘polynucleotide encoding MCP-1’ may refer to a nucleotide (nucleic acid) sequence that encodes the amino acid sequence designated as SEQ ID NO: 2 or an amino acid sequence having at least 70% sequence identity to it. The nucleic acid may include DNA, cDNA, and RNA sequences. In other words, the nucleic acid encoding MCP-1 may have a nucleotide sequence encoding the amino acid sequence of SEQ ID NO: 2 or an amino acid sequence having at least 70% sequence identity thereto, or a complementary nucleotide sequence thereof, including DNA, cDNA, and RNA (for example, MCP-1 mRNA). Preferably, it may include a nucleotide sequence as represented by NCBI (GenBank) Accession No. NM_002982.3 (SEQ ID NO: 6), and more preferably, it may consist of the nucleotide sequence represented by SEQ ID NO: 6.

In the present invention, the term ‘polynucleotide encoding GROα’ may refer to a nucleotide (nucleic acid) sequence that encodes the amino acid sequence designated as SEQ ID NO: 3 or an amino acid sequence having at least 70% sequence identity to it. The nucleic acid may include DNA, cDNA, and RNA sequences. In other words, the nucleic acid encoding GROα may have a nucleotide sequence encoding the amino acid sequence of SEQ ID NO: 3 or an amino acid sequence having at least 70% sequence identity thereto, or a complementary nucleotide sequence thereof, including DNA, cDNA, and RNA (for example, GROα mRNA). Preferably, it may include a nucleotide sequence represented by NCBI (Genbank) Accession No. NM_001511.4 (SEQ ID NO: 7), and more preferably, it may consist of the nucleotide sequence represented by SEQ ID NO: 7.

In the present invention, the term ‘polynucleotide encoding IL-6’ may refer to a nucleotide (nucleic acid) sequence that encodes the amino acid sequence designated as SEQ ID NO: 4 or an amino acid sequence having at least 70% sequence identity to it. The nucleic acid may include DNA, cDNA, and RNA sequences. In other words, the nucleic acid encoding IL-6 may have a nucleotide sequence encoding the amino acid sequence of SEQ ID NO: 4 or an amino acid sequence having at least 70% sequence identity thereto, or a complementary nucleotide sequence thereof, including DNA, cDNA, and RNA (for example, IL-6 mRNA). Preferably, it may include a nucleotide sequence represented by NCBI (Genbank) Accession No. NM_000600.5 (SEQ ID NO: 8), and more preferably, it may consist of the nucleotide sequence represented by SEQ ID NO: 8.

In the present invention, the polynucleotide encoding the expression-target protein may undergo various modifications within the coding region without altering the amino acid sequence of the protein expressed from the coding region, due to the degeneracy of codons or by considering the codons preferred by the organism in which the protein is to be expressed. Additionally, modifications may occur in regions outside the coding region, provided they do not affect the expression of the gene. Such modified genes will also be understood to fall within the scope of the present invention by those skilled in the art. In other words, the polynucleotide of the present invention may be modified by substitution, deletion, insertion, or a combination thereof of one or more nucleotides, as long as it encodes a protein having equivalent activity, and such modifications are also included within the scope of the present invention.

In the present invention, the term ‘analog’ refers to a substance that is structurally similar to a reference molecule, but where specific substituents of the reference molecule are replaced by substitutions, thereby modifying the target or regulatory mechanism of the substance. Compared to the reference molecule, the analog has the same, similar, or enhanced utility, as would be expected by those skilled in the art. The synthesis and screening of analogs to identify compounds with improved properties (e.g., higher binding affinity for target molecules) are methods well known in the field of pharmacochemical sciences.

In the present invention, the term ‘homologues’ refers to proteins and/or protein sequences that are derived naturally or artificially from a common ancestral protein or protein sequence. Similarly, nucleic acids and/or nucleic acid sequences are considered homologous when they are derived naturally or artificially from a common ancestral nucleic acid or nucleic acid sequence.

In the present invention, the term ‘contacting’ refers to its general meaning, which involves combining two or more agents (e.g., two polypeptides) or combining an agent with a cell (e.g., a protein and a cell). Contacting may occur in vitro. For example, it refers to combining two or more compositions in a test tube or other containers, or combining a test composition with cells or cell lysates. Additionally, contacting may occur in cells or in situ. For example, contacting may involve coexpressing recombinant polynucleotides encoding two polypeptides within a cell, thereby contacting the two polypeptides in the cell or cell lysate.

In the present invention, the term ‘test substance’ or ‘test agent’ refers to any substance, molecule, element, compound, entity, or a combination thereof. For example, without being limited thereto, it includes proteins, polypeptides, small organic molecules, polysaccharides, polynucleotides, and the like. Additionally, it may be a natural product, a synthetic compound, a chemical compound, or a combination of two or more substances. Unless otherwise specified, the formulation, substance, and compound may be used interchangeably.

More specifically, the test agents that can be screened using the screening method of the present invention include polypeptides, antibodies, beta-turn mimetics, polysaccharides, phospholipids, hormones, prostaglandins, steroids, aromatic compounds, heterocyclic compounds, benzodiazepines, oligomeric N-substituted glycines, oligocarbamates, saccharides, fatty acids, purines, pyrimidines, or derivatives, structural analogs, or combinations thereof. Some test agents may be synthetic substances (synthetic compounds), while other test agents may be natural substances (natural compounds, plant extracts, etc.). The test agents may be obtained from a wide and diverse range of sources, including libraries of synthetic or natural compounds. Combinatorial libraries may be generated with various types of compounds that can be synthesized through a step-by-step process. Many compounds in combinatorial libraries may be prepared using the ESL (encoded synthetic libraries) method (WO 95/12608, WO 93/06121, WO 94/08051, WO 95/395503, and WO 95/30642). Peptide libraries may be prepared using the phage display method (WO 91/18980). Libraries of natural compounds in the form of extracts from bacteria, fungi, plants, and animals may be obtained from commercial sources or collected from the field. Known pharmacological agents may be subjected to directed or random chemical modifications, such as acylation, alkylation, esterification, or amidification reactions, to produce structural analogs.

The test agent may be a naturally occurring protein or a fragment thereof. Such test agents may be obtained from a natural source, for example, from cell or tissue lysates. The library of polypeptide agents may be obtained, for example, by conventional methods or from commercially available cDNA libraries. The test agent may be a peptide, for example, a peptide having about 5 to 30 amino acids, preferably about 5 to 20 amino acids, and more preferably about 7 to 15 amino acids. The peptide may be a naturally occurring protein, a random peptide, or a fragment of a “biased” random peptide.

Additionally, the test agent may be a nucleic acid. Additionally, the nucleic acid test agent may be a naturally occurring nucleic acid, a random nucleic acid, or a “biased” random nucleic acid. Additionally, fragments of prokaryotic or eukaryotic genomes may be used in a manner similar to that described above.

Additionally, the test agent may be a small molecule (e.g., a molecule with a molecular weight of approximately 1,000 or less). High throughput assays may be applied for screening small molecule regulatory agents. Many assays are useful for the screening.

The library of test agents screened in the method of the present invention may be produced based on structural studies of full-length proteins or fragments (fragment polypeptides) of TIMP-1, MCP-1, GROα, or IL-6, or their analogs. Such structural studies enable the identification of test agents that are likely to bind or interact with the aforementioned proteins. The three-dimensional structure of the aforementioned proteins can be studied using various methods, such as crystallographic structure and molecular modeling. The protein structure study method using X-ray crystallography is well known in the art. Computer modeling of protein structures provides an alternative means for the design of test agents for screening. Molecular modeling methods are known in the art and are disclosed, for example, in the following references: U.S. Pat. Nos. 5,612,894 and 5,583,973. Additionally, protein structures can be determined by neutron diffraction and nuclear magnetic resonance (NMR).

In the present invention, the term ‘primer’ refers to a single-stranded oligonucleotide that can act as a starting point for template-directed DNA synthesis under appropriate conditions in a suitable buffer (for example, four different nucleoside triphosphates and a polymerase such as DNA, RNA polymerase, or reverse transcriptase) and at an appropriate temperature. The appropriate length of the primer may vary depending on the intended use, but it is typically between 15 and 30 nucleotides. The term “substantially complementary” used in relation to primers means that the primer is sufficiently complementary to the template nucleic acid sequence to selectively hybridize under certain annealing conditions, and the annealed primer is extended by a polymerase to generate the complementary strand of the template. Therefore, the term “substantially complementary” has a different meaning from the term “perfectly complementary” or related terms. Short primer molecules generally require a lower temperature to form a stable hybrid with the template. The primer sequence does not need to be perfectly complementary to the template, but it must be sufficiently complementary (substantially complementary) to hybridize with the template. The primer hybridizes to the target DNA containing a polymorphic region and initiates amplification of the allele form that exhibits perfect homology. The primer is used in combination with a second primer that hybridizes to the opposite strand. The amplification results in the products being amplified from both primers, indicating the presence of a specific allele. The primers of the present invention include polynucleotide fragments used in ligase chain reaction (LCR).

In the present invention, the term “probe” refers to a typically labeled oligonucleotide that has a sequence complementary to a target nucleic acid sequence and forms a double structure through complementary base pairing. The probe will typically include a “hybridization region” corresponding to a region of the target sequence, preferably consisting of at least 30 nucleotides, and in some cases, more than 50 nucleotides. The term “corresponding” refers to something that is either identical to or complementary to the designated nucleic acid. The probe may, preferably, not contain sequences complementary to the sequence(s) used for preparing the PCR. Generally, the 3′ end of the probe will be “blocked” to prevent its integration into the primer extension product. “Blocking” can be achieved by adding a nucleotide to the 3′ hydroxyl of the base, which can fulfill dual purposes: preventing integration into the primer extension product and acting as a label for subsequent detection or capture of the labeled nucleic acid, using a non-complementary base or a chemical moiety such as biotin, phosphate groups, or a fluorophore, depending on the selected moiety. Blocking can be achieved by removing the 3′—OH group or by using a nucleotide without a 3′—OH group, such as a dideoxynucleotide.

In the present invention, the term ‘TIMP-1 antibody’ or ‘antibody against TIMP-1’ refers to a specific protein molecule directed against the antigenic site of TIMP-1. For the purposes of the present invention, the antibody refers to an antibody that specifically binds to the TIMP-1 protein, and it includes polyclonal antibodies, monoclonal antibodies, and recombinant antibodies. For the purposes of the present invention, it may be preferable for the antibody to be a monoclonal antibody, which is a population of antibodies having substantially identical amino acid sequences in their heavy and light chains. In the present invention, when ‘MCP-1 antibody,’ ‘GROα antibody,’ or ‘IL-6 antibody’ is mentioned or referred to, the meaning thereof will be clearly understood by those skilled in the art in accordance with the description pertaining to the ‘TIMP-1 antibody.’

The production of antibodies against TIMP-1, MCP-1, GROα, or IL-6, as described above, can be readily achieved using techniques well known in the art. Polyclonal antibodies can be produced using methods well known in the art, wherein the TIMP-1, MCP-1, GROα, or IL-6 protein antigen is injected into an animal, and serum containing the antibodies is obtained by collecting blood from the animal. Such polyclonal antibodies can be produced from host animals of any species, including goats, rabbits, sheep, monkeys, horses, pigs, cattle, and dogs.

Monoclonal antibodies can be produced using the hybridoma method, which is widely known in the art, or by employing phage antibody library techniques.

Additionally, in the present invention, the term antibody includes not only the complete form having two full-length light chains and two full-length heavy chains, but also functional fragments of the antibody molecule. The functional fragment of the antibody molecule refers to a fragment that retains at least the antigen-binding functionality, and preferably, the fragment retains at least 50%, 60%, 70%, 80%, 90%, 95%, or 100% or more of the binding affinity of the parent antibody for TIMP-1, MCP-1, GROα, or IL-6. Specifically, the functional fragment may be in the form of Fab, F(ab)2, Fab′, F(ab′)2, Fv, diabody, scFv, or other similar formats. Fab (fragment antigen-binding) is an antigen-binding fragment of an antibody, consisting of one variable domain and one constant domain from both the heavy chain and the light chain. F(ab′)2 is a fragment generated by the pepsin digestion of an antibody, consisting of two Fab fragments connected by a disulfide bond at the hinge region of the heavy chain. F(ab′) is a monomeric antibody fragment derived by reducing the disulfide bond of the F(ab′)2 fragment, where a hinge region of the heavy chain is added to the Fab fragment. Fv (variable fragment) is an antibody fragment consisting solely of the variable regions of both the heavy and light chains. scFv (single chain variable fragment) is a recombinant antibody fragment in which the variable region of the heavy chain (VH) and the variable region of the light chain (VL) are connected by a flexible peptide linker. A diabody is a fragment in which the VH and VL of an scFv are connected by a very short linker, preventing them from associating with each other. Instead, they pair with the VH and VL of another scFv of the same form, thereby forming a dimer.

The antibodies applicable in the present invention are not limited thereto, but may be selected from the group consisting of IgG, IgA, IgM, IgE, and IgD, and preferably, may be an IgG antibody.

In the present invention, the term ‘expression’ refers to the production of a protein or nucleic acid in a cell.

In the present invention, the term ‘host cell’ refers to a prokaryotic or eukaryotic cell that contains heterologous DNA introduced into the cell by any means (e.g., electroporation, calcium phosphate precipitation, microinjection, transformation, viral infection, etc.).

The present invention will be described in detail below.

The inventors of the present invention, through research into functions other than the differentiation into neurons, which is known as a fundamental function of neural stem cells, have identified for the first time that TIMP-1, MCP-1, GRO-α, and IL-6 play key roles and directly contribute to the therapeutic activity in treating neurological disease among neural stem cells derived from human brain tissue.

Accordingly, the inventors have enabled the selection of stem cells with the most superior therapeutic (improving) efficacy for neurological disease by utilizing a specific combination of markers, TIMP-1, MCP-1, GROα, and IL-6. Using these selected stem cells, they have confirmed significant therapeutic effects in an in vivo neurological disease animal model.

Additionally, by directly utilizing stem cells with increased expression or activity levels of TIMP-1, MCP-1, GROα, and IL-6 factors, or by using substances that regulate (particularly upregulate) the expression or activity of these factors in stem cells, it is possible to more significantly and efficiently treat neurological disease with high efficacy (high performance) and high efficiency.

Accordingly, the present invention provides a method for selecting high-efficacy stem cells for the treatment of neurological disease, which includes the step of measuring the protein or gene expression levels of TIMP-1 (TIMP Metallopeptidase Inhibitor 1), MCP-1 (Monocyte Chemoattractant Protein-1), GROα (Growth-Regulated Oncogene alpha), and IL-6 (Interleukin 6).

In the method for selecting high-efficacy stem cells for the treatment of neurological disease, the proteins or genes of TIMP-1, MCP-1, GROα, and IL-6 are characterized by being highly expressed in the high-efficacy stem cells for neurological disease treatment.

In the present invention, the ‘stem cells’ or ‘isolated stem cells’ prior to selection are not particularly limited in terms of their specific origin or type, provided that they are known in the art to be used for the treatment of neurological disease. However, preferably, they refer to stem cells possessing the ability to differentiate into neural cells. Not limited thereto, in the present invention, the stem cells are preferably selected from the group consisting of mesenchymal stem cells, adult stem cells, dedifferentiated stem cells, embryonic stem cells, induced pluripotent stem cells, neural stem cells, tissue-derived stem cells, and any combination of two or more thereof.

The ‘stem cells’ or ‘isolated stem cells’ prior to selection in the present invention can be isolated and obtained according to methods known in the art. Examples of such known methods, without being limited thereto, include density gradient fractionation (especially density gradient centrifugation), immunoselection, layer separation culture methods, differential adhesion separation, and fluorescence-activated cell sorting (FACS).

In a preferred embodiment of the present invention, the stem cells of the present invention (in particular, neural stem cells) may be derived from human brain or human nervous tissue (central or peripheral nervous tissue), but is not limited thereto. As described in an embodiment of the present application, the present inventors have solved ethical issues associated with the use of embryonic stem cells while easily providing transplantable human adult neural stem cells by isolating and culturing neural stem cells isolated from the temporal lobe tissue of a localized cortical dysplasia IIIa-type donor (from a non-lesioned area excised during brain surgery of the donor with localized cortical dysplasia IIIa). In the present invention, human adult neural stem cells isolated from the brain tissue of a localized cortical dysplasia IIIa-type donor are capable of autologous and allogeneic transplantation.

In the present invention, the neural stem cells may include neural progenitor cells.

In the present invention, the term ‘culture of stem cells’ refers to the product obtained after culturing the stem cells according to known stem cell culture methods, and its specific form, such as solid or liquid, is not particularly limited. In one embodiment of the present invention, the culture may be used in the form of a liquid culture medium.

If the culture contains the proteins TIMP-1, MCP-1, GROα, and IL-6 secreted by the stem cells, the composition of the culture is not particularly limited. In the present invention, the culture encompasses both compositions in which the stem cells are included in the culture and compositions in which the stem cells are not included in the culture.

In one embodiment of the present invention, the culture may not include stem cells that overexpress the genes or proteins of TIMP-1, MCP-1, GROα, and IL-6. That is, in the present invention, the culture may be a conditioned medium in which stem cells overexpressing the genes or proteins of TIMP-1, MCP-1, GROα, and IL-6 are cultured. In the present invention, the conditioned medium refers to the secretion (secretome) produced by stem cells overexpressing the genes or proteins of TIMP-1, MCP-1, GROα, and IL-6, which is obtained by culturing the stem cells in a conventional basic medium known in the art (preferably, a medium used for mammalian cell culture, and more preferably, a commonly used stem cell culture medium). The stem cells themselves, which overexpress the genes or proteins of TIMP-1, MCP-1, GROα, and IL-6, are removed, and only the medium is collected.

In the present invention, the ‘basic medium’ refers to a mixture containing essential components such as sugars, amino acids, water, and other necessary substances for cell survival. The basic medium of the present invention can be either synthetically prepared for use or commercially available medium. Commercially available media include, but are not limited to, DMEM (Dulbecco's Modified Eagle's Medium), low-glucose DMEM, MEM (Minimal Essential Medium), MEM-alpha medium, BME (Basal Medium Eagle), IMEM (Improved Minimum Essential Medium), K-SFM (Keratinocyte Serum-Free Medium), EGM-2 (Endothelial Cell Growth Medium 2), RPMI 1640, F-10, F-12, Ham's F-12 medium, α-MEM (α-Minimal Essential Medium), G-MEM (Glasgow's Minimal Essential Medium), McCoy's 5A medium, Eagle's basal medium (BME, Basal Medium Eagle), CMRL medium, CMRL 1066 medium, Glasgow Minimum Essential Medium (GMEM), Leibovitz's L-15 medium, KSB-3 basal medium, KSB-3 Complete medium, and Iscove's Modified Dulbecco's Medium. Additionally, the culture medium of the present invention may further include a nutrient mixture. The nutrient mixture refers to a blend that generally includes various amino acids, vitamins, and inorganic salts commonly used in cell culture. This mixture can be prepared by combining the amino acids, vitamins, and inorganic salts, or a commercially available nutrient mixture may be used. Commercially available nutrient mixtures include, for example, F12, M199, MCDB110, MCDB131, MCDB202, and MCDB302, among others. Preferably, F12 nutrient mixture (F12 Nutrient Mixture), M199, or MCDB media may be used.

In the present invention, the type of medium is not particularly limited as long as it is a medium known in the art for culturing mammalian cells, and more preferably, a conventional stem cell culture medium known in the art. For example, the medium may be selected from the group consisting of DMEM medium, M199/F12 medium, MEM-alpha medium, DMEM with low glucose, MCDB131 medium, IMEM medium, K-SFM medium, DMEM/F12 medium, and EGM-2 medium.

In one embodiment of the present invention, the medium used to obtain the conditioned medium may exclude additional serum components, and specifically, may be used in a low-serum medium condition or a serum-free medium condition (a condition considered to have essentially no serum in the art).

In one embodiment of the present invention relating to the method for selecting highly potent stem cells for the treatment of neurological disease, the method may include the following steps:
(I) culturing the stem cells;
(II) measuring the expression levels of TIMP-1, MCP-1, GRO-α, and IL-6 in the stem cells; and
(III) determining and classifying the stem cells as highly potent stem cells (candidates) for the treatment of neurological disease when the expression levels of TIMP-1, MCP-1, GRO-α, and IL-6 are high.

The step (I) is a step of culturing each pool of stem cells, which are in their state prior to selection according to the present invention. If the method is commonly performed in the art, the types of media used (particularly with reference to the description of the basic medium and media mentioned above), culture temperature, culture time, and culture conditions are not particularly limited.

In the step (II), measuring the expression levels of TIMP-1, MCP-1, GROα, and IL-6 specifically refers to measuring the protein expression levels of TIMP-1, MCP-1, GROα, and IL-6, or the expression levels of their respective genes.

The measurement of the expression levels of TIMP-1, MCP-1, GROα, and IL-6 specifically refers to measuring the levels of the proteins or their corresponding genes. The methods for measuring the protein expression levels are not limited in the present invention, as long as they are methods known in the art. For example, methods such as Western blotting, ELISA (enzyme-linked immunosorbent assay), radioimmunoassay, radial immunodiffusion, Ouchterlony immunodiffusion, rocket immunoelectrophoresis, immunohistochemistry, immunoprecipitation, complement fixation assay, flow cytometry, and protein chips, among others, may be used, but are not limited thereto.

The measurement of gene expression levels is not limited to any specific method, as long as it is a technique known in the art. In a specific embodiment, the measurement of the gene expression levels may involve measuring the mRNA expression levels of the genes, but is not limited thereto. The measurement of the mRNA expression levels may be performed using any method known in the art, without limitation. For example, the measurement of mRNA expression levels may be performed using any of the following methods, but is not limited thereto: reverse transcription polymerase chain reaction (RT-PCR), competitive RT-PCR, real-time RT-PCR, quantitative or semi-quantitative RT-PCR, quantitative or semi-quantitative real-time RT-PCR, in situ hybridization, RNase protection assay (RPA), northern blotting, southern blotting, RNA sequencing, DNA chip, or RNA chip.

In step (II), the measurement may be performed on the stem cell culture supernatant cultured in step (I), the conditioned medium obtained after culturing in step (I), or the lysate of the stem cells cultured in step (I), but is not limited thereto.

In the present invention, the term ‘high efficacy’ refers to both the enhanced activity of the stem cells and the significantly superior therapeutic activity against neurological disease, compared to unselected stem cells. Not limited thereto, but preferably, it may refer to significantly superior abilities in neuroprotection, inhibition of reactive gliosis, inhibition of inflammatory response, or angiogenesis in neural tissue.

The step (III) may further include a step of comparing the expression levels of TIMP1, MCP1, GRO-α, and IL-6 measured in step (II) with those of a reference group.

The reference group may preferably refer to the unselected stem cells.

In a specific embodiment of the present invention, step (III) may include comparing the expression levels of TIMP1, MCP1, GRO-α, and IL-6 measured in step (II) with those of a reference group, wherein the reference group is the unselected stem cells. If the levels of TIMP1, MCP1, GRO-α, and IL-6 are increased compared to the reference group, it is determined that the stem cells are ‘overexpressed’ and classified as high-performance stem cells (candidates) for the treatment of neurological disease. In this case, the comparison with the reference group may involve comparing the expression levels of TIMP1, MCP1, GRO-α, and IL-6 with a cutoff value for the expression levels in the reference group, but is not limited thereto. The methods for deriving cutoff values according to various statistical analysis methods are well known in the art.

In one embodiment of the present invention, the cutoff is determined by any one of the methods selected from the group consisting of receiver operating characteristic curve analysis (ROC curve analysis); linear or nonlinear regression methods; linear or nonlinear classification analysis methods: analysis of variance (ANOVA) methods: hierarchical analysis or clustering analysis methods: hierarchical algorithms using decision trees: kernel principal components analysis methods: Markov Blanket analysis methods: recursive feature elimination (RFE) analysis methods; forward or backward floating search (FS) analysis methods; and combinations of two or more of these methods, but is not limited thereto.

In another embodiment of the present invention related to the highly potent stem cell screening method, the method of the present invention may further comprise the following steps: In another embodiment of the method for selecting highly potent stem cells for the treatment of neurological disease of the present invention, the method may further include the step (IV) of measuring whether the stem cells classified in step (III) possess one or more of the following abilities: neuronal differentiation ability; neuroprotective ability: anti-inflammatory ability; and vascular regeneration ability.

In another embodiment of the present invention related to the highly potent stem cell screening method, the method of the present invention may further comprise the following steps: (IV) The step of confirming the preventive or therapeutic effect of the stem cells classified in step (III) on the nervous system disease in a non-human animal model of the nervous system disease.

In a specific embodiment of the method for selecting high-performance stem cells for the treatment of neurological disease according to the present invention, the method may include the following steps:
(i) culturing the stem cells;
(ii) measuring the protein concentrations of TIMP-1, MCP-1, GROα, and IL-6 in the stem cell culture medium from step (i); and
(iii) determining and classifying the stem cells as high-performance stem cells for the treatment of neurological disease when one or more proteins selected from the group consisting of TIMP-1, MCP-1, GROα, and IL-6 are highly expressed in the culture medium.

Step (i) is to be understood with reference to the description of step (I) mentioned earlier. In one embodiment of the present invention, the reference number of stem cells to be cultured in step (i) per cm² is preferably between 8000 and 10000 cells/cm² (i.e., 8000 cells/cm² to 10000 cells/cm²), and more preferably between 8500 and 9500 cells/cm² (i.e., 8500 cells/cm² to 9500 cells/cm²).

In one embodiment of the present invention, the step (i) is preferably performed using one of the media selected from the group consisting of DMEM medium, low-glucose DMEM medium, and DMEM/F12 medium.

In one embodiment of the present invention, the step (i) is preferably performed at a temperature range of 35° C. to 39° C., and more preferably at a temperature range of 36° C. to 38° C.

In one embodiment of the present invention, the step (i) is preferably performed for a culture period of 20 to 30 hours, and more preferably for a culture period of 24 to 26 hours.

In the method, the step (ii) is understood with reference to the description of step (II) mentioned above. In step (ii), the culture medium to be measured is preferably a culture medium that does not contain the stem cells (more preferably, a conditioned medium).

In the method, step (iii) may involve determining that the stem cells are highly potent if the TIMP-1 protein concentration in the culture medium measured in step (ii) is 6,000 μg/mL or higher, the MCP-1 protein concentration is 6,000 μg/mL or higher, the GROα protein concentration is 500 μg/mL or higher, and the IL-6 protein concentration is 50 μg/mL or higher. The present invention is characterized by using these concentrations as cut-off values. A person skilled in the art, with reference to the cut-off feature of the present invention, would be able to derive a range of changes in the cut-off values when altering specific culture conditions. Therefore, such a range of changes (or modified values) is also understood to be included within the scope of the present invention.

In another specific embodiment of the present invention related to the highly potent stem cell selection method, the method of the present invention may further comprise the following steps:
(iv) Measuring whether the stem cells classified in step (iii) possess one or more abilities selected from the group consisting of neuronal differentiation ability, neuroprotective ability, anti-inflammatory ability, and angiogenic ability.

In another specific embodiment of the present invention related to the highly potent stem cell selection method, the method of the present invention may further comprise the following steps:
(iv) Verifying the preventive or therapeutic effect of the stem cells classified in step (iii) in a neurological disease animal (non-human animal) model.

Additionally, the present invention provides a method for determining the neurogenic disease therapeutic activity of stem cells ex vivo, wherein the method comprises measuring and comparing the expression levels of proteins or genes of TIMP-1, MCP-1, GROα, and IL-6 in the stem cells.

In the method for determining the neurogenic disease therapeutic activity of stem cells ex vivo, the proteins or genes of TIMP-1, MCP-1, GROα, and IL-6 are highly expressed in stem cells with high neurogenic disease therapeutic activity, and their composition will be readily understood by those skilled in the art in accordance with the contents described in the aforementioned method for selecting highly potent stem cells for neurogenic disease treatment.

Additionally, the present invention provides a marker composition for selecting highly potent stem cells for neurogenic disease treatment, comprising the proteins or genes of TIMP-1, MCP-1, GROα, and IL-6.

As a specific embodiment, the present invention provides a composition for selecting highly potent stem cells for neurogenic disease treatment, comprising a substance for measuring the expression levels of the proteins or genes of TIMP-1, MCP-1, GROα, and IL-6.

The present invention also provides a kit for selecting highly potent stem cells for neurogenic disease treatment, comprising the composition as described above. In a specific embodiment of the present invention, it provides a kit for selecting highly potent stem cells for neurogenic disease treatment, comprising a substance for measuring the expression levels of the proteins TIMP-1, MCP-1, GROα, and IL-6 or their genes. In another specific embodiment of the present invention, the kit of the present invention may include an instruction manual that describes the method for selecting highly potent stem cells for neurogenic disease treatment, as described above.

Additionally, the present invention provides a formulation for measuring the expression levels of the proteins TIMP-1, MCP-1, GROα, and IL-6, or a composition containing these as active ingredients, for use in selecting highly potent stem cells for neurogenic disease treatment.

Additionally, the present invention provides use for the preparation of a formulation for selecting highly potent stem cells for neurogenic disease treatment, which includes a formulation for measuring the expression levels of the proteins TIMP-1, MCP-1, GROα, and IL-6, or a composition containing these as active ingredients.

Within the composition or kit for selecting highly potent stem cells for neurogenic disease treatment of the present invention, the formulation for measuring the expression levels of the proteins is not specifically limited to a particular type, provided that it is a means known in the art. For example, it may be an antibody specific to the proteins.

Within the composition or kit for selecting highly potent stem cells for neurogenic disease treatment of the present invention, the measurement of the gene expression levels is not specifically limited to a particular type, provided that it is a means and/or method known in the art. For example, it may be measured using a formulation that measures the mRNA expression levels of the genes. The formulation for measuring the mRNA expression levels is not specifically limited to a particular type, provided that it is a means known in the art. For example, it may be a probe or primer set that specifically binds to the mRNA.

The measuring formulations may be labeled with a detectable label to facilitate the identification, detection, measurement, and quantification under the given conditions. For example, the detectable labels may include chemical labels (e.g., biotin), enzyme labels (e.g., horseradish peroxidase, alkaline phosphatase, peroxidase, luciferase, α-glucuronidase, α-galactosidase, chloramphenicol acetyltransferase, β-galactosidase, and β-glucosidase), radioactive labels (e.g., 14C, 125I, 32P, and 35S), fluorescent labels (e.g., coumarin, fluorescein, FITC (fluorescein isothiocyanate), rhodamine 6G, rhodamine B, TAMRA (6-carboxytetramethyl-rhodamine), Cy-3, Cy-5, Texas Red, Alexa Fluor, DAPI (4,6-diamidino-2-phenylindole), HEX, TET, Dabsyl, and FAM), luminescent labels, chemiluminescent labels, FRET (fluorescence resonance energy transfer) labels, or metal labels (e.g., gold and silver). Similarly, the detectable groups may be substrates, cofactors, inhibitors, or affinity ligands.

The confirmation, detection, measurement, and quantification of the measuring substance can be analyzed by detecting the signal emitted from the label. For example, when alkaline phosphatase is used as the label, a colorimetric reaction substrate such as 5-bromo-4-chloro-3-indolyl phosphate (BCIP), nitro blue tetrazolium (NBT), naphthol-AS-B1-phosphate, or enhanced chemifluorescence (ECF) is used to detect the signal. When horseradish peroxidase is used as the label, substrates such as chloronaphthol, aminoethylcarbazole, diaminobenzidine, D-luciferin, lucigenin (bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (tetramethylbenzidine), ABTS (2,2′-Azine-di [3-ethylbenzthiazoline sulfonate]), o-phenylenediamine (OPD), or naphthol/pyrone are used to detect the signal.

Additionally, the kit for selecting highly potent stem cells for neurogenic disease treatment according to the present invention may include, in addition to a formulation for measuring the expression levels of the proteins TIMP-1, MCP-1, GROα, and IL-6, one or more other compositions suitable for the analysis method, the aforementioned labels, substances used for detecting them, and devices.

As a specific embodiment, the kit may be a kit characterized by comprising essential components required to perform reverse transcription polymerase chain reaction. The reverse transcription polymerase chain reaction kit includes a specific pair of primers for each marker gene. The primers are nucleotides with sequences specific to the nucleotide sequences of each marker gene, having a length of about 7 bp to 50 bp, and more preferably about 10 bp to 30 bp. Additionally, primers specific to the nucleotide sequence of a control gene may be included. The reverse transcription polymerase chain reaction kit may also include test tubes or other suitable containers, reaction buffers (with varying pH and magnesium concentrations), deoxynucleotide triphosphates (dNTPs), enzymes such as Taq polymerase and reverse transcriptase, DNase, RNase inhibitors, DEPC-treated water (DEPC-water), sterile water, and the like.

Alternatively, the kit may be characterized by comprising essential components required to perform a DNA chip assay. The DNA chip kit may include a substrate to which cDNA or oligonucleotides corresponding to a gene or its fragment are attached, as well as reagents, formulations, enzymes, and other components for preparing fluorescent-labeled probes. Additionally, the substrate may include cDNA or oligonucleotides corresponding to a control gene or its fragment.

Alternatively, the kit may be characterized by including essential components required to perform an ELISA. The ELISA kit includes antibodies specific to the marker proteins. The antibodies are those with high specificity and affinity for each marker protein and minimal cross-reactivity with other proteins, and may include monoclonal antibodies, polyclonal antibodies, or recombinant antibodies. Additionally, the ELISA kit may include antibodies specific to the control proteins. Additionally, the ELISA kit may include reagents capable of detecting the bound antibodies, such as labeled secondary antibodies, chromophores, enzymes (e.g., conjugated with the antibodies), substrates for the enzymes, or other substances that can bind to the antibodies.

Additionally, the kit of the present invention may include a washing solution or elution buffer that removes unbound proteins, such as enzymes and substrates for colorimetric reactions, and retains only the bound protein markers.

Additionally, the present invention provides a screening method for a neurogenic disease prevention or treatment agent, comprising the steps of:
(a) contacting a test substance with isolated stem cells;
(b) measuring the expression levels of proteins or their genes of TIMP-1, MCP-1, GROα, and IL-6 in the stem cells contacted with the test substance and in control stem cells not contacted with the test substance; and
(c) selecting a test substance in which the expression or activity levels of TIMP-1, MCP-1, GROα, and IL-6 proteins or their genes are increased, compared to the control cells.

In the screening method of the present invention, the measurement of protein expression or activity levels may be performed using any method known in the art, without limitation. For example, the measurement may be performed using any one or more methods selected from the group consisting of Western blot, ELISA (enzyme-linked immunosorbent assay), radioimmunoassay, radial immunodiffusion, Ouchterlony immunodiffusion, rocket immunoelectrophoresis, immunohistochemical staining, immunoprecipitation analysis, complement fixation assay, flow cytometry (FACS), and protein chips, but is not limited thereto.

In the screening method of the present invention, the measurement of gene expression or activity levels may be performed using any method known in the art, without limitation. In a specific embodiment, the measurement of gene expression or activity levels may involve measuring the mRNA expression or activity levels for each gene, but is not limited thereto. In this case, the measurement of mRNA expression or activity levels may be carried out using any method known in the art, without limitation. For example, the measurement of mRNA expression or activity levels may be carried out using one or more methods selected from the group consisting of reverse transcription polymerase chain reaction (RT-PCR), competitive RT-PCR, real-time RT-PCR, quantitative or semi-quantitative RT-PCR, quantitative or semi-quantitative real-time RT-PCR, in situ hybridization, RNase protection assay (RPA), northern blotting, southern blotting, RNA sequencing, DNA chip, and RNA chip, but is not limited thereto.

The screening method comprising steps (a), (b), and (c) of the present invention can be understood by those skilled in the art as a method for screening candidate substances for the prevention and/or treatment of neurogenic diseases.

Thus, in one embodiment of the present invention, the present invention provides a screening method for candidate substances for the prevention and/or treatment of neurogenic diseases, comprising the steps of:
(a) contacting a separated stem cell with a test substance;
(b) measuring the expression levels of the proteins TIMP-1, MCP-1, GROα, and IL-6, or the expression levels of their genes, in stem cells contacted with the test substance and in control stem cells not contacted with the test substance; and
(c) identifying and/or selecting a test substance as a candidate substance, where the expression or activity levels of the proteins TIMP-1, MCP-1, GROα, and IL-6, or their genes, are increased compared to control cells.

The screening method comprising steps (a), (b), and (c) of the present invention can be understood by those skilled in the art as a method for screening stem cell adjuncts (stem cell activators, stem cell activation-promoting combination agents, or neurogenic disease prevention or treatment stem cell activators) for the prevention or treatment of neurogenic diseases.

Therefore, in one embodiment of the present invention, the present invention provides a screening method for stem cell adjuncts (stem cell activators, neurogenic disease prevention or treatment stem cell activation-promoting agents (activators)) for the prevention or treatment of neurogenic diseases, comprising the steps of:
(a) contacting a test substance with isolated stem cells;
(b) measuring the expression or activity levels of the proteins TIMP-1, MCP-1, GROα, and IL-6, or their genes, in stem cells contacted with the test substance and control stem cells not contacted with the test substance; and
(c) screening the test substance based on the increased expression or activity levels of TIMP-1, MCP-1, GROα, and IL-6 proteins or their genes, compared to the control cells.

In the aforementioned screening methods of the present invention, the measurement of the expression levels of TIMP-1, MCP-1, GROα, and IL-6 proteins or their genes in step (b) may involve measuring the expression (secretion) levels in the culture medium in which the stem cells contacted with the test substance and the control stem cells not contacted with the test substance are cultured.

In the aforementioned screening methods of the present invention, the term ‘selection’ in step (c) may refer to the selection of the test substance based on the steps or selection criteria (for example, cutoff values, etc.) of the ‘highly potent stem cell selection method for neurogenic disease treatment’ as described in the present invention.

Specifically, in one embodiment of the present invention, the ‘selection’ in step (c) is preferably performed by selecting the test substance based on the following criteria: culturing stem cells at a density of 8000 cells/cm² to 10000 cells/cm² (more preferably 8500 cells/cm² to 9500 cells/cm²), and selecting the test substance in which the concentration of TIMP-1 protein secreted from the stem cells is increased to 6,000 μg/mL or more, the concentration of MCP-1 protein is 6,000 μg/mL or more, the concentration of GROα protein is 500 μg/mL or more, and the concentration of IL-6 protein is 50 μg/mL or more. The present invention may be characterized by using the above concentrations as cut-off values. A person skilled in the art would readily understand that, by referring to the cut-off characteristics of the present invention, the range of changes in the cut-off value can be derived by altering specific culture conditions. Therefore, such a range of changes (modified values) is also included within the scope of the present invention.

The aforementioned screening methods of the present invention may further include the following step after step (c), which allows for the final selection of the test substance:
(d) A step of confirming the neurogenic disease prevention or treatment effect of the test substance (candidate for prevention or treatment) selected in step (c) in a neurogenic disease animal (non-human animal) model.

In step (d), the (candidate) test substance may be co-administered (combinatorially administered) with the stem cells from step (a), but is not limited thereto.

Additionally, the present invention provides a composition for promoting the activity of stem cells for the prevention or treatment of neurogenic diseases, which includes as an active ingredient a substance that increases the expression or activity of the genes or proteins TIMP-1, MCP-1, GROα, and IL-6.

Additionally, the present invention provides a method for promoting the activity of stem cells for the prevention or treatment of neurogenic diseases, which includes the step of treating isolated stem cells with a substance that increases the expression or activity of the genes or proteins TIMP-1, MCP-1, GROα, and IL-6.

Additionally, the present invention provides the use of a substance that increases the expression or activity of the genes or proteins TIMP-1, MCP-1, GROα, and IL-6; or a composition containing these as active ingredients, for promoting the activity of stem cells for the prevention or treatment of neurogenic diseases.

Additionally, the present invention provides the use of a substance that increases the expression or activity of the genes or proteins TIMP-1, MCP-1, GROα, and IL-6; or a composition containing these as active ingredients, for the preparation of a formulation for promoting the activity of stem cells for the prevention or treatment of neurogenic diseases.

The activity of the stem cells is not limited to, but preferably may include one or more selected from the group consisting of self-renewal (neurogenesis), neuroprotective ability, inhibition of neurocyte apoptosis, endothelial cell migration, angiogenesis, anti-inflammatory effects, and inhibition of reactive gliosis.

In this specification, the term ‘increase material’ encompasses the meanings of terms commonly used in the art such as enhancer, promoter, booster, and activator.

In this specification, the term ‘protein expression or activity increase’ includes the meaning of an increase in the extracellular secretion of the relevant protein.

The protein expression or activity increasing substance is not limited to the following, but an exemplary preferred embodiment may include one or more expression vectors (recombinant expression vectors) containing Polynucleotides encoding the TIMP-1 protein: polynucleotides encoding the MCP-1 protein; polynucleotides encoding the GROα protein; and polynucleotides encoding the IL-6 protein.

As a specific embodiment of the present invention, the protein expression or activity increasing substance provides one or more expression vectors (recombinant expression vectors) comprising a promoter and one or more polynucleotides operably linked thereto, as follows:

Polynucleotides encoding the TIMP-1 protein: polynucleotides encoding the MCP-1 protein: polynucleotides encoding the GROα protein; and polynucleotides encoding the IL-6 protein.

The polynucleotides encoding each of the TIMP-1, MCP-1, GROα, or IL-6 proteins provided in the present invention may be incorporated in a single expression vector, or they may be incorporated individually or in combinations of two or more expression vectors, for example, in 2 to 4 expression vectors.

The term ‘promoter’ refers to a DNA sequence that regulates the expression of a polynucleotide sequence operably linked to it in a particular host cell, and “operably linked” means that one polynucleotide fragment is connected to another polynucleotide fragment in such a way that its function or expression is influenced by the other polynucleotide fragment. Additionally, the sequence may further include any operator sequences for regulating transcription, sequences coding for suitable mRNA ribosome binding sites, and sequences for regulating transcription and translation termination. The promoter may be a constitutive promoter that induces expression of the target gene at all times or an inducible promoter that induces expression of the target gene at specific locations or times. Examples of such promoters include the SV40 promoter, CMV promoter, CAG promoter, CaMV 35S promoter, Rsyn7 promoter (U.S. patent application Ser. No. 08/991,601), rice actin promoter, ubiquitin promoter, and ALS promoter (U.S. patent application Ser. No. 08/409,297), among others. Additionally, promoters disclosed in U.S. Pat. Nos. 5,608,149; 5,608,144; 5,604,121; 5,569,597; 5,466,785; 5,399,680; 5,268,463; and 5,608,142, among others, can also be used.

Meanwhile, the expression vector can be introduced into target cells (host cells) in an expression form by methods known in the art, such as infection, transfection, or transduction.

The gene delivery method using a plasmid expression vector is a method of directly delivering plasmid DNA to mammalian cells, and it is a method approved by the FDA for use in humans. Plasmid DNA has the advantage of being able to be uniformly purified, unlike viral vectors. The plasmid expression vectors that can be used in the present invention include mammalian expression plasmids known to those skilled in the art. For example, but not limited to, pRK5 (European Patent No. 307,247), pSV16B (International Patent Publication No. 91/08291), and pVL1392 (PharMingen) are representative. The plasmid expression vectors can be introduced into target cells by methods known in the art, including, but not limited to, transient transfection, microinjection, transduction, cell fusion, calcium phosphate precipitation, liposome-mediated transfection, DEAE dextran-mediated transfection, polybrene-mediated transfection, electroporation, gene gun, and other known methods for introducing DNA into cells.

Additionally, as methods applicable to the present invention, viral expression vectors containing the nucleic acid include, but are not limited to, retroviruses, adenoviruses, herpes viruses, avipox viruses, and lentiviruses. The retrovirus vector is one in which all viral genes have been removed or altered so that non-viral proteins are produced within the cells infected by the viral vector. The main advantages of retrovirus vectors for gene therapy are their ability to deliver large amounts of genes into replicating cells, to accurately integrate the delivered genes into the cell's DNA, and to prevent continuous infection after gene transfection. The retrovirus vector approved by the FDA is produced using the PA317 amphotropic retrovirus packaging cell line. Non-retrovirus vectors include adenoviruses, as mentioned above. The main advantage of adenoviruses is their ability to carry large DNA fragments (36 kb genome) and infect non-replicating cells with very high titers. Additionally, herpes viruses can also be usefully employed for human gene therapy. In addition, other known suitable viral vectors may be used in the present invention.

Additionally, the present invention provides a pharmaceutical composition for the prevention or treatment of neurological disease, comprising stem cells (stem cell therapy) that overexpress the genes or proteins of TIMP-1, MCP-1, GROα, and IL-6, and/or a culture medium thereof as an active ingredient.

Additionally, the present invention provides a method for the prevention, improvement, and/or treatment of neurological disease, comprising the step of administering to a subject in need thereof stem cells overexpressing the genes or proteins of TIMP-1, MCP-1, GROα, and IL-6, or their culture medium; or a composition comprising these as active ingredients.

Additionally, the present invention provides the use of stem cells overexpressing the genes or proteins of TIMP-1, MCP-1, GROα, and IL-6, or their culture medium; or a composition comprising these as active ingredients, for the prevention, improvement, and/or treatment of neurological disease.

Additionally, the present invention provides the use of stem cells overexpressing the genes or proteins of TIMP-1, MCP-1, GROα, and IL-6, or their culture medium; or a composition comprising these as active ingredients, for the manufacture of a preparation (product) for the prevention, improvement, and/or treatment of neurological disease.

Additionally, the present invention provides a pharmaceutical composition for inducing angiogenesis, comprising stem cells overexpressing the genes or proteins of TIMP-1, MCP-1, GROα, and IL-6 (stem cell therapeutic agents), and/or their culture medium as active ingredients.

The culture medium may be a conditioned medium containing the proteins of TIMP-1, MCP-1, GROα, and IL-6 secreted by the stem cells, but is not limited thereto. Additionally, the present invention provides a method for inducing angiogenesis, comprising the step of administering to a subject in need thereof stem cells overexpressing the genes of TIMP-1, MCP-1, GROα, and IL-6 or their proteins, or the culture thereof, or a composition comprising these as active ingredients.

Additionally, the present invention provides a method for the prevention, improvement, and/or treatment of cerebrovascular disease, comprising the step of administering to a subject in need thereof stem cells overexpressing the genes of TIMP-1, MCP-1, GROα, and IL-6 or their proteins, or the culture thereof, or a composition comprising these as active ingredients.

Additionally, the present invention provides the use of stem cells overexpressing the genes of TIMP-1, MCP-1, GROα, and IL-6 or their proteins, or the culture thereof, or a composition comprising these as active ingredients, for inducing angiogenesis.

Additionally, the present invention provides the use of stem cells overexpressing the genes of TIMP-1, MCP-1, GROα, and IL-6 or their proteins, or the culture thereof, or a composition comprising these as active ingredients, for the prevention, improvement, and/or treatment of cerebrovascular disease.

Additionally, the present invention provides the use of stem cells overexpressing the genes of TIMP-1, MCP-1, GROα, and IL-6 or their proteins, or the culture thereof, or a composition comprising these as active ingredients, for the preparation of a therapeutic agent for inducing angiogenesis.

Additionally, the present invention provides the use of stem cells overexpressing the genes of TIMP-1, MCP-1, GROα, and IL-6 or their proteins, or the culture thereof, or a composition comprising these as active ingredients, for the preparation (production) of a therapeutic agent for the prevention, improvement, and/or treatment of cerebrovascular disease.

In this specification, the term ‘stem cell therapeutic agent’ is used in its commonly accepted meaning in the art, and preferably refers to a pharmaceutical composition containing stem cells as an active ingredient. The term ‘administration’ of stem cells in this specification includes the meaning of ‘implantation’ of the cells.

In the present invention, the “overexpression of the genes of TIMP-1, MCP-1, GROα, and IL-6 or their proteins” can be achieved by treatment (or contact) with a substance that increases the expression or activity of the genes or proteins of TIMP-1, MCP-1, GROα, and IL-6 as described above.

In a preferred embodiment of the present invention, the overexpression refers to a process in which a gene containing a sequence that encodes the respective polypeptides of TIMP-1, MCP-1, GROα, or IL-6 is artificially expressed within a cell to generate a level of expressed encoded polypeptides that exceeds the expression level of the same polypeptide in a precursor host cell. Therefore, when the above term is used in conjunction with a gene, the term “overexpression” may also be used together with the protein to refer to the increased level of the protein generated from the overexpression of the gene that encodes it. In some embodiments, the overexpression of the gene encoding the protein is achieved by increasing the number of copies of the gene encoding the protein. In other embodiments, the overexpression of the gene encoding the protein is achieved by increasing the binding affinity of the promoter region and/or ribosome binding site to enhance the transcription and/or translation of the gene encoding the protein. In other embodiments, the overexpression can be achieved by increasing the copy number of the gene and enhancing the binding affinity of the promoter region and/or ribosome binding site. In some embodiments, the overexpression of a gene encoding a protein results from the expression of one or more copies of the corresponding encoding polynucleotide present in multiple copies of plasmids introduced into the genome of the host cell. In other embodiments, the overexpression of a gene encoding a protein results from the expression of two or more copies of the corresponding encoding polynucleotide integrated into the genome of the host cell.

Additionally, the present invention provides a pharmaceutical composition for the prevention or treatment of neurological disease, comprising isolated stem cells and a substance that increases the expression or activity of TIMP-1, MCP-1, GROα, and IL-6 genes or their proteins as the active ingredient, as well as a pharmaceutical combination preparation for the prevention or treatment of neurological disease comprising the composition.

Additionally, the present invention provides a method for the prevention, improvement, and/or treatment of neurological disease, comprising the step of administering to a subject in need thereof isolated stem cells and a substance that increases the expression or activity of TIMP-1, MCP-1, GROα, and IL-6 genes or their proteins, or a composition comprising the substance as the active ingredient.

Additionally, the present invention provides the use of isolated stem cells and a substance that increases the expression or activity of TIMP-1, MCP-1, GROα, and IL-6 genes or their proteins, or a composition comprising the substance as the active ingredient, for the prevention, improvement, and/or treatment of neurological disease.

Additionally, the present invention provides the use of isolated stem cells and a substance that increases the expression or activity of TIMP-1, MCP-1, GROα, and IL-6 genes or their proteins, or a composition comprising the substance as the active ingredient, for the manufacture (production) of a preparation (especially a combination or co-therapy formulation) for the prevention, improvement, and/or treatment of neurological disease.

Additionally, the present invention provides an angiogenesis-inducing pharmaceutical composition comprising isolated stem cells and a substance that increases the expression or activity of TIMP-1, MCP-1, GROα, and IL-6 genes or their proteins as the active ingredient, as well as an angiogenesis-inducing pharmaceutical composition comprising the same.

Additionally, the present invention provides an angiogenesis-inducing method comprising the step of administering to a subject in need thereof isolated stem cells and a substance that increases the expression or activity of TIMP-1, MCP-1, GROα, and IL-6 genes or their proteins as the active ingredient, or a composition containing the same, particularly by co-administration or combination administration.

Additionally, the present invention provides a method for the prevention, improvement, and/or treatment of cerebrovascular disease, comprising the step of administering to a subject in need thereof (isolated stem cells) and a substance that increases the expression or activity of TIMP-1, MCP-1, GROα, and IL-6 genes or their proteins as the active ingredient, or a composition containing the same, particularly by (co-administration) or (combination administration).

Additionally, the present invention provides the use of isolated stem cells and a substance that increases the expression or activity of TIMP-1, MCP-1, GROα, and IL-6 genes or their proteins: or a composition containing the same as an active ingredient for inducing angiogenesis.

Additionally, the present invention provides the use of isolated stem cells and a substance that increases the expression or activity of TIMP-1, MCP-1, GROα, and IL-6 genes or their proteins: or a composition containing the same as an active ingredient for the prevention, improvement, and/or treatment of cerebrovascular disease.

Additionally, the present invention provides the use of isolated stem cells and a substance that increases the expression or activity of TIMP-1, MCP-1, GROα, and IL-6 genes or their proteins: or a composition containing the same as an active ingredient for the preparation (production) of a vascular formation-inducing formulation (particularly, a combination formulation or a co-administered formulation).

Additionally, the present invention provides the use of isolated stem cells and a substance that increases the expression or activity of TIMP-1, MCP-1, GROα, and IL-6 genes or their proteins: or a composition containing the same as an active ingredient for the preparation (production) of a formulation for the prevention, improvement, and/or treatment of cerebrovascular disease (particularly, a combination formulation or a co-administered formulation).

In the present invention, the term ‘for inducing angiogenesis’ may preferably refer to the use intended for the prevention or treatment of cerebrovascular disease. The cerebrovascular disease described above refer to neurologic deficits caused by impaired normal blood supply to the brain. The types of cerebrovascular disease are not particularly limited, as long as they are recognized in the art. Examples include ischemic stroke, cerebral ischemia, cerebrovascular accidents, vascular dementia, intracerebral hemorrhage, subarachnoid hemorrhage, and leukodystrophy, any one or more of which may be selected from the group.

The pharmaceutical composition according to the present invention may be formulated in the form of a single composition, where the isolated stem cells and the gene or activation agents of TIMP-1, MCP-1, GROα, and IL-6 are combined, or may be formulated in the form of separate compositions. The method of formulating these components can utilize commonly known techniques in the art.

The genes of TIMP-1, MCP-1, GROα, and IL-6, or their expression or activity-enhancing substances, may be administered simultaneously with the isolated stem cells, individually, or sequentially.

The active ingredients of the present invention, including the isolated stem cells and the genes of TIMP-1, MCP-1, GROα, and IL-6, or their expression or activity-enhancing substances, may be included together in a single pharmaceutical preparation and administered simultaneously through the same administration site; or provided as individual preparations and administered either simultaneously or sequentially through the same administration site; or provided as individual preparations and administered either simultaneously or sequentially through different administration sites.

Specifically, the term ‘simultaneously’ refers to administering the two active ingredients together through the same administration route, or administering them via the same or different administration routes at substantially the same time (e.g., within an administration time interval of 15 minutes or less). The term ‘separately’ refers to administering the two active ingredients with a certain time interval through the same or different administration routes. The term ‘sequentially’ refers to administering the two active ingredients in a specific order, depending on the patient's disease condition, through the same or different administration routes. The administration sequence of the pharmaceutical composition according to the present invention, including which component to administer at what time, whether simultaneously, separately, or sequentially, can be determined by a physician or expert. Such an administration sequence may vary depending on numerous factors.

The administration route may be oral or parenteral. It may be administered through various routes. All methods of administration may be anticipated by those skilled in the art and include, but are not limited to, oral administration, subcutaneous injection, intraperitoneal administration, intravenous injection, intramuscular injection, epidural injection, sublingual administration, buccal administration, rectal insertion, vaginal insertion, ocular administration, otic administration, nasal administration, inhalation, spraying through the mouth or nose, dermal administration, and transdermal administration.

The pharmaceutical composition according to the present invention may include only the above active ingredients in a pharmaceutically effective amount or may additionally include suitable carriers, excipients, and diluents commonly used in the preparation of pharmaceutical compositions. The term ‘pharmaceutically effective amount’ refers to an amount that produces a response greater than that of a negative control, and preferably refers to an amount sufficient to treat or prevent a neurologic disorder.

The combination ratio, dosage, and timing of administration of the active ingredients of the present invention, which are selected from a group consisting of isolated stem cells and one or more of the genes or their protein expression or activity-enhancing agents, can be selected and administered by a person skilled in the art within the scope of the present invention, taking into account factors such as the patient's level of pain, sensitivity to the drug, side effects, and other considerations.

On the other hand, when the active ingredients of the present invention are co-administered as a single formulation, the composition (formulation) can be prepared by appropriately selecting the ratio of each component based on the amount of each ingredient in the daily dose. This combined formulation can then be administered once or multiple times a day.

However, the pharmaceutically effective amount may vary appropriately depending on several factors such as the disease and its severity, the patient's age, weight, health condition, gender, administration route, and treatment duration. For example, when administered to human patients, the total daily dose of the composition according to the present invention may be determined by medical judgment within the scope of treatment. The specific therapeutic effective amount for a particular patient should be adjusted based on various factors, including the type and extent of the response desired, whether different formulations are used in certain cases, the patient's age, weight, general health status, gender, diet, dosing time, administration route, secretion rate of the composition, treatment duration, and other similar factors well known in the pharmaceutical field.

The pharmaceutical composition according to the present invention may further include appropriate carriers, excipients, and diluents commonly used in the preparation of pharmaceutical compositions. The carriers include all types of solvents, dispersing media, water-in-oil or oil-in-water emulsions, aqueous compositions, liposomes, microbeads, and microsponges.

The excipients may include one or more selected from the group consisting of diluents, binders, disintegrants, lubricants, adsorbents, humectants, film-coating materials, and controlled-release additives.

The pharmaceutical composition according to the present invention can be formulated in various forms, such as powders, granules, sustained-release granules, enteric-coated granules, liquids, eye drops, elixirs, emulsions, suspensions, tinctures, syrups, lozenges, aerosols, tablets, sustained-release tablets, enteric-coated tablets, sublingual tablets, hard capsules, soft capsules, sustained-release capsules, enteric-coated capsules, powders, tinctures, soft extracts, dry extracts, fluid extracts, injectables, capsules, perfusates, lotions, pastes, sprays, inhalants, patches, sterile injectable solutions, or aerosols. The topical preparations may have forms such as creams, gels, patches, sprays, ointments, lotions, liniments, pastes, or cataplasms.

The carriers, excipients, and diluents that can be included in the pharmaceutical composition according to the present invention include lactose, dextrose, sucrose, oligosaccharides, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methylhydroxybenzoate, propylhydroxybenzoate, talc, magnesium stearate, and mineral oil.

The composition according to the present invention can also be formulated as a non-oral administration preparation. Preparations for non-oral administration include sterilized aqueous solutions, non-aqueous solvents, suspensions, emulsions, freeze-dried preparations, and suppositories. For sterilized aqueous solutions, suitable buffered solutions such as Hank's solution, Ringer's solution, or physiologically buffered saline can be used. Non-aqueous solvents and suspensions can include propylene glycol, polyethylene glycol, vegetable oils such as olive oil, and injectable esters such as ethyl oleate. If necessary, preservatives, stabilizers, wetting agents or emulsifiers, salts and/or buffers for osmolality adjustment can be used. For suppositories, conventional bases such as Witepsol, Macrogol, Tween 61, cacao butter, lauric acid, and glycerogelatin can be used.

When formulating, the composition is typically prepared using diluents or excipients such as commonly used fillers, bulking agents, binders, wetting agents, disintegrants, and surfactants.

The additives for tablets, powders, granules, capsules, lozenges, and troches according to the present invention may include corn starch, potato starch, wheat starch, lactose, sucrose, glucose, fructose, D-mannitol, precipitated calcium carbonate, synthetic aluminum silicate, monocalcium phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropylmethylcellulose (HPMC) 1928, HPMC 2208, HPMC 2906, HPMC 2910, propylene glycol, casein, calcium lactate, and primogel as excipients: gelatin, arabic gum, ethanol, agar powder, cellulose acetate phthalate, carboxymethylcellulose, carboxymethylcellulose calcium, glucose, purified water, sodium caseinate, glycerin, stearic acid, sodium carboxymethylcellulose, methylcellulose sodium, methylcellulose, microcrystalline cellulose, dextrin, hydroxycellulose, hydroxypropyl starch, hydroxypropylmethylcellulose, purified shellac, starch powder, hydroxypropylcellulose, hydroxypropylmethylcellulose, polyvinyl alcohol, polyvinylpyrrolidone as binders: hydroxypropylmethylcellulose, corn starch, agar powder, methylcellulose, bentonite, hydroxypropyl starch, sodium carboxymethylcellulose, sodium alginate, carboxymethylcellulose calcium, calcium citrate, sodium lauryl sulfate, anhydrous silica, 1-hydroxypropylcellulose, dextran, ion-exchange resins, polyvinyl acetate, formaldehyde-treated casein and gelatin, alginate, amylose, guar gum, sodium bicarbonate, polyvinylpyrrolidone, calcium phosphate, gelling starch, arabic gum, amylopectin, pectin, polyphosphate sodium, ethylcellulose, sucrose, magnesium silicate, D-sorbitol solution, and anhydrous silica as disintegrants; calcium stearate, magnesium stearate, stearic acid, hydrogenated vegetable oil, talc, kaolin, vaseline, sodium stearate, cocoa butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, macrogol, synthetic aluminum silicate, anhydrous silica, high fatty acids, high molecular alcohols, silicone oil, paraffin oil, polyethylene glycol fatty acid esters, starch, sodium chloride, sodium acetate, oleic acid sodium, dl-leucine, and anhydrous silica as lubricants.

Additives for liquid formulations according to the present invention may include water, dilute hydrochloric acid, dilute sulfuric acid, sodium citrate, sucrose monoester of stearic acid, polyoxyethylene sorbitan fatty acid esters (Tween esters), polyoxyethylene monoalkyl ethers, lanolin ethers, lanolin esters, acetic acid, hydrochloric acid, ammonia solution, ammonium carbonate, potassium hydroxide, sodium hydroxide, prolamine, polyvinylpyrrolidone, ethylcellulose, sodium carboxymethylcellulose, and the like.

Syrup formulations according to the present invention may include a solution of sucrose, other sugars, or sweeteners, and, if necessary, may also include flavoring agents, colorants, preservatives, stabilizers, suspending agents, emulsifiers, and thickeners.

Emulsions according to the present invention may include purified water, and, if necessary, emulsifiers, preservatives, stabilizers, and flavoring agents may be used.

Suspensions according to the present invention may include suspending agents such as acacia, tragacanth, methylcellulose, carboxymethylcellulose, carboxymethylcellulose sodium, microcrystalline cellulose, sodium alginate, hydroxypropylmethylcellulose, HPMC 1828, HPMC 2906, HPMC 2910, and the like, and, if necessary, surfactants, preservatives, stabilizers, coloring agents, and flavoring agents may be used.

Injectables according to the present invention may include solvents such as sterile water for injection, 0.9% sodium chloride injection, Ringer's injection, dextrose injection, dextrose+sodium chloride injection, PEG (polyethylene glycol), lactated Ringer's injection, ethanol, propylene glycol, non-volatile oils such as perilla oil, cottonseed oil, safflower oil, soybean oil, corn oil, oleic acid ethyl ester, isopropyl myristate, and benzyl benzoate; solubilizing agents such as sodium benzoate, sodium salicylate, sodium acetate, urea, urethane, monoethyl acetamide, butazolidine, propylene glycol, Tween types, niacinamide, hexamine, dimethyl acetamide; buffers such as weak acids and their salts (acetic acid and sodium acetate), weak bases and their salts (ammonia and ammonium acetate), organic compounds, proteins, albumin, peptone, and gums; denaturants such as sodium chloride; stabilizers such as sodium bisulfite (NaHSO3), carbon dioxide gas, sodium metabisulfite (Na2S2O5), sodium sulfite (Na2SO3), nitrogen gas (N2), and ethylenediaminetetraacetic acid; antioxidants such as sodium bisulfite 0.1%, sodium formaldehyde sulfoxylate, thiourea, disodium ethylenediaminetetraacetic acid, and sodium bisulfite acetone; anesthetics such as benzyl alcohol, chlorobutanol, procaine hydrochloride, glucose, and calcium gluconate; suspending agents such as sodium CMC, sodium alginate, Tween 80, and monostearin aluminum may be included.

Suppositories according to the present invention may include excipients such as cocoa butter, lanolin, Witepsol, polyethylene glycol, glycerogelatin, methylcellulose, carboxymethylcellulose, a mixture of stearic acid and oleic acid, Subanal, cottonseed oil, safflower oil, palm oil, cocoa butter+cholesterol, lecithin, lanette wax, monostearin glycerol, Tween or Span, Imhausen, Monolen (monostearic acid propylene glycol), glycerin, Adeps solidus, Buytyrum Tego-G, Cebes Pharma 16, Hexaride Base 95, Cotomar, Hydrocote SP, S-70-XXA, S-70-XX75 (S-70-XX95), Hydrocote 25, Hydrocote 711, Idropostal, Massa estrarium (A, AS, B, C, D, E, I, T), Massa-MF, Masupol, Masupol-15, Neosufostal-N, Paramount-B, Suposhiro (OSI, OSIX, A, B, C, D, H, L), Suppository Base IV Type (AB, B, A, BC, BBG, E, BGF, C, D, 299), Supostal (N, Es), Wecobi (W, R, S, M, Fs), and Tejester triglyceride base (TG-95, MA, 57).

The pharmaceutical composition according to the present invention is not specifically limited in terms of its formulation, administration route, or administration method, as long as it exhibits the effects of the present invention.

The pharmaceutical composition according to the present invention is administered in a pharmaceutically effective amount. In the present invention, the term “pharmaceutically effective amount” refers to an amount sufficient to treat the disease with a reasonable benefit/risk ratio applicable to medical treatment, and the effective dosage level can be determined based on factors such as the type and severity of the patient's disease, the activity of the drug, the sensitivity to the drug, administration time, administration route, excretion rate, treatment duration, and other factors well known in the medical field, including any concurrently used medications.

The pharmaceutically combined formulation of the present invention may be formulated such that the active ingredients, as components, are included simultaneously in a single dosage form according to the method of administration and the administration route. Alternatively, each component may be individually formulated and included in a single package according to the dosage unit, such as daily or single-dose administration. The formulations of the active ingredients, which are individually formulated, may either be the same or different. The specific types, formulation methods, and pharmaceutically acceptable carriers that may be included in the pharmaceutical combination preparation of the present invention are as described above in the pharmaceutical composition.

In the present invention, the term ‘pharmaceutically acceptable’ refers to non-toxic substances or compositions that are physiologically acceptable and do not inhibit the action of the active ingredient when administered to humans, and that typically do not cause allergic reactions or similar reactions such as gastrointestinal disturbances or dizziness.

The active ingredients as components of the pharmaceutical combination formulation according to the present invention can be administered simultaneously, individually, or in a predetermined order (sequentially). The combination formulation may be formulated so that the daily dose is included in a single administration, but it can also be formulated to be administered in divided doses, such as 2, 3, or 4 times per day.

The preferred dose of the pharmaceutical combination formulation of the present invention may vary appropriately depending on several factors, such as the disease and its severity, the patient's age, weight, health condition, gender, route of administration, and treatment period. Since the biological availability (bioavailability) of the pharmaceutical active ingredient can vary between individuals, it may be desirable to monitor the blood concentration of each drug using assays based on single-clone monoclonal antibodies or the like at the initial stage of administration of the pharmaceutical formulation of the present invention, as known in the art.

In the present invention, the term ‘prevention’ refers to any act of inhibiting or delaying the onset of the disease targeted by the present invention (in this invention, neurological disease) through the administration of the pharmaceutical composition of the present invention before the onset of the targeted disease.

In the present invention, the term “treatment” refers to any act in which the symptoms of the disease targeted by the present invention (in this invention, neurological disease) are improved or beneficially altered through the administration of the pharmaceutical composition of the present invention after the onset of the targeted disease. In the present invention, the term ‘improvement’ refers to any act that reduces parameters related to the disease targeted by the present invention, such as the degree of symptoms.

In the present invention, the term ‘subject’ refers to an entity that requires treatment for a disease (not limited thereto, but in this invention, it may refer to a subject that needs inhibition of neuronal cell damage and induction of angiogenesis within the brain as an example). More specifically, it can be a mammal, including humans or non-human mammals such as mice, rats, dogs, cats, horses, and cattle, but is not limited to thereto.

Below, preferred embodiments and experimental examples are provided to assist in understanding the present invention. However, the following embodiments and experimental examples are provided solely for the purpose of facilitating a better understanding of the present invention, and the scope of the present invention is not limited to the contents of the following embodiments and experimental examples.

### Example 1. Obtaining Conditioned Media of Selectively Isolated Neural Stem Cells According to the Present Invention, Cytokine Analysis, and Liquid Chromatography-Mass Spectrometry (LC/MS/MS) Analysis

### 1-1. Culturing of Neural Stem Cells and Obtaining Conditioned Media.

Neural stem cells were isolated and cultured from adult human brain tissue (temporal lobe tissue from a donor with no lesions, classified as type IIIa focal cortical dysplasia). First, the surgical specimens were mechanically minced, followed by treatment with an enzyme mixture containing papain (10 units/ml, Sigma), DNase I (0.1 mg/ml, Roche), and D,L-cysteine (4 mg/ml, Sigma) at 37° C. for 15 to 30 minutes. Subsequently, after a gentle grinding process, the mixture was filtered using a cell strainer (40 μm, BD Biosciences). After Percoll purification, the cells were cultured in an adherent culture on poly-L-ornithine (PLO)-coated dishes using DMEM/F12 medium supplemented with 1% B27 (Invitrogen), 1% penicillin/streptomycin cocktail (Invitrogen), EGF (50 ng/ml, R & D Systems), bFGF (50 ng/ml, R & D Systems), and 0.5% FBS (Invitrogen). Subsequently, the cells were passaged using Accutase (PAA Laboratories, Austria) and cultured to 80% confluence. Using the above method, various human neural stem cell lines were obtained to create different pools of neural stem cell lines.

After thawing the cryopreserved neural stem cells, 6,000 cells per cm² were seeded into a T75 flask containing 20 mL of the proliferation medium, which includes 0.1 mL of FBS, 0.4 mL of B-27™ supplement (Xenofree; Gibco, A14867-01), 0.2 mL of N−2 supplement (Gibco, 17502-048), 1 μL of hEGF, 1 μL of hbFGF2, and 10 μL of Gentamicin in DMEM/F12 (Gibco, 11330-032) medium. The neural stem cells were cultured in the T75 flask until reaching 80% to 90% confluence, and then washed three times with PBS. Subsequently, the cells were detached using trypsin/EDTA, the cell count was measured, and then 9,000 cells per cm² were seeded into a T175 flask containing proliferation medium for subculture (CO2 incubator [Thermofisher (HERACELL VIOS 160i)], 37.0° C., 5% CO2). Forty-eight hours after the cell subculture, the obtained culture medium was replaced with a basal medium DMEM/F12 [Gibco (11330-032)] containing Gentamicin, and then cultured in the incubator for 24 hours. After 24 hours of culture, the entire culture medium was collected, transferred to a 50 mL tube, and centrifuged at 300 RCF for 3 minutes. The supernatant was carefully transferred to a new tube and used as conditioned media.

### 1-2. Cytokine Analysis and Liquid Chromatography Analysis

Cytokine analysis was performed using the conditioned media obtained from stem cells in Example 1-1 to verify the secreted proteins (i.e., protein expression). The analysis was conducted using the R & D Proteome Profiler Human XL Cytokine Array Kit and Raybio's Human Cytokine Array Kit, following the manufacturer's protocols.

Additionally, to cross-verify the potency factors in the conditioned media, protein quantification-based liquid chromatography analysis was outsourced to eBiogen Inc. (Korea), where the analysis was performed. The analysis process can be briefly described as follows. First, for the proteomics analysis of the conditioned media samples, a total of three runs were performed using three samples with the UPLC-Exactive equipment. For data analysis, the LC-MS/MS data for each sample were analyzed using Proteome Discoverer, with the Uniprot human database being used for the database search. The modifications analyzed included Oxidation (M), Carbamylation (N-term), and Carbamidomethylation (C). Among the identified proteins, sequences labeled as Con (Contaminants) and Rev (Reverse) were filtered out before the analysis.

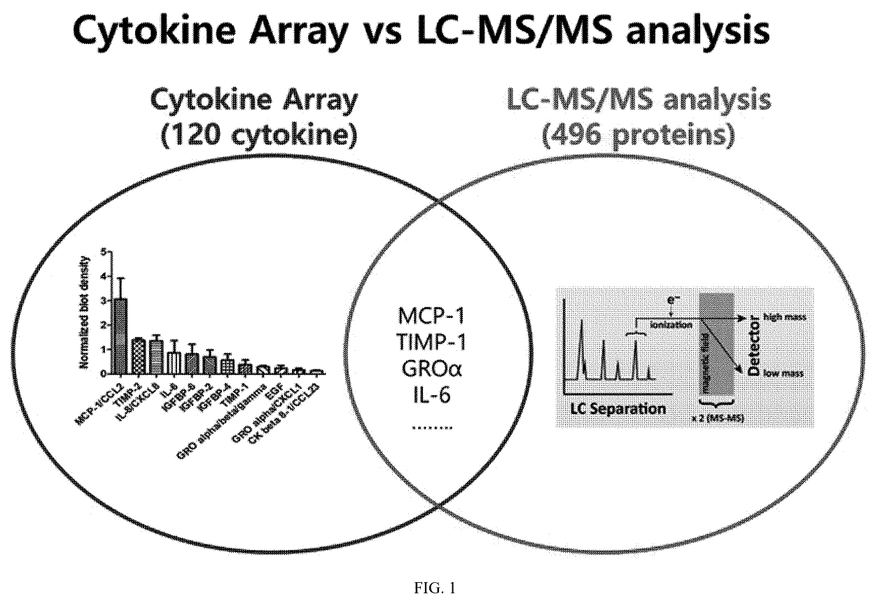

As briefly shown in FIG. 1, the cytokine analysis confirmed the expression levels of approximately 120 cytokines in the conditioned media, and the LC-MS/MS analysis revealed the presence of approximately 496 proteins. After numerous trials and errors, the inventors of the present invention successfully identified MCP-1, TIMP-1, GROα, and IL-6 as the efficacy factors among the many substances commonly present in the conditioned media.

The following examples specifically describe the superior therapeutic effects (prevention or treatment of neurological disease) of stem cells selected based on the specific combination of the four factors MCP-1, TIMP-1, GROα, and IL-6, and the stem cells selected according to these factors, in the context of the present invention using neural stem cells.

### 1-3. Selection of Highly Potent Neural Stem Cells for Treating Neurological Disease According to the Present Invention

According to the present invention, the protein concentrations of TIMP-1 (SEQ ID NO: 1), MCP-1 (SEQ ID NO: 2), GROα (SEQ ID NO: 3), and IL-6 (SEQ ID NO: 4) were measured in the stem cell conditioned media obtained from various human neural stem cell lines derived from the stem cell pool in Example 1-1. Neural stem cells containing high concentrations of these four factors were selected.

The specific method is as follows. First, 9,000 cells/cm² of neural stem cells (various human neural stem cell lines from the stem cell pool) were seeded in a T175 flask containing 35 mL of basic DMEM/F12 medium [Gibco (11330-032)] with 0.05% Gentamicin, and cultured in a CO2 incubator [Thermofisher (HERACELL VIOS 160i)] at 37.0° C. with 5% CO2 for 24 hours. After 24 hours of culture, the culture medium was collected in its entirety, transferred to a 50 mL tube, and centrifuged at 300 RCF for 3 minutes. The supernatant was carefully transferred to a new tube and used as the conditioned media.

The concentrations (protein amounts) of TIMP-1 (SEQ ID NO: 1), MCP-1 (SEQ ID NO: 2), GROα (SEQ ID NO: 3), and IL-6 (SEQ ID NO: 4) in the conditioned media were quantitatively measured using the Enzyme-Linked Immunosorbent Assay (ELISA) method. ELISA (Enzyme-Linked Immunosorbent Assay) is a method for measuring protein amounts by labeling antibodies or antigens with an enzyme as a marker and quantitatively measuring the enzyme's activity. This allows for the quantification of the strength and amount of the antigen-antibody reaction. The ELISA tests were performed using the following kits: Human MCP-1 Quantikine ELISA (R & D Systems, Cat no. DCP00), Human TIMP-1 Quantikine ELISA (R & D Systems, Cat no. DTM100), Human CXCL1/GROα DuoSet ELISA (R & D Systems, Cat no. DY275-05), and Human IL-6 DuoSet ELISA (R & D Systems, Cat no. DY970-05). The tests were carried out according to the manufacturer's instructions for each respective kit.

In the conditioned media, the concentrations of the four proteins were measured, and stem cells (i.e., stem cells expressing the four protein factors) were identified and selected as high-efficacy neural stem cells when the following conditions were simultaneously satisfied: TIMP-1 concentration of 6,000 μg/mL or higher, MCP-1 concentration of 6,000 μg/mL or higher, GROα concentration of 500 μg/mL or higher, and IL-6 concentration of 50 μg/mL or higher.

Using the above method, the NSC #1, NSC #2, and NSC #3 cell lines were successfully selected as high-efficacy neural stem cells. Using each of the NSC #1, NSC #2, or NSC #3 neural stem cells, conditioned media were prepared in three separate lots using the same method. These were used in the following experiments.

### Example 2. Creation of In Vitro Stroke Model

2-1. Isolation and Culturing of Primary Cortical Neurons from Rats

The pregnant rat (E18, OriGenebio) was euthanized using CO2 gas, and then the embryos were extracted through a cesarean section using surgical scissors. After extracting the embryos from the placenta sacs, the embryo skull was removed using forceps, and the brainstem and cerebellum tissues were carefully dissected under a microscope. After removing the meninges covering each hemisphere and excising the hippocampus, the cortical tissue was obtained. Then, 10 mL of 1×HBSS was added, and the tissue was finely minced by pipetting with a 10 mL serological pipette. After centrifuging the tissue to pellet it, the supernatant was discarded. Then, 3 mL of 1×HBSS was added, followed by pipetting. Next, Trypsin-EDTA was added, and the mixture was pipetted again before being incubated in a 37° C. water bath for 2 minutes. After centrifugation, the supernatant was discarded, and the pellet was resuspended in DMEM (containing 10% FBS and 1% P/S). The suspension was then filtered through a strainer to obtain a single-cell suspension, which was collected in a 50 ml conical tube. After counting the number of isolated cells, 1×10⁵ cells per well were seeded onto a PDL-coated plate containing DMEM (with 10% FBS and 1% P/S). After 4 hours, a half media change was performed using complete cortical neuron culture media (Neurobasal Media [Gibco (21103-049)] containing 2% B27 Supplement [50×, Serum Free, Gibco (17504-044)], L-Glutamine, and 1% Penicillin/Streptomycin [P/S]). Another half media change was performed with the same media 12 hours later. Following this, the cells were cultured for 3 days with full media changes once a day.

### 2-2. Creation of In Vitro Stroke Model (OGD Injury Condition)

The brain neurons isolated in Example 2-1 were seeded onto a 96-well plate and cultured for 7 days using a growth medium to allow the cells to mature into neurons. Subsequently, a medium without glucose was added, and the cells were exposed to 1% oxygen and glucose deprivation (OGD) conditions for 1 hour, thereby establishing an in vitro stroke model.

### Example 3. Analysis of Neuroprotective Effect of Neural Stem Cell Conditioned Media in In Vitro Stroke Model

The inhibitory effect on in vitro neuronal cell death by treatment with the conditioned media from the selected high-potency neural stem cells according to the present invention was analyzed. Specifically, after treating the in vitro stroke model established in Example 2-2 with the conditioned media from the neural stem cells obtained in Example 1-3, neuronal cell survival was measured through MTT assay. As a control group, growth media (GM, 100 μl) without OGD treatment was used as the positive control. Additionally, analysis was conducted using incomplete media (IM, 100 μl) as described in Example 1-1, and conditioned media (CM, 100 μl) obtained from the three lots of selected neural stem cells in Example 1-3.

First, after treating the in vitro stroke model (cells induced by OGD) established in Example 2-2 with the respective test substances for each control and experimental group, MTT Solution (Sigma, M5655) was applied at a concentration of 0.5 mg/ml. The plate was incubated for 1 hour, during which the formation of formazan was observed under a microscope. The media was removed, and 100 μl of DMSO was added to each well. The formazan was then dissolved in the DMSO for 30 minutes at 37° C. in a 5% CO2 incubator. The plate was placed in a microplate spectrometer (X-MARK Microplate Absorbance Spectrophotometer [Bio-rad (168-1150)]), and the optical density (O.D.) value was measured at a wavelength of 570 nm.

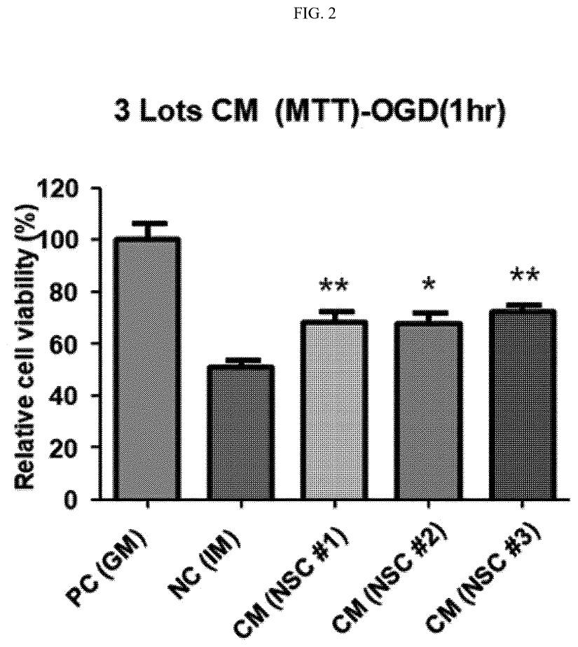

As shown in FIG. 2, compared to normal neuronal cells (PC, positive control), the stroke in vitro model (NC, negative control) exhibited a significant increase in neuronal cell death due to OGD induction. On the other hand, in the groups treated with the conditioned media from the three lots of neural stem cells under OGD damage conditions, a significant inhibition of neuronal cell death was observed.

This confirmed that the proteins secreted by neural stem cells exhibit excellent neuroprotective effects on neuronal cells.

### Example 4: Analysis of Changes in Neuroprotective Effects of Neural Stem Cell Conditioned Media in an In Vitro Stroke Model Upon Neutralizing Antibody Treatment of Effector Factors

To confirm whether the neuroprotective effect of the highly potent neural stem cell conditioned media, as observed in the in vitro stroke model (OGD-induced conditions) in Example 3, is regulated by the four combination effector factors of the present invention, after 1 hour of OGD-induced damage in the in vitro stroke model, the control and experimental groups were treated with IM (incomplete media, 100 μl), CM (conditioned media, 100 μl), and CM+neutralizing antibodies for each factor (10 μg/mL, 100 μl), respectively. After 24 hours of incubation, the neuroprotective effect was analyzed using the MTT assay (as described in Example 3).

The neutralizing antibody information for each factor used in this experiment is as follows: MCP-1 Ab [Santa Cruz (sc-32771)], TIMP-1 Ab [Santa Cruz (sc-21734)], GROα Ab [Santa Cruz (sc-514065)], IL-6 Ab [Santa Cruz (sc-57315)].

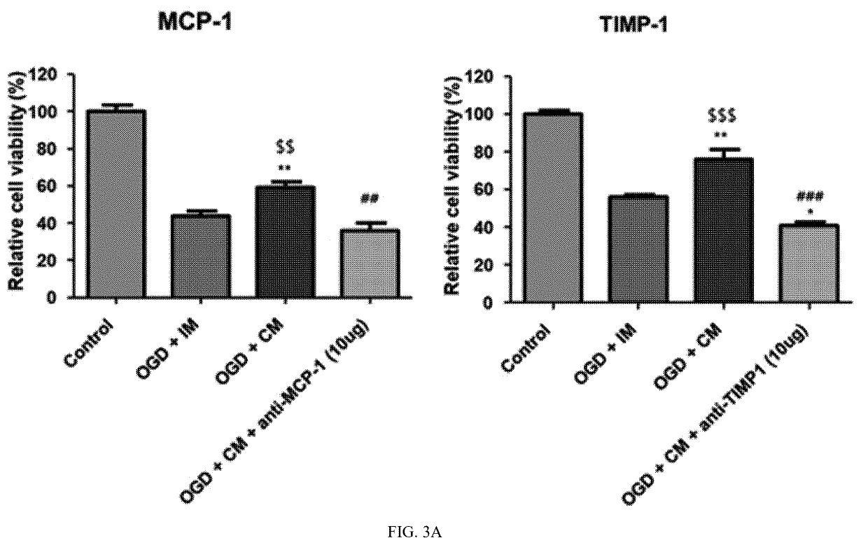

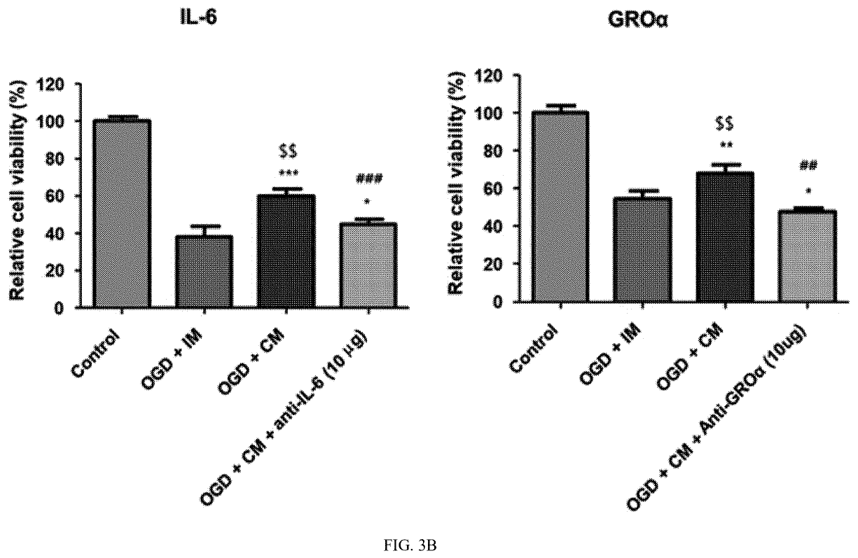

Experimental results demonstrated that, compared to normal cells (control), cell death was increased in the in vitro stroke model induced by the negative control OGD (OGD+IM). However, in the group treated with the conditioned medium (OGD+CM), cell death was significantly inhibited. When the conditioned medium was pre-incubated with 10 μg/well of neutralizing antibodies against MCP-1, TIMP-1, GROα, or IL-6 and then applied to neural cells, it was observed that the neuroprotective effect of the conditioned medium (i.e., its ability to inhibit neural cell death) was significantly reduced (FIGS. 3a and 3b).

Notably, even when only one of the factors MCP-1, TIMP-1, GROα, or IL-6 was inhibited using a neutralizing antibody, the extent of cell death was either greater than that of the negative control (OGD+IM) or similar to the level observed in the negative control, indicating significant cell death (FIGS. 3a and 3b). In other words, the neuroprotective ability (neuroprotection effect) was substantially lost if even one of the four factors was absent. This confirmed that the combination of the four factors of the present invention (MCP-1, TIMP-1, GROα, and IL-6) must be present simultaneously.

### Example 5. Analysis of the Neuroprotective Effect (Neuronal Cell Death Inhibition) of Neural Stem Cell-Conditioned Medium in an In Vitro Spinal Cord Injury Model

The neuroprotective effect of conditioned medium on spinal cord neurons subjected to cell death induced by reactive oxygen species (ROS), a secondary injury factor associated with spinal cord injury, was evaluated as follows. This analysis was performed in comparison to the results obtained in Example 3, where neural stem cell-conditioned medium was applied to an in vitro stroke model.

5-1. Isolation and Culture of Primary Spinal Cord Neurons (Motor Neurons) from Rats

After euthanizing a pregnant rat (E15, purchased from Orient Bio) using CO2 gas, a cesarean section was performed using surgical scissors to extract the embryos. The embryos were removed from the placenta sacs, and their bodies were stabilized with forceps. Using tweezers, the outer membrane of the spinal cord was carefully removed. The spinal cord was carefully separated from the Rat body after removing the outer membrane. The meninges surrounding the spinal cord and the dorsal root ganglia were then removed to isolate the pure spinal cord, which was transferred into a conical tube containing 15 ml of 1×HBS. The tissue was pelleted using a centrifuge, and the supernatant was removed. 10 ml of 1×HBSS was added to the tube, followed by gentle pipetting, and centrifugation was performed. After removing the supernatant, 3 ml of 1×HBSS was added, followed by pipetting. Then, Trypsin-EDTA was added, and the mixture was pipetted again before being placed in a 37° C. water bath for 2 minutes. After centrifugation, the supernatant was removed, and the pellet was resuspended in DMEM (containing 10% FBS and 1% P/S). The suspension was then passed through a 70 μm strainer attached to a 50 mL conical tube to separate it into single cells. The separated cells were counted and then seeded into plates. After at least 2 hours, it was confirmed that the cells had adhered to the plates. Subsequently, a half media change was performed using Complete Motor Neuron Culture Media, and this process was repeated once daily for three days. Afterward, a whole media change was performed every three days.

### 5-2. Creation of the In Vitro Spinal Cord Injury Model Induced by H2O2 and Analysis of the Neuroprotective Effect of Neural Stem Cell-Conditioned Media on Spinal Cord Neurons

The spinal cord neurons isolated in Example 5-1 were exposed to the secondary factor of spinal cord injury, ROS (H2O2, Hydrogen peroxide 0.1 mM), for 1 hour to establish an in vitro spinal cord injury model. Specifically, the spinal cord neurons isolated in Example 5-1 were cultured in GM (growth medium) and then switched to IM (basal medium) containing 0.1 mM H2O2 (Hydrogen peroxide) for 1 hour to establish an in vitro spinal cord injury model.

As a control, fresh IM (basal medium) was applied, and as the experimental group, CM (neural stem cell-conditioned medium obtained in Example 1-3) was applied. After 6 hours, the spinal cord neuron survival effect (neuroprotective ability) was analyzed using the MTT assay. The MTT assay was performed using the same method described in Example 3.

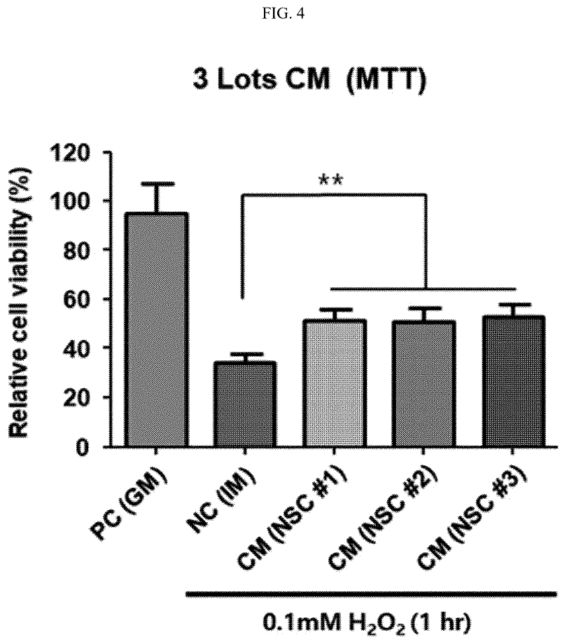

As shown in FIG. 4, compared to normal spinal cord neurons (PC, positive control), neuronal cell death significantly increased under spinal cord injury conditions induced by H2O2 treatment (NC, negative control). However, in the group treated with the neural stem cell-conditioned medium according to the present invention under the same injury conditions, neuronal cell death was markedly suppressed.

This confirms that the proteins secreted by neural stem cells not only protect brain neurons but also exhibit excellent protective effects on spinal cord neurons.

### Example 6. Analysis of Changes in Neuroprotective Effects Following Neutralizing Antibody Treatment of Efficacious Factors in Neural Stem Cell-Conditioned Medium in an In Vitro Spinal Cord Injury Model

To verify whether the neuroprotective effect of the conditioned medium observed in Example 5-2 is regulated by the four candidate efficacious factors of the present invention, neutralizing antibodies for each factor were added to the conditioned medium following the same method as described in Example 4. Changes in spinal cord neuronal cell death were then observed using the same method as in Example 5-2.

At this time, the results were also compared with those obtained from the treatment with a general IgG (10 μg/ml, IgG [Santa Cruz (sc-2025)]).

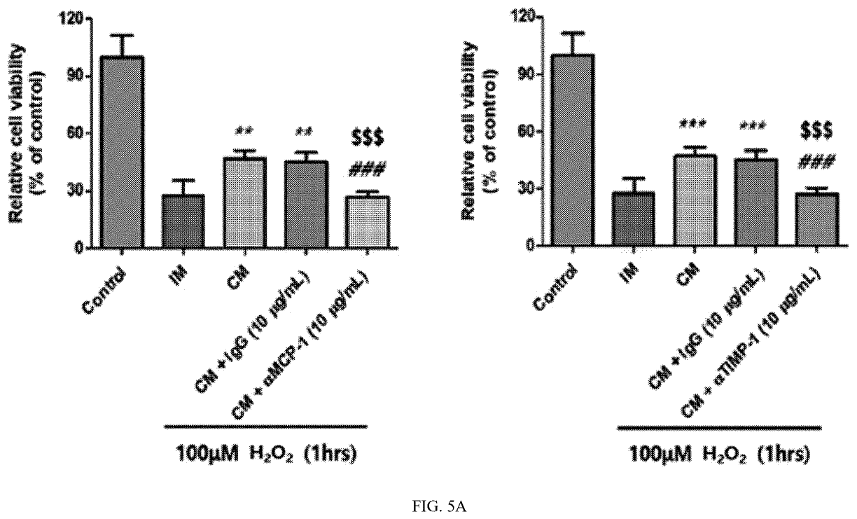

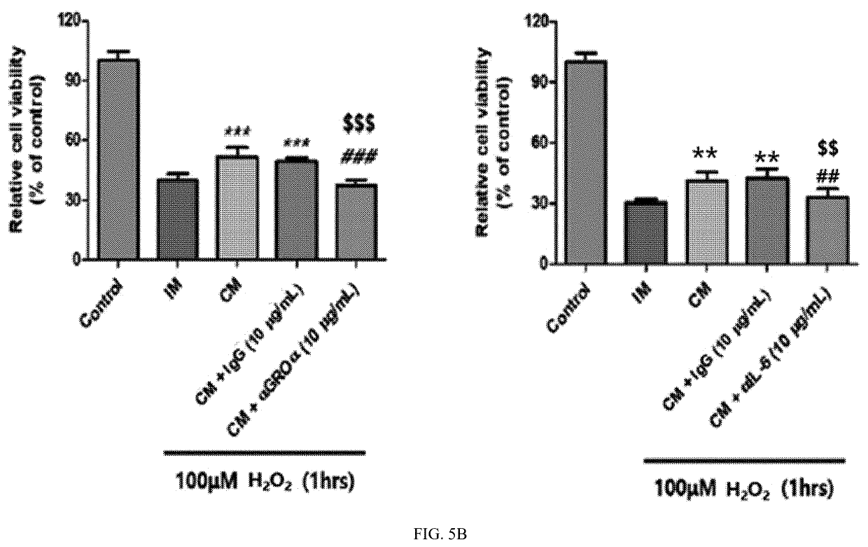

As shown in FIGS. 5a and 5b, in the ROS-induced spinal cord injury in vitro model (IM+H2O2), spinal cord neuron death significantly increased compared to the normal cell group (PC). In contrast, in the treatment group with the neural stem cell-conditioned media (CM, containing all four factors) of the present invention, cell death was markedly reduced. When conditioned media were pre-incubated with neutralizing antibodies against MCP-1, TIMP-1, GROα, or IL-6 and subsequently treated on spinal cord neurons, the spinal cord neuron death inhibition effect of the conditioned media was significantly reduced (FIGS. 5a and 5b). Specifically, even when only one of MCP-1, TIMP-1, GROα, or IL-6 was neutralized with an antibody, a similar level of cell death as the negative control group (IM) was observed (FIGS. 5a and 5b). This confirmed that the combination of four factors of the present invention (MCP-1, TIMP-1, GROα, and IL-6) plays a critical role in suppressing spinal cord neuron death in the conditioned medium.

### Example 7. Effect of Neural Stem Cell-Conditioned Medium on Vascular Cell Migration Ability in Endothelial Cells

In angiogenesis, the migration of endothelial cells is an essential step in the process of forming blood vessels, occurring in conjunction with the degradation of extracellular matrix components surrounding the vasculature. Using HUVECs (Human Umbilical Vein Endothelial Cells), the change in endothelial cell migration ability induced by the neural stem cell-conditioned medium obtained in Example 1-3 was analyzed through the wound-healing assay method. HUVECs were seeded into a 12-well plate using Endothelial Cell Growth Medium 2 [PromoCell (C-22211)] supplemented with Growth Medium 2 Supplement Mix [PromoCell (C-39216), 2.5%] and Penicillin-Streptomycin (P/S, 1%). The cells were then cultured in a 5% CO₂, 37° C. incubator for 16 hours. After culturing the cells to 90% confluence, the supernatant was removed using a suction system. Subsequently, 2 mL of IM (Incomplete Media, Endothelial Cell Growth Medium 2 [PromoCell (C-22211)] supplemented with Penicillin-Streptomycin (P/S, 1%)) was added, and the cells were subjected to starvation (cell nutrient deprivation) for 7 hours. Using a 200 μL pipette tip, a cross-shaped scratch was made in each well. The supernatant was then removed using a suction system, and 1.5 mL of CM (Conditioned Media) was added to each well. Images of the cross-shaped area were captured under a microscope, focusing on the center of the scratch along both vertical and horizontal axes. After 17 hours, images were captured at the same locations as the initial photographs. The distance between the edges of the scratch was measured, and the difference in distance between 0 hours and 17 hours was calculated and converted into a percentage. These data were then organized into a graph.

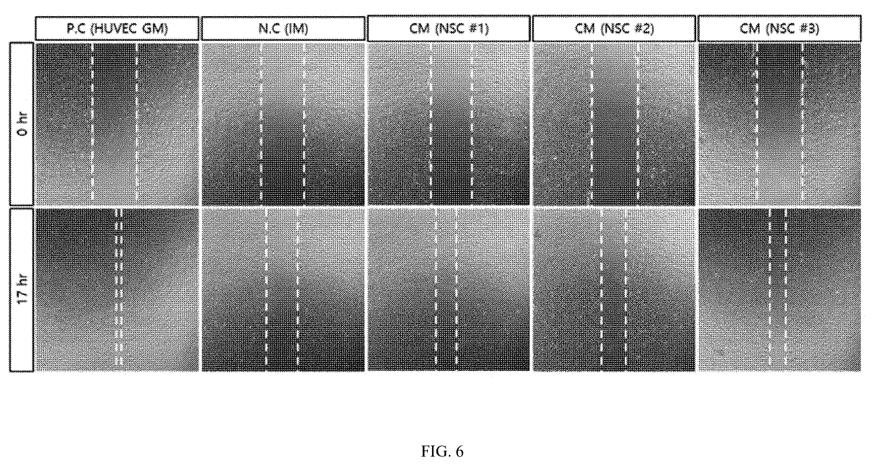

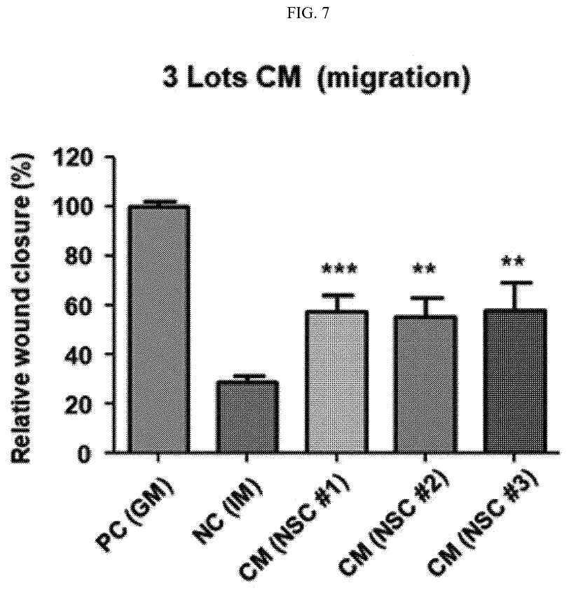

As shown in FIGS. 6 and 7, compared to IM (negative control, NC), the group treated with the neural stem cell-conditioned medium (repeated in three separate lots) exhibited a significantly enhanced migration of endothelial cells.

### Example 8. Analysis of Changes in Endothelial Cell Migration Inhibition by Neutralizing Antibodies Against Functional Factors in Neural Stem Cell-Conditioned Medium

To determine whether the increase in endothelial cell (HUVEC) migration observed in Example 7 through the conditioned medium is regulated by the four functional factors of the present invention, neutralizing antibodies against each factor were added to the conditioned medium following the same method as described in Example 4. The changes in endothelial cell migration ability were analyzed using the same method as in Example 7.

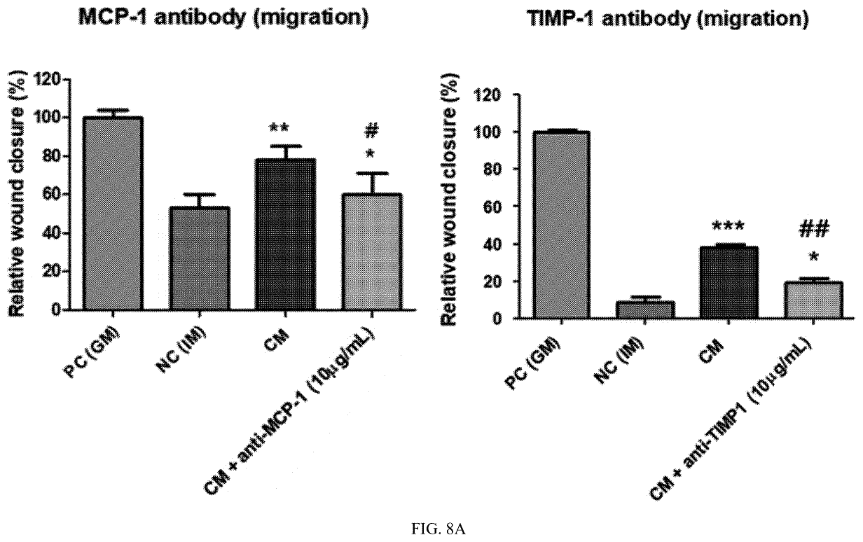

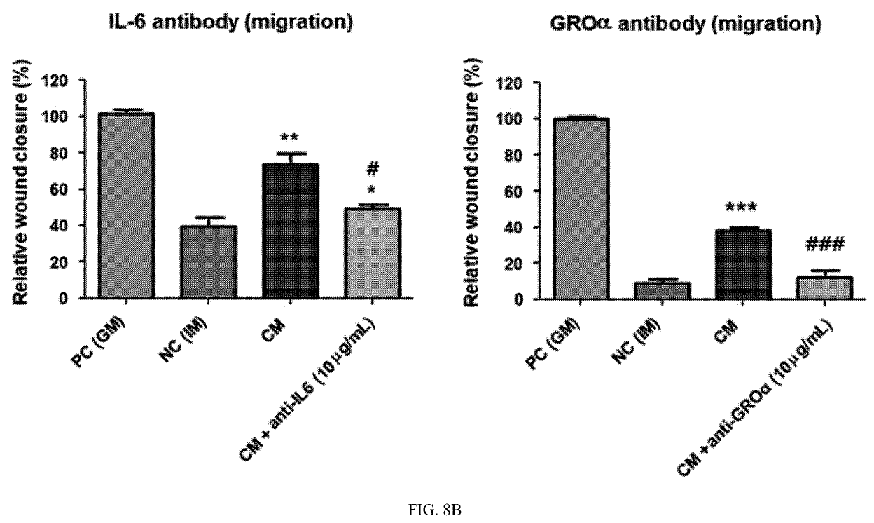

As shown in FIGS. 8a and 8b, treatment of the conditioned medium with neutralizing antibodies against MCP-1, TIMP-1, GROα, or IL-6 significantly reduced endothelial cell migration compared to the group treated with the conditioned medium (CM). In other words, the migration ability of endothelial cells was substantially lost when any one of the four factors was absent. This verified that the combination of four factors (MCP-1, TIMP-1, GROα, and IL-6) in the present invention is essential to be present simultaneously.

### Example 9. Evaluation of Tubulogenesis Ability of Endothelial Cells Induced by Neural Stem Cell Conditioned Media Treatment

In a broad sense, angiogenesis refers to the series of processes through which new blood vessels are formed. This involves the proliferation and migration of cells constituting pre-existing blood vessels, ultimately creating new tubular structures that play a vital role in supplying nutrients and blood. This process is closely related to the regeneration of damaged tissues and can serve as an important indicator for evaluating overall therapeutic efficacy. To verify the tube formation ability of vascular cells induced by proteins secreted from neural stem cells, the neural stem cell-conditioned medium was applied to vascular endothelial cells to assess whether tube formation could be enhanced.

To evaluate the tube formation ability of vascular endothelial cells induced by the neural stem cell-conditioned medium, Matrigel was used. After coating a 96-well plate with Matrigel, 1×10⁴ HUVEC cells were mixed with culture medium and dispensed into the wells. After 10 minutes, the attachment of the cells was confirmed, and the culture medium was replaced with IM (Incomplete Media) to induce cell starvation for 2 hours. The medium was then replaced with CM (Conditioned Media), and microscope images were captured (0 h). After 20 hours, microscope images were taken for each sample, and the total length of the formed tubes was measured using the ImageJ program. The average and standard deviation were calculated, and the results were summarized in a graph.

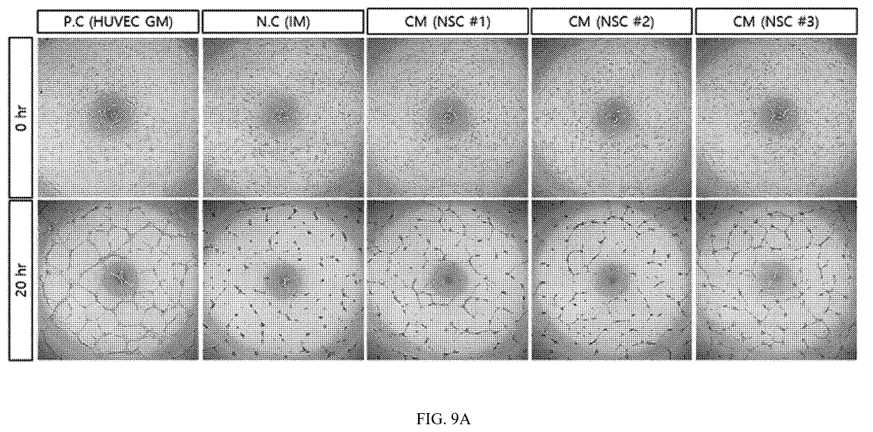

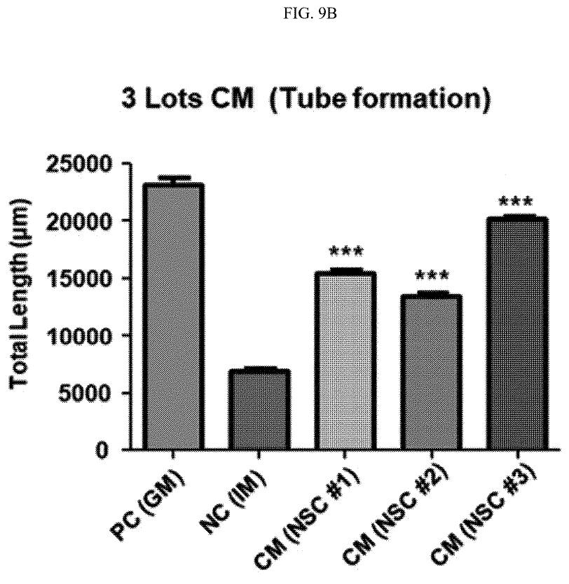

As shown in FIGS. 9a and 9b, it was confirmed that when the neural stem cell-conditioned media according to the present invention was applied, the tube formation ability of endothelial cells was significantly enhanced.

### Example 10. Analysis of Changes in Tube Formation Ability of Endothelial Cells Following Neutralizing Antibody Treatment of Efficacy Factors in Neural Stem Cell-Conditioned Media

To verify whether the increased tube formation ability of endothelial cells induced by the conditioned media, as confirmed in Example 9, can be regulated by the four combinatorial efficacy factors of the present invention, each neutralizing antibody targeting the individual factors was treated in the conditioned media following the same method as described in Example 4. Subsequently, changes in the tube formation ability of endothelial cells were observed using the same method as described in Example 9.

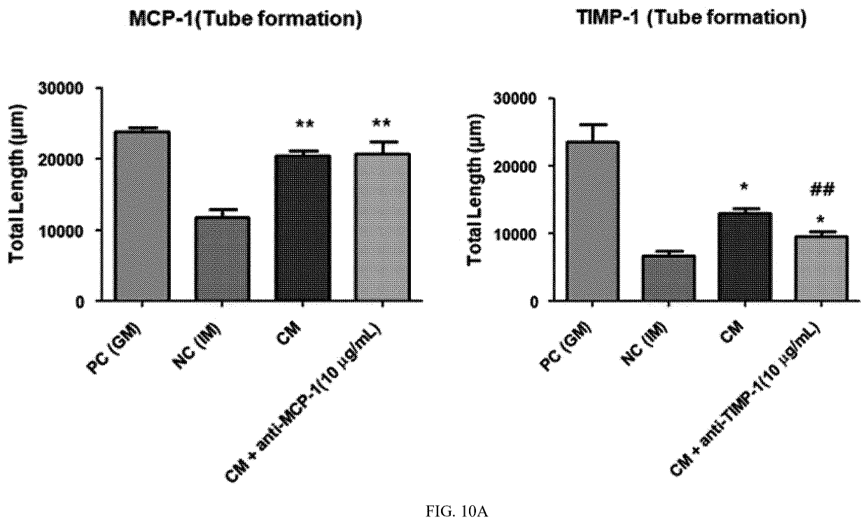

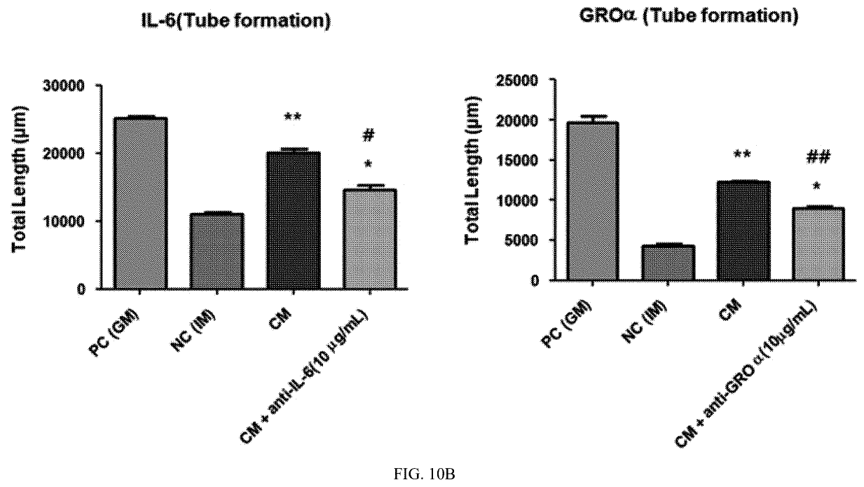

As shown in FIGS. 10a and 10b, compared to the group treated with the conditioned media (CM), the tube formation ability of endothelial cells was significantly reduced in the groups where neutralizing antibodies for TIMP-1, GROα, or IL-6 were added to the conditioned media.

In summary, the combination of the four factors of the present invention (MCP-1, TIMP-1, GROα, and IL-6) plays a critical role in regulating the migratory ability of endothelial cells, and it was confirmed and validated that these factors can enhance the tube formation ability of endothelial cells (particularly TIMP-1, GROα, and IL-6). In other words, the migratory ability of endothelial cells is significantly reduced if any one of the four factors is absent. This validates that the combination of the four factors (MCP-1, TIMP-1, GROα, and IL-6) of the present invention must be present simultaneously. These results confirm the efficacy of the factors as agents playing a crucial role in the regeneration and protection of damaged neural tissue.

### Example 11. Evaluation of Behavioral Improvement by the Injection of Selected Neural Stem Cells According to the Present Invention in an In Vivo Spinal Cord Injury Animal Model

In order to perform a behavioral evaluation following the injection of neural stem cells in an in vivo spinal cord injury animal model, the spinal cord injury model was created and the test was conducted using the following method. Approximately 50 ml of Isoflurane mixed with oxygen (O₂) was used to induce inhalation anesthesia in 10-week-old rats. After exposing the thoracic vertebrae (T8-T10), the muscles attached to the spine were separated, and a laminectomy was performed to remove the lamina of the T9 vertebra. Subsequently, an impactor was used to apply pressure to the dorsal dura mater of the spinal cord to create the spinal cord injury model. Seven days later, in the spinal cord injury model, the control group (G1) was administered a vehicle, while 1×10² (G3) or 3×10² (G2) neural stem cells (highly potent neural stem cells selected according to the present invention) were transplanted into the spinal cord cavity. After the transplantation of neural stem cells, neurological behavioral assessments and neuropathic pain evaluations were conducted weekly. For the neurological behavioral assessment, the BBB score test (Basso, Beattie, and Bresnahan score test) and rota-rod test were performed. For the neuropathic pain evaluation, the von Frey test was conducted.

Specifically, the BBB score test was conducted a total of 7 times, with assessments performed before the administration of the test substance and at weekly intervals thereafter. The scoring of the test was based on the method evaluated by DM Basso et al. (J Neurotrauma. 1995 February; 12(1):1-21).

The rotarod test was conducted a total of 7 times, with assessments performed before the administration of the test substance and at weekly intervals thereafter, using the Rotarod latency test. The animals were carefully placed on a Rotarod treadmill (JD-A-07RA5, B.S Technolab Inc., Korea), and the rotation speed was set to increase in a steady interval from 4 rpm to 40 rpm over a period of 300 seconds. The time until the animal lost balance and fell to the floor was measured in seconds. If the animal fell to the floor, the time was automatically measured using a sensor located on the floor of the Rotarod treadmill. For each animal, after measuring once, measurements were repeated once more for a total of three times, and the average value was used for the test.

The von Frey test was performed before the administration of the test substance and then once a week thereafter for a total of seven times. After stabilizing the animals in a plastic box with a fine metal mesh on the floor to allow them to acclimate to the environment, pain levels were assessed by observing the withdrawal response to mechanical stimulation using von Frey filaments. Mechanical stimulation was applied to the animal's paw through the holes in the wire mesh floor of the box using a von Frey filament. A positive response was defined as the animal sharply withdrawing its foot, scratching, or flinching in response to the filament stimulus. The strength of the filament at the time of the positive response was then measured. To ensure accurate measurement, pressure was applied within approximately 3-5 seconds until the von Frey filament bent. The filament strength was increased in the following order: 0.6, 1, 1.4, 2, 4, 6, 8, 10, 15, 26, 60, 100, 180, and 300 g, until a positive response was observed. For each animal, one measurement was taken initially, followed by additional measurements at one-week intervals for a total of three measurements, and the average value was used for the test.

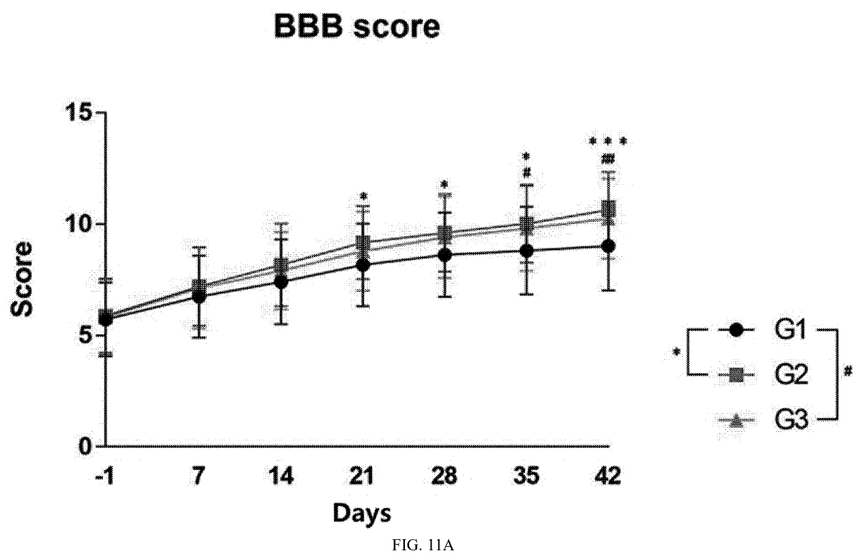

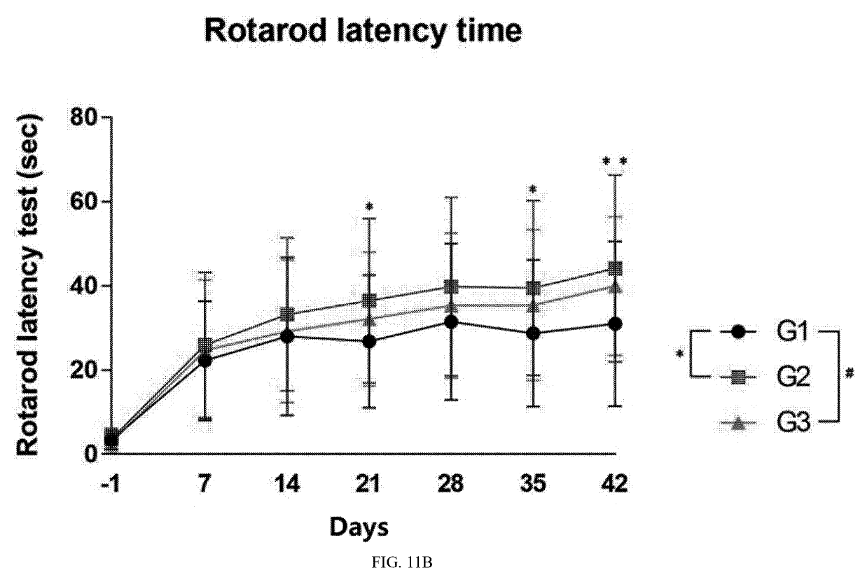

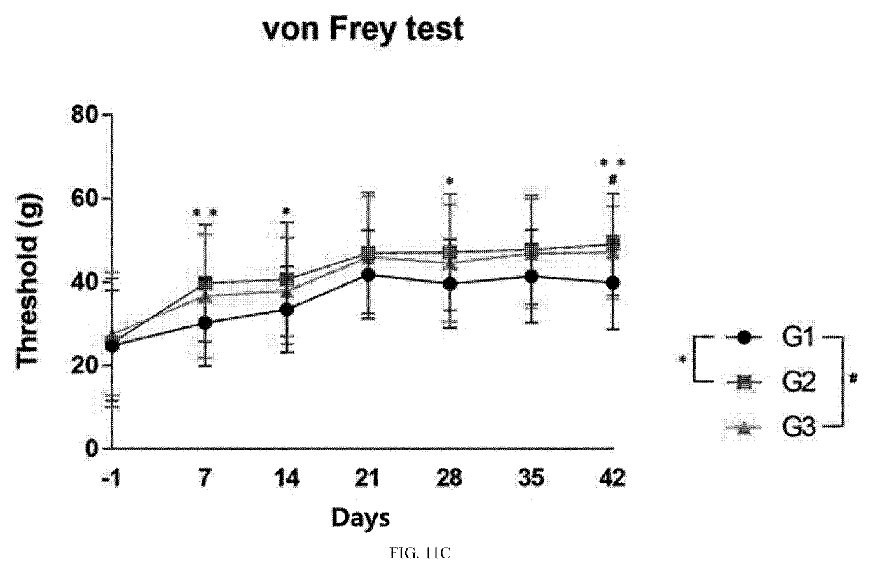

As shown in FIG. 11a and FIG. 11b, the results of the BBB behavioral score test and the rota-rod test evaluating motor ability confirmed that the G2 and G3 groups, which received the high-potency neural stem cells selected according to the present invention, showed significant improvements in behavior compared to the vehicle control group (G1). Moreover, it was observed that this difference in effect became even more pronounced after 5 weeks post-transplantation of the neural stem cells. Additionally, as shown in FIG. 11c, the results of the neuropathic pain assessment (von Frey test) demonstrated significant improvement in the G2 and G3 groups, which received the high-potency neural stem cells selected according to the present invention, starting from one week post-transplantation, showing a marked effect on pain alleviation.

As a result, it was confirmed and verified that the neural stem cells selected according to the present invention can improve behavioral impairments caused by spinal cord injury. However, no significant difference was observed between the G2 and G3 groups with an increased number of neural stem cell injections (transplantations).

### Example 12. Verification of the Effect of Reduced Brain Tissue Shrinkage (Damage) Following the Injection of Neural Stem Cells Selected According to the Present Invention in an In Vivo Ischemic Stroke Animal Model (Middle Cerebral Artery Occlusion Rat Model, MCAO)

When the neural stem cells selected according to the present invention were injected into an in vivo ischemic stroke animal model, the following experiment was conducted to evaluate the therapeutic effect of the disease and the degree of improvement in brain tissue damage.

### 12-1. Establishment of MCAO Ischemic Stroke Animal Model

After anesthetizing 8-week-old Sprague-Dawley (SD) rats with a mixture of isoflurane and oxygen, the skin of the neck was shaved to expose the area. After disinfecting with an alcohol swab, a 3 cm incision was made along the neck midline using operating scissors. After fixing the salivary glands toward the trachea, the muscle tissue was grasped with curved Crile forceps to expose the common carotid artery (CCA), external carotid artery (ECA), and internal carotid artery (ICA). The common carotid artery (CCA) was exposed using curved iris forceps, and a silk suture was looped around it. Similarly, the external carotid artery (ECA) and internal carotid artery (ICA) were exposed, and a suture was looped around them. The ligatures around the CCA and ECA, excluding the ICA, were tightened to secure them in place. A syringe was used to create a small hole at the junction where the common carotid artery (CCA), external carotid artery (ECA), and internal carotid artery (ICA) meet. A silicon-coated MCAO catheter was then inserted from the bifurcation of the CCA toward the ICA. The ICA was secured by tying it together with the MCAO catheter. The salivary gland was repositioned, and the incised skin was sutured closed. One hour after the occlusion, the same method was followed: under anesthesia, the CCA, ECA, and ICA were exposed, the previously fixed ligatures were released, and the inserted MCAO suture was removed to initiate reperfusion. After achieving hemostasis, the skin was sutured. One day after reperfusion, neurological symptoms were evaluated to confirm the presence of cerebral infarction.

### 12-2. Experimental Method for Confirming Therapeutic Effect of MCAO

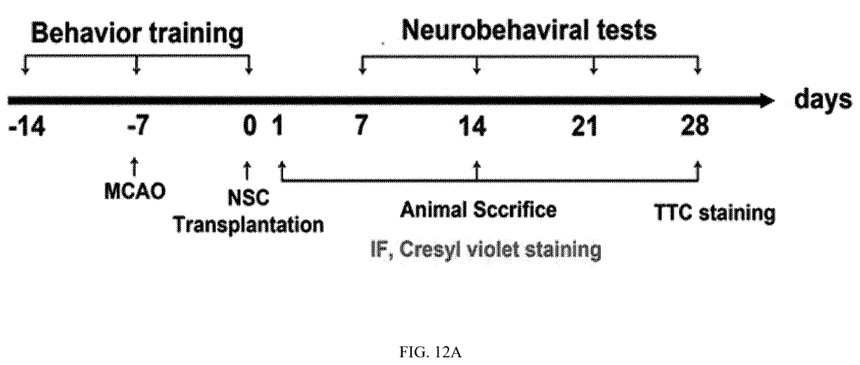

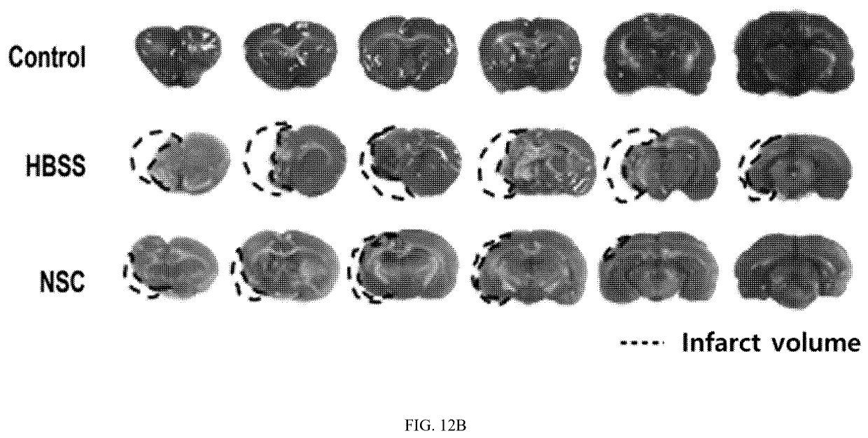

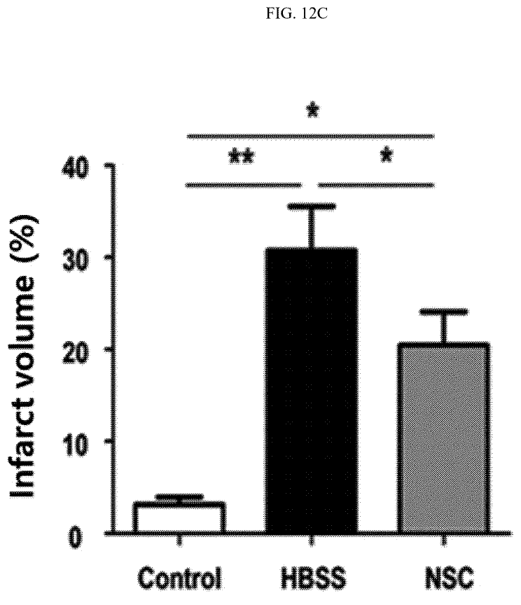

Ischemic Stroke Using Stem Cells Selected According to the present invention Seven days after the creation of the MCAO ischemic stroke animal model as described in Example 12-1, 5×10⁵ highly potent neural stem cells selected according to the present invention were transplanted into the ventricles. The experimental protocol for neurological behavioral evaluation and tissue analysis on the above animal model is briefly outlined in the timeline shown in FIG. 12a. For the three groups-Control (Wild type normal rats), MCAO+HBSS (Hanks Balanced Salt Solution, 20 μL 1×HBSS), and MCAO+NSC (highly potent neural stem cells selected according to the present invention, 5×10⁵ cells suspended in 20 μL 1×HBSS)-neurological behavioral evaluations were conducted one week before the MCAO surgery, one week before HBSS or NSC transplantation, and then at 1, 2, 3, and 4 weeks post-transplantation. The three groups were sacrificed on days 1, 14, and 28 post-transplantation. Tissue samples were prepared, followed by immunohistochemical staining and immunofluorescence (IF). On day 28, Triphenyltetrazolium chloride (TTC) staining was also performed. Specific details of the methods are provided in each of the respective examples.

### 12-3. Confirmation of Brain Injury Treatment Effect in MCAO Ischemic Stroke Model Following Administration of the Neural Stem Cells Selected According to the Present Invention

Brain tissue obtained from control and experimental group rats was sectioned using a brain matrix, with 2 mm intervals along the frontal pole to the parietal surface, to prepare tissue slices. The brain tissue slices were stained by incubating them in 2% 2,3,5-triphenyltetrazolium chloride (TTC) solution (in phosphate-buffered saline, PBS) at 37° C. for one hour. The stained tissue slices were placed in 4% paraformaldehyde solution (in PBS) and fixed at 4° C. for one day. Subsequently, the brain tissue was sequentially placed on OHP film, and photographs were taken. The infarct volume of the brain tissue slices was calculated using the following percentage (%) calculation formula. The infarct volume was measured using the ImageJ program (National Institutes of Health, USA).

Infarct volume (% of Infarct volume)={(volume of contralateral hemisphere−volume of non-ischemic ipsilateral hemisphere)/volume of contralateral hemisphere}×100

The results of TTC staining conducted 28 days after the injection of the stem cells selected according to the present invention showed a significant and remarkable reduction in the infarct volume in the group injected with the highly potent neural stem cells, compared to the tissue damage area in the HBSS-treated group (FIG. 12b and FIG. 12c). As a result, it was confirmed and verified that the transplantation of highly potent neural stem cells selected according to the present invention reduces the infarct area in ischemic stroke.

### Example 13. Evaluation of Behavioral Improvement by Administration of Neural Stem Cells Selected According to the Present Invention in an In Vivo Ischemic Stroke Animal Model (Middle Cerebral Occlusion Rat Model, MCAO)

In the animal experiment conducted according to the protocol of Example 12-2, neurological behavioral assessments were performed at one-week intervals after the administration of neural stem cells selected according to the present invention to evaluate the effect of the neural stem cells on the improvement of brain damage.

The rota-rod test is a measure used to assess the balance and motor coordination of experimental animals. The experimental animals were trained three times daily starting three days prior to the MCAO surgery, and animals that showed good learning performance were selected to create the MCAO model. The animal trials were then performed according to the protocol outlined in Example 12-2. Then, the experimental animals were placed on a rota-rod treadmill that accelerated from 4 to 40 rpm, and the time until they fell off was measured. The maximum observation time for each experiment was set to 3 minutes. The measurement is terminated under the following conditions: 1) when the experimental animal falls off the treadmill completely, and 2) when the experimental animal rotates continuously for more than three laps while holding onto the treadmill. The measurement is re-conducted under the following conditions: 1) when the experimental animal runs in the opposite direction, and 2) when the experimental animal jumps off the treadmill or behaves distractedly, not walking properly. If the same result is obtained upon re-measurement, the animal is excluded from the group.

The Passive Avoidance Test is a measure used to evaluate the short-term and long-term memory of experimental animals. The Passive Avoidance Test was conducted by performing a training trial followed by a memory test trial the next day. In the training trial, the animals were allowed to habituate to the chamber for 30 seconds without illumination, after which the light was turned on. After a 15-second acclimatization period, the door allowing the test animal to move into the dark chamber was opened for 300 seconds. When the test animal moved into the dark chamber, the door was closed, and a 15-second acclimatization period was given, followed by a 15-second electric shock at 0.3 mA. If the test animal did not move, it was observed until it eventually moved. On the following day, a memory test was conducted to evaluate memory and cognition. During this test, no electric shock was applied to avoid stressing the animal. The memory test was performed under the same conditions as the learning test to ensure consistency. In the learning test, the time taken for the animal to move to the dark chamber after the light was turned on was measured.

The Adhesive Removal Test is a measure used to evaluate tactile responses and asymmetry in experimental animals. Paper dot stickers (9 mm in diameter) were attached to the forepaws of the experimental animals, and the time taken for the animals to recognize and remove the stickers was measured. The measurements were taken separately for the right and left front paws, with the maximum duration for each experiment set to 1 minute. The procedure was repeated three times, and the average was calculated (values falling outside the median ±50% of the raw data were excluded).

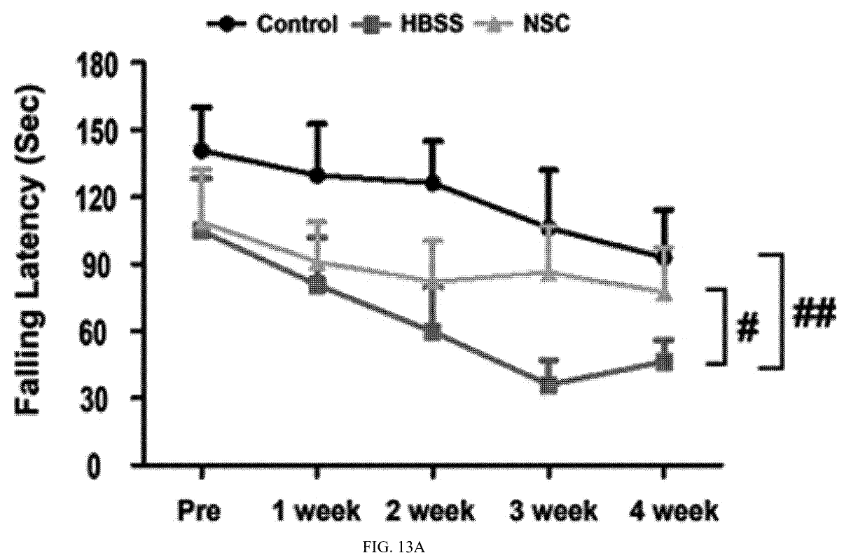

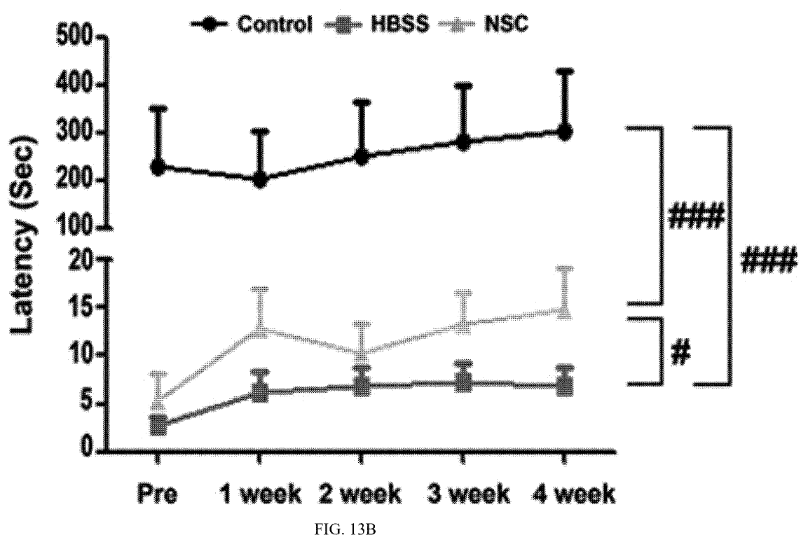

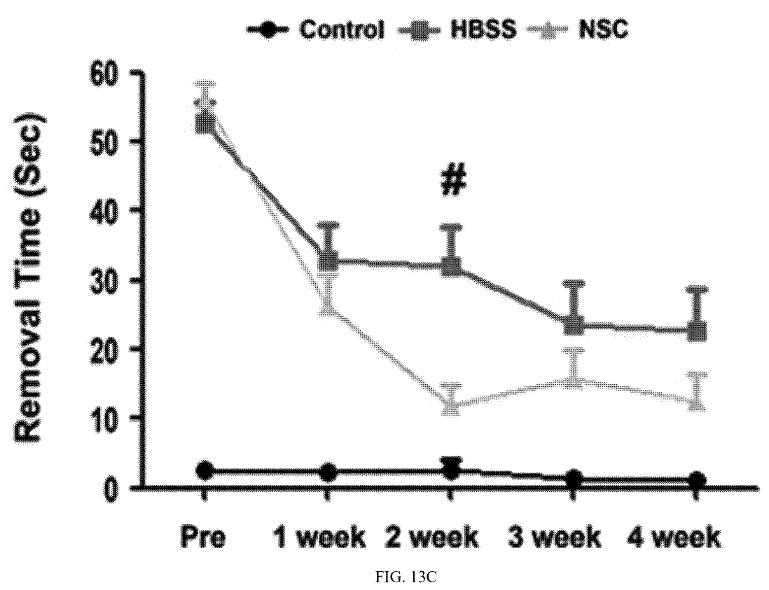

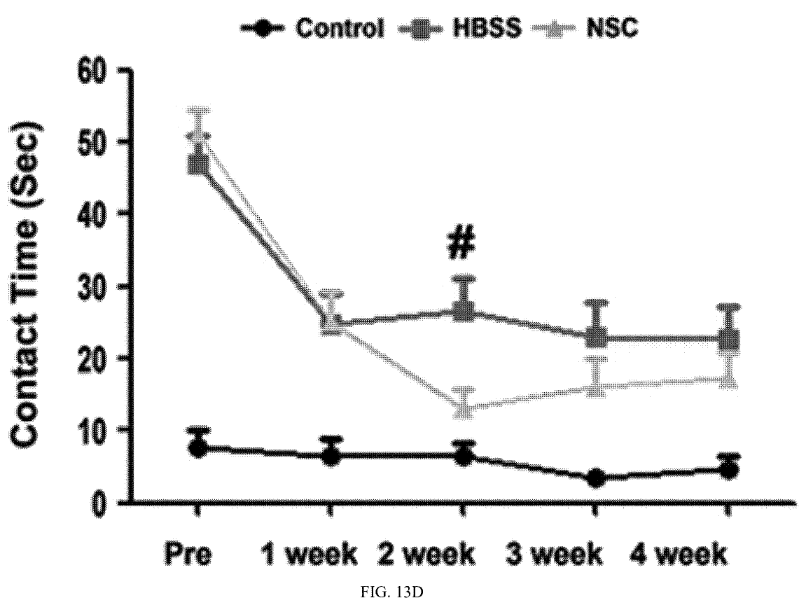

As shown in FIG. 13a, the results of the rota-rod test, which was conducted to assess motor function, revealed a significant improvement in motor ability in the highly potent neural stem cell injection group according to the present invention, compared to the HBSS injection group.

As shown in FIG. 13b, the results of the passive avoidance test, which was conducted to evaluate cognitive and memory abilities, confirmed that the group injected with highly potent neural stem cells according to the present invention showed an increase in cognitive memory ability compared to the group injected with HBSS.

As shown in FIGS. 13c and 13d, the results of the adhesive removal test, which was conducted to evaluate sensory and motor abilities, confirmed that the sensory and motor functions of the injured forelimb were significantly improved in the highly potent neural stem cell transplantation group according to the present invention, with a particularly significant improvement observed at the 2-week mark. In other words, it was confirmed that the neurobehavioral deficits were markedly alleviated in the highly potent neural stem cell transplantation group according to the present invention.

### Example 14. Analysis of Histological Improvement Effect (Neuronal Expression) by Injection of Selected Neural Stem Cells According to the Present Invention in an In Vivo Ischemic Stroke Animal Model (Middle Cerebral Occlusion Rat Model, MCAO)

In the animal experiment performed according to the protocol of Example 12-2, brain tissue was collected, fixed with 4% PFA using conventional methods, and paraffin blocks were prepared. To perform various immunofluorescence staining, tissue sections were made with a thickness of 5 μm, placed on slide glasses, and underwent a deparaffinization process before performing NeuN staining to evaluate the increase or decrease of neuronal cells.

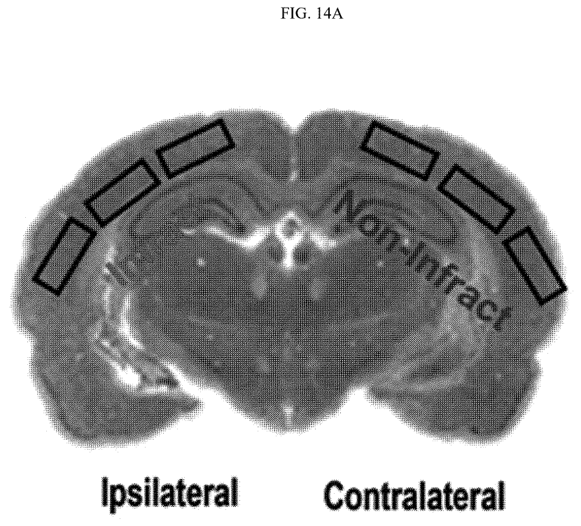

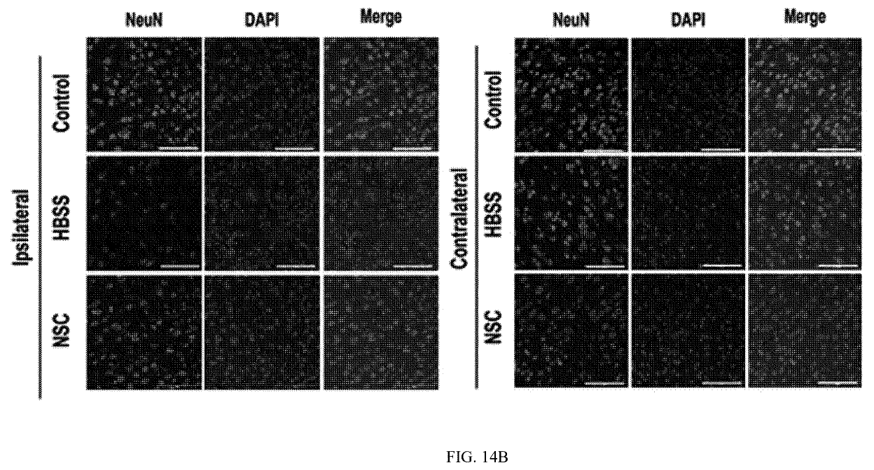

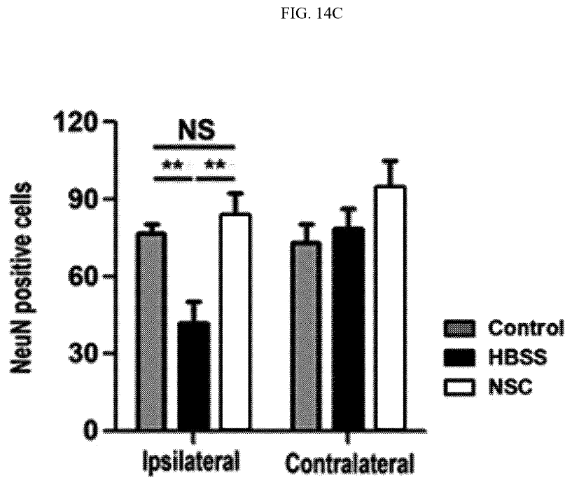

The specific method is as follows. First, the section slides of rat brain tissue that were frozen were washed three times with 1×PBS for 5 minutes each. The tissue samples were incubated at room temperature for 30 minutes with 50% ethanol, followed by treatment with 2.5% normal goat serum (VECTOR, S-1012) for 1 hour. The samples were treated with the primary antibody, NeuN Ab (dilution: 1:100, [abcam (ab177487)]), and incubated overnight at 4° C. Before the secondary antibody was applied, the samples were incubated for 15 minutes with 1% normal goat serum. Afterwards, the samples were incubated at room temperature for 2 hours with the secondary antibody. The secondary antibody, Goat anti-Rabbit IgG (H+L) Alexa Fluor 594, was diluted at a ratio of 1:500 in 1×PBS and used for the incubation. The 4′,6-diamidino-2-phenylindole (DAPI) solution (SIGMA, D9564) was diluted 1:100,000 in PBS and incubated for 5 minutes. Finally, the immunofluorescently stained brain tissue slides were mounted with VECTASHIELD mounting solution (VECTOR Laboratories, H-1400) and covered with a coverslip (Deckgloser, 24×32 mm). Then, using a confocal laser microscope (Leica) at a magnification of 400×, three regions were observed: the ipsilateral area of the cortical infarct region and the non-infarct regions in the contralateral area, which were randomly selected. The tissue regions observed in the damaged ipsilateral area and the undamaged contralateral area are shown in FIG. 14a.

As shown in FIG. 14b and FIG. 14c, the results confirmed that the number of neurons decreased in the group injected with HBSS compared to the Control group. Compared to the HBSS group, the group implanted with highly potent neural stem cells according to the present invention showed a significant increase in the expression of neuronal markers, and the number of neurons was found to have increased to the level observed in the Control group. Therefore, it was confirmed and verified that the transplantation of highly potent NSCs according to the present invention has an excellent effect in promoting the recovery and improvement of damaged neuronal cells.

### Example 15. In Vivo Ischemic Stroke Animal Model (Middle Cerebral Occlusion Rat Model, MCAO), Analysis of Reactive Gliosis Improvement by Injection of Stem Cells Selected According to the Present Invention (Astrocytes, Microglia)

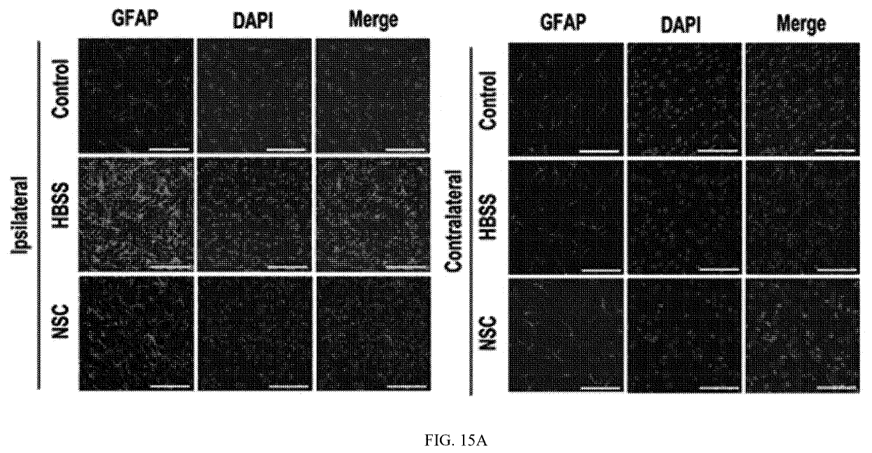

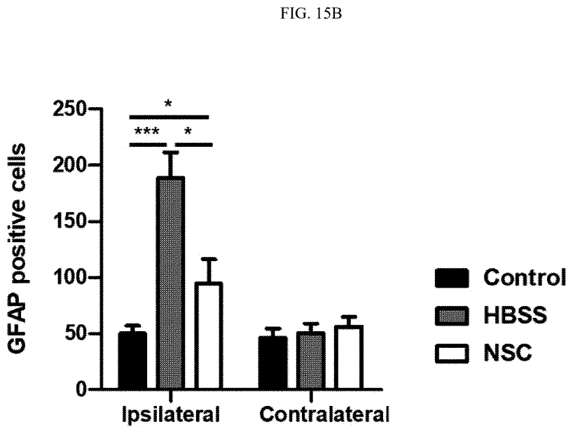

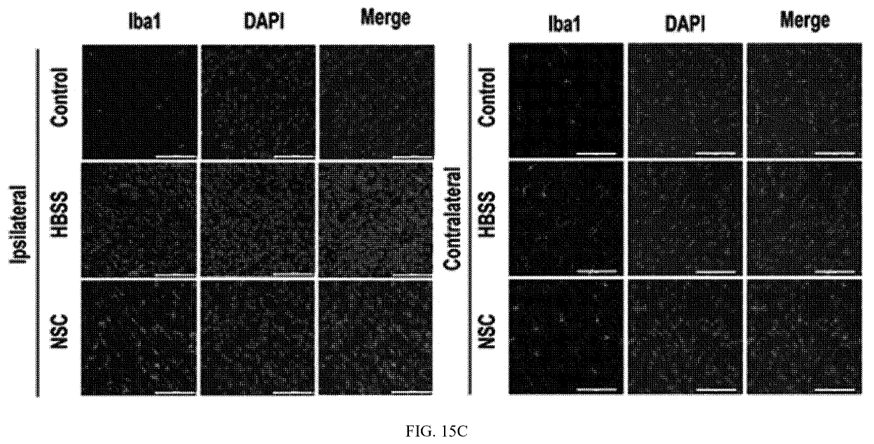

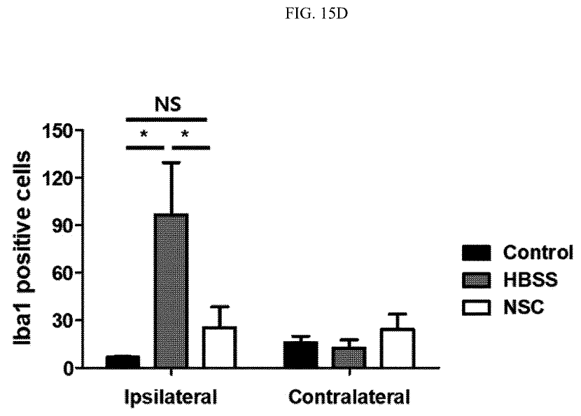

In the animal experiment conducted according to the protocol of Example 12-2, brain tissue was collected, and to evaluate the change in reactive gliosis induced by the injection of highly potent stem cells according to the present invention, immunohistochemical staining was performed on the damaged cerebral cortex area (see FIG. 14a) using GFAP antibody and Iba1 antibody, respectively. Through the immunohistochemical staining, the increase in reactive gliosis via astrocytes was evaluated by measuring the GFAP (glial fibrillary acidic protein) levels (FIG. 15a and FIG. 15b), and the increase in reactive gliosis via microglia was evaluated by measuring the Iba1 (ionized calcium-binding adapter molecule 1) levels (FIG. 15c and FIG. 15d). The immunofluorescence tissue staining was performed using the primary antibodies GFAP Ab ([NSJ Bioreagents (V2129)], dilution 1:100) or Iba1 Ab ([abcam (ab178846)], dilution 1:500), and was carried out in the same manner as described in Example 16, except for the use of these specific antibodies.

As shown in FIG. 15a, FIG. 15b, FIG. 15c, and FIG. 15d, the number of reactive astrocytes increased in the HBSS group compared to the Control group. However, in the group transplanted with highly potent neural stem cells according to the present invention, the expression of reactive astrocyte markers decreased compared to the HBSS group. Additionally, the number of reactive microglia increased in the HBSS group compared to the Control group. However, in the group transplanted with highly potent neural stem cells according to the present invention, the expression of reactive microglia markers decreased compared to the HBSS group. In other words, it was confirmed that the transplantation of highly potent neural stem cells selected according to the present invention resulted in significantly less reactive gliosis in the tissue, demonstrating a remarkable improvement in tissue damage.

### Example 16. Analysis of the Cell Death Improvement Effect by the Injection of Selected Neural Stem Cells According to the Present Invention in an In Vivo Ischemic Stroke Animal Model (Middle Cerebral Occlusion Rat Model, MCAO)

In the animal experiment conducted according to the protocol of Example 12-2, brain tissue was collected, and to evaluate the extent of cell death improvement in the damaged tissue by the injection of highly potent neural stem cells according to the present invention, the tissue was homogenized, and the expression of proteins related to cell survival and death, BAX, Bcl2, and cleaved caspase-3, was assessed via Western blotting.

The brain tissue was lysed in RIPA buffer (ChemCruz, sc-24948). The protein concentration of the samples was quantified using Bradford Protein Assay Dye (BIO-RAD, #5000006). Equal amounts of protein were loaded onto the OneGel PAGE Kit Plus (BIOSOLUTION) and separated according to the manufacturer's protocol using 1× Tris-Glycine-SDS buffer (DYNE BIO, CBT3141). The separated proteins were transferred onto a nitrocellulose membrane in 1× Tris-Glycine buffer (DYNE BIO, CBT3291) with methanol (DAEJUNG, 5558-4410). Protein separation and transfer were performed using the Tetra Vertical Electrophoresis and Blotting System (BIORAD). The membrane was blocked with 5% skim milk (CellNest, CNS109-0500) in Tris-buffered saline (DYNE BIO, CBT3051) containing Tween-20 (VWR LIFE SCIENCE, 0777-1L) at room temperature for 1 hour. The membrane was then incubated overnight at 4° C. with the primary antibody in 5% bovine serum albumin (BSA) (SIGMA, A-2153). The primary antibody information used is as follows: Bax Ab ([Santa Cruz (sc-7480)], dilution 1:1,000), Bcl-2 Ab ([Santa Cruz (sc-7382)], dilution 1:100), BAb ([Cell signaling (9664)], dilution 1:1,000), and Cleaved-caspase3 Ab ([Santa Cruz (sc-47778)], dilution 1:1,000). Then, the membrane was incubated at room temperature for 1 hour with horseradish peroxidase-conjugated goat anti-mouse or rabbit IgG secondary antibody (1:10,000). The detection kit solution (WESTSAVE up, LF-QC0101) was applied to the membrane, and then the signals were detected using X-ray film (AGFA) or a detection system (JPI Healthcare Co., JP-33). The bands were quantified using ImageJ software and normalized by the signal of β-actin.

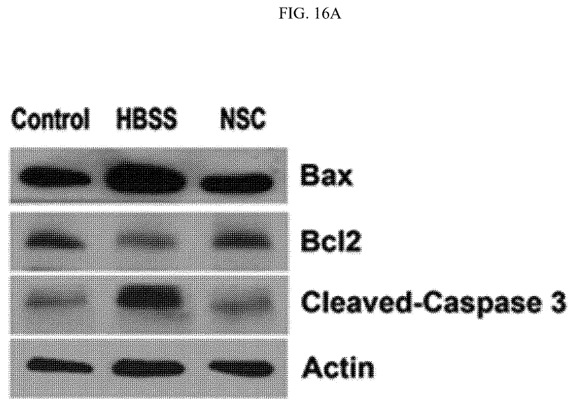

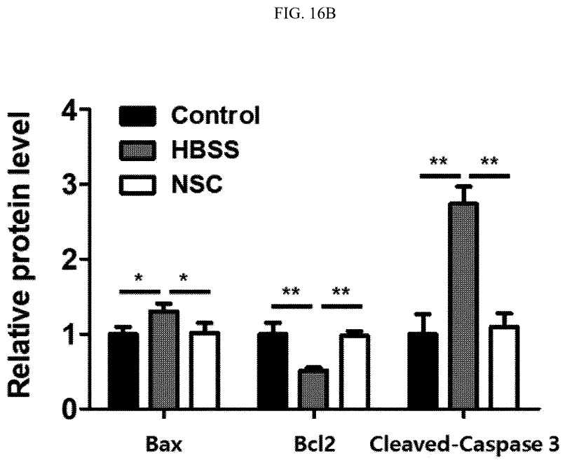

As shown in FIG. 16a and FIG. 16b, the Western blot results confirmed that, compared to the Control group, the expression of the cell death-related proteins Bax and Cleaved-caspase3 was increased in the HBSS group, while the expression of the survival-related protein Bcl2 was decreased. In contrast, compared to the HBSS group, the group transplanted with highly potent stem cells selected according to the present invention showed a decrease in the expression of Bax and Cleaved-caspase3, while the expression of Bcl2 increased. In particular, in the group transplanted with highly potent stem cells according to the present invention, the level of decrease in Bax and Cleaved-caspase3 expression, as well as the increase in Bcl2 expression, were found to be similar to the levels observed in the Control group.

Therefore, based on the above results, it was confirmed and verified that the injection of highly potent stem cells according to the present invention significantly reduces tissue damage caused by ischemia, demonstrating an excellent effect in alleviating tissue damage.

### Example 17. Analysis of Cerebral Vessel Recovery Effect Through Injection of Stem Cells Selected According to the Present Invention in an In Vivo Ischemic Stroke Animal Model (Middle Cerebral Occlusion Rat Model, MCAO)

In the animal experiment performed according to the protocol of Example 12-2, brain tissue was collected, and to evaluate the degree of cerebral vessel recovery through injection of highly potent stem cells according to the present invention, CD31 immunofluorescence staining (for vascular staining) was conducted in the cerebral cortex. Immunofluorescence staining d using CD31 Ab ([abcam (ab28364)], dilution 1:50) as the primary antibody, and was carried out in the same manner as in Example 14, except for this change.

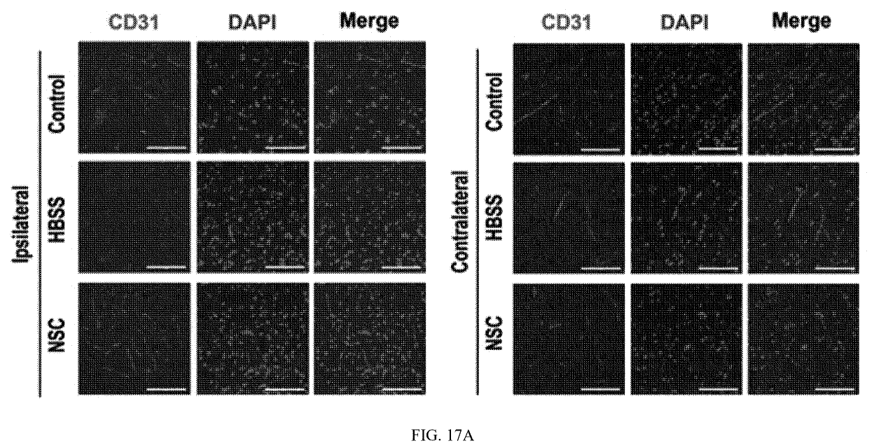

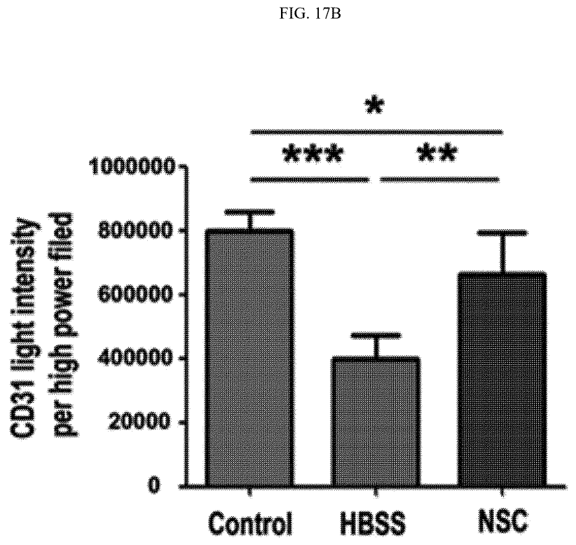

As shown in FIG. 17a and FIG. 17b, the CD31 immunohistochemical staining results revealed that, compared to the Control group, the vascular expression in the stroke-damaged tissue was significantly reduced in the HBSS group. However, in the group that received the highly potent stem cells selected according to the present invention, the vascular expression was significantly increased compared to the HBSS group. Based on these results, it was confirmed and verified that the transplantation of highly potent stem cells selected according to the present invention induces vascular formation, demonstrating a highly effective outcome. In contrast, in the tissue where cerebral infarction did not occur (contralateral tissue), there was no significant difference in the degree of vascular expression among the three groups.

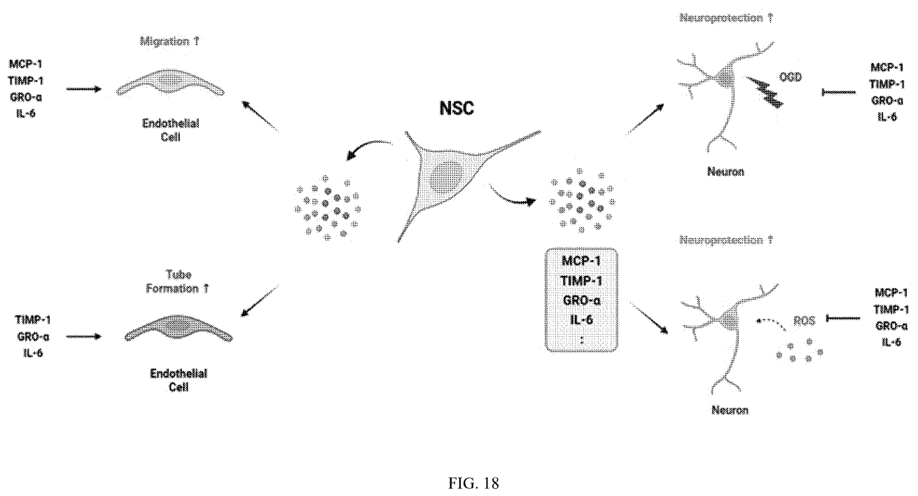

In summary (refer to FIG. 18), the inventors have confirmed that by selecting isolated stem cells through the efficacy factors of the present invention (MCP-1, TIMP-1, GROα, and IL-6), and transplanting the selected stem cells, they can effectively enhance the prevention of brain nerve damage and promote vascular regeneration in stroke, a typical neurological disease, as well as in brain damage that may occur secondary to it. Additionally, the present invention confirmed that not only the histological and biochemical recovery effects of the brain damaged by stroke, but also the behavioral sensory and motor abilities, were significantly improved. In other words, the specific neuroprotective ability of the factors in neuronal and endothelial cells, as well as their ability to promote vascular regeneration, were confirmed. Additionally, it was confirmed that the neural stem cells have a low potential to induce immune rejection and possess strong immunosuppressive abilities.

In other words, the inventors confirmed that the transplantation of neural stem cells selected according to the present invention demonstrates therapeutic effects for neurological disease, including spinal cord injury and stroke. This was verified through the increase in survival of neural and vascular cells in damaged tissues and the improvement of behavior through a reduction in reactive gliosis, immune cells, and cell death, which are caused by neuronal loss. Therefore, not only through the isolation and culture of neural stem cells, but also by identifying the specific efficacy factors secreted by the isolated and cultured neural stem cells, the inventors have proposed a new therapeutic strategy that enables more remarkable highly potent and high-performance treatment of neurological disease.

Taking all the above results into account (refer to FIG. 18), it is clearly understood by those skilled in the art that the specific combination of the four factors MCP-1, TIMP-1, GROα, and IL-6 in the present invention using neural stem cells significantly enhances the therapeutic efficacy of the neural stem cells in the treatment and symptom improvement of various neurological disease.

The description of the present invention provided above is for illustrative purposes, and those skilled in the art to which the present invention pertains will readily understand that the present invention can be modified into other specific forms without altering the technical spirit or essential features of the present invention. Therefore, the embodiments described above should be understood as being exemplary in all respects and not limiting in nature.

### INDUSTRIAL APPLICABILITY

According to the present invention, the application of stem cells exhibiting increased expression or activity of TIMP-1, MCP-1, GROα, and IL-6 factors or the use of substances that regulate (particularly upregulate) these factors in stem cells enables the treatment of neurological disease with high efficacy (potency) and efficiency. Thus, the present invention has industrial applicability.

1-52. (canceled)

53. A method for selecting highly potent stem cells for the treatment of neurological disease, wherein the method comprises the following steps:
(a) manufacturing a preparation for selecting highly potent stem cells for the treatment of neurological disease using a substance for measuring the expression levels of TIMP-1 (TIMP Metallopeptidase Inhibitor 1), MCP-1 (Monocyte Chemoattractant Protein-1), GROα (Growth-Regulated Oncogene alpha), and IL-6 (Interleukin 6) proteins or their genes; and
(b) measuring the expression levels of TIMP-1, MCP-1, GROα, and IL-6 proteins or their genes.

54. The method according to claim 53, wherein the TIMP-1, MCP-1, GROα, and IL-6 proteins or their genes are highly expressed in the highly potent stem cells for the treatment of neurological disease.

55. The method according to claim 53, wherein the stem cells are selected from the group consisting of mesenchymal stem cells, adult stem cells, dedifferentiated stem cells, embryonic stem cells, induced pluripotent stem cells, neural stem cells, tissue-derived stem cells, and any combination of two or more thereof.

56. The method according to claim 53, wherein the substance for measuring the expression levels of the proteins is an antibody specific to the proteins.

57. The method according to claim 53, wherein the expression levels of the genes are measured using a substance for measuring the mRNA expression levels of the genes.

58. The method according to claim 57, wherein the substance for measuring the mRNA expression levels is a probe or primer set that specifically binds to the mRNA.

59. The method according to claim 53, wherein the neurological disease is one or more selected from the group consisting of ischemic brain disease, degenerative neurological disease, nerve damage disease, peripheral nerve disorders, and traumatic brain injury.

60. The method according to claim 59, the method is characterized by one or more selected from the group consisting of:
wherein the ischemic brain disease is one or more selected from the group consisting of stroke, cerebral infarction, cerebral ischemia, cerebral embolism, cerebral thrombosis, transient ischemic attack, head trauma, brain circulatory metabolic disorders, brain coma, cerebral hemorrhage, neonatal intraventricular hemorrhage (IVH), cerebral arteriosclerosis, hypoxic brain injury, and neonatal hypoxic ischemic brain injury;
wherein the degenerative neurological disease is one or more selected from the group consisting of dementia, Alzheimer's disease, Parkinson's disease, progressive supranuclear palsy, multiple system atrophy, olivopontocerebellar atrophy (OPCA), Shy-Drager syndrome, striatonigral degeneration, Huntington's disease, amyotrophic lateral sclerosis (ALS), essential tremor, corticobasal ganglionic degeneration, diffuse Lewy body disease, Parkinson-ALS-dementia complex of Guam, and Pick's disease; and
wherein the nerve damage disease is one or more selected from the group consisting of epilepsy, spinal cord injury diseases, behavioral disorders, developmental disorders, intellectual disability, Down syndrome, and schizophrenia.

61. The method according to claim 53, wherein the method comprises the following steps:
(i) culturing stem cells;
(ii) manufacturing a preparation for selecting highly potent stem cells for the treatment of neurological disease using a substance for measuring the expression levels of TIMP-1 (TIMP Metallopeptidase Inhibitor 1), MCP-1 (Monocyte Chemoattractant Protein-1), GROα (Growth-Regulated Oncogene alpha), and IL-6 (Interleukin 6) proteins;
(iii) measuring concentrations of TIMP-1, MCP-1, GROα, and IL-6 proteins in the stem cell culture of step (i) using the preparation manufactured in step (ii); and
(iv) determining and classifying the stem cells as highly potent stem cells for neurological disease treatment if TIMP-1, MCP-1, GROα, and IL-6 proteins are highly expressed in the culture.

62. The method according to claim 61, wherein step (iv) comprises:
measuring the culture obtained in step (iii), and
if the TIMP-1 protein concentration is 6,000 μg/mL or higher, the MCP-1 protein concentration is 6,000 μg/mL or higher, the GROα protein concentration is 500 μg/mL or higher, and the IL-6 protein concentration is 50 μg/mL or higher, determining that the stem cells are highly potent stem cells.

63. A method for treating, or improving neurological disease; or for inducing angiogenesis, wherein the method comprises administering to a subject in need thereof a pharmaceutically effective amount of composition comprising, as the active ingredient:
stem cells overexpressing TIMP-1, MCP-1, GROα, and IL-6 genes or their proteins, or a culture thereof; or
isolated stem cells, and a substance that increases the expression or activity of TIMP-1, MCP-1, GROα, and IL-6 genes or their proteins.

64. The method according to claim 63, wherein the culture is a conditioned medium comprising TIMP-1, MCP-1, GROα, and IL-6 proteins secreted by the stem cells overexpressing TIMP-1, MCP-1, GROα, and IL-6 genes or their proteins.

65. The method according to claim 63, wherein the stem cells overexpressing TIMP-1, MCP-1, GROα, and IL-6 genes or their proteins are selected from the group consisting of mesenchymal stem cells, adult stem cells, dedifferentiated stem cells, embryonic stem cells, induced pluripotent stem cells, neural stem cells, tissue-derived stem cells, and any combination of two or more thereof.

66. The method according to claim 63, wherein the substance that increases the expression or activity of the proteins is one or more expression vectors comprising a polynucleotide encoding the TIMP-1 protein; a polynucleotide encoding the MCP-1 protein;
a polynucleotide encoding the GROα protein; and a polynucleotide encoding the IL-6 protein.

67. The method according to claim 63, wherein the composition is for the treatment of cerebrovascular disease.

68. The method according to claim 67, wherein the cerebrovascular disease is one or more selected from the group consisting of cerebral infarction, cerebral ischemia, stroke, vascular dementia, cerebral hemorrhage, subarachnoid hemorrhage, and leukodystrophy.

69. A method for promoting the activity of stem cells for the treatment of neurological disease, comprising a step of treating a substance that increases the expression or activity of TIMP-1, MCP-1, GROα, and IL-6 genes or their proteins.

70. The method according to claim 69, wherein the substance that increases the expression or activity of the proteins is one or more expression vectors comprising a polynucleotide encoding the TIMP-1 protein; a polynucleotide encoding the MCP-1 protein;
a polynucleotide encoding the GROα protein; and a polynucleotide encoding the IL-6 protein.

71. The method according to claim 69, wherein the activity of the stem cells is one or more selected from the group consisting of self-renewal, cell protection ability, inhibition of cell apoptosis, vascular endothelial cell migration, angiogenesis, anti-inflammatory activity, and inhibition of reactive gliosis.

72. The method according to claim 69, wherein the step of treating is a step of treating isolated stem cells with a substance that increases the expression or activity of TIMP-1, MCP-1, GROα, and IL-6 genes or their proteins.


SEQUENCE LISTING (8 sequences)

SEQ ID NO: 1 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 2 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 3 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 4 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 5 - DNA, 769 bp, Homo sapiens
  source 1..769: mol_type = genomic DNA
gccatcgccgcagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaagctgaagcctgcacagtgtccaccctgttcccactcccatctttcttccggacaatgaaataaagagttaccacccagca

SEQ ID NO: 6 - DNA, 760 bp, Homo sapiens
  source 1..760: mol_type = genomic DNA
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 7 - DNA, 1174 bp, Homo sapiens
  source 1..1174: mol_type = genomic DNA
acagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatagaaaaaactcgtttgatttttggggggaaacaagggctacctttactggaaaatctggtgatttatatcaata

SEQ ID NO: 8 - DNA, 1127 bp, Homo sapiens
  source 1..1127: mol_type = genomic DNA
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca